(19) United States
(12) Patent Application Publication
(10) Pub. No.: US 2025/0222469 A1
(43) Pub. Date: Jul. 10, 2025

(54) CENTRIFUGE AND MIXING SYSTEM FOR A CELL PROCESSING APPARATUS

(71) Applicant: Trenchant BioSystems, Inc., Stockton, CA (US)

(72) Inventors: Philip H. Coelho, Sacramento, CA (US); Paul Getchel, Fallon, NV (US); Jonathan Ellis, Stockton, CA (US); Benjamin T. Edmundson, Orangevale, CA (US); Samuel G. Miller, Folsom, CA (US); Duncan MacGregor, Shingle Springs, CA (US); Michael S. Wilk, Rocklin, CA (US); Joël Bodenmann, Seewjinenstrasse (CH)

(21) Appl. No.: 19/004,239

(22) Filed: Dec. 28, 2024

Related U.S. Application Data
(60) Provisional application No. 63/616,710, filed on Dec. 31, 2023.

(51) Int. Cl. B04B 5/04; B04B 13/00; B04B 15/02

(52) U.S. Cl. CPC B04B 5/0407; B04B 13/00; B04B 15/02; B04B 2013/006

(57) ABSTRACT

A multi-functional centrifuge bucket designed for use with a cell processing cassette (CPC) in automated cell processing platforms. The centrifuge bucket features a secure mounting interface for precise CPC alignment, an integrated temperature control system to maintain stable conditions, and a pivoting mechanism for tilting or rotation to enable uniform mixing. Real-time monitoring of cell sedimentation and distribution is facilitated by optical sensors, while a data communication system supports automated adjustments to centrifugation and mixing parameters. The heating mechanism includes thermal insulation layers and advanced fail-safe controls, ensuring precise temperature regulation within the CPC while preventing external overheating. Methods of use include spinoculation, sedimentation, and precise fluid mixing, enabled by controlled motion and feedback systems. This platform ensures efficient, contamination-free processing of cell solutions, making it ideal for gene therapy, advanced cell selection, and therapeutic cell production.

### RELATED APPLICATIONS

This application claims priority from the United States Provisional Application with Ser. No. 63/616,710 which was filed on Dec. 31, 2023. The Provisional Application is hereby incorporated by reference in its entirety.

### TECHNICAL FIELD OF THE DISCLOSURE

This disclosure is generally related to cell therapies, and more particularly a device capable of separating target cells from non-target cells in blood or blood products and genetically modifying the target cells for clinical use in the field of cell and gene therapy.

### BACKGROUND

Gene-engineered autologous cell therapies, which utilize a patient's own cells re-engineered and expanded ex vivo, represent a revolutionary approach to addressing complex diseases with high mortality and morbidity rates. These therapies leverage genetic modifications to correct underlying defects, offering transformative health outcomes for conditions where conventional treatments are limited or ineffective. Such gene-modified cell therapies, including those targeting hematopoietic cells such as T-cells, NK (Natural Killer) cells, and stem cells, have become a cornerstone of modern precision medicine, enabling targeted and durable interventions.

The field has witnessed rapid advancements, propelled by recent regulatory approvals and a robust development pipeline. Since 2017, multiple CAR-T cell therapies have received FDA approval for hematologic cancers, with over 1000 additional gene-modified therapies in U.S. clinical trials by late 2019. Beyond CAR-T therapies, CD34+ hematopoietic stem cells (HSCs) have shown similar promise for inherited disorders, including beta-thalassemia and severe combined immunodeficiency (SCID), with two therapies approved in the EU and over 31 pediatric trials underway in the U.S. as of 2019. Market forecasts predict a substantial expansion, with the global gene therapy market projected to grow from $18 billion in 2023 to over $97 billion by 2033. This growth reflects the increasing potential and demand for gene-modified cell therapies.

Originally developed to combat blood cancers, CAR-T therapies are now being explored for a broader spectrum of conditions, significantly expanding the pool of potential patients. These therapies have demonstrated notable promise in addressing a wide range of indications:

Gene-modified immune cells, particularly CAR-T cells and NK cells, provide precise targeting and elimination of cancer cells in blood and solid tumor cancers, offering a superior alternative to traditional treatments such as chemotherapy, radiation stem cell transplants for hematopoietic reconstitution, which are often associated with severe side effects and limited efficacy.

Available hemoglobinopathies include the use of gene-modified hematopoietic stem cells that present potential curative treatments for genetic conditions such as sickle cell disease and beta-thalassemia by addressing the fundamental defects in hemoglobin production. There are also treatments available for autoimmune diseases through the engineering of immune cells to regulate or eliminate malfunctioning immune responses, gene-modified therapies offer new hope for chronic autoimmune conditions like lupus, rheumatoid arthritis, and multiple sclerosis, which currently lack definitive cures.

There is emerging research showing the role of senescent cells in aging and associated diseases. Gene-modified cell therapies can selectively target and clear these cells, mitigating age-related conditions, improving quality of life, and potentially extending healthy lifespan.

These advancements signify a paradigm shift in medicine, emphasizing the precision and adaptability of gene modification to address the root causes of complex diseases rather than merely managing symptoms. However, the potential of gene-modified cell therapies is hindered by significant challenges in manufacturing, scalability, and accessibility.

Challenges in Current Manufacturing Processes—Despite their clinical promise, the production of gene-modified cell therapies is constrained by inefficiencies, high costs, and limited scalability. The reliance on outdated technologies and labor-intensive workflows exacerbates these issues, making it difficult to meet the growing demand for these therapies.

Obsolete and Complex Technologies—Conventional manufacturing relies on multiple different antiquated equipment and processes that are inefficient, prone to cell loss, and at risk of contamination that are reported to result in up to 92.1% T-cell loss, requiring additional ex-vivo expansion steps to recover cell populations. These steps increase production time, variability, cell exhaustion, and contamination risks. Traditional bead-based systems, which permanently attach magnetic beads to cells, are inefficient and lack the flexibility required for sequential selections, limiting their utility for producing specialized cell subsets like memory T-cells. Similarly, flow sorters, while capable of achieving high purity through multi-parametric sorting, are unsuitable for clinical-scale production due to their low throughput.

Lengthy and Inefficient Processing—The current “vein-to-vein” timeline for CAR-T production is 30-40 days, much of which is spent on ex-vivo expansion to compensate for cell losses. These extended timelines increase the risk of cell exhaustion and depletion of critical subpopulations, such as memory T-cells, which are essential for sustained therapeutic efficacy. Centralized manufacturing further exacerbates delays, as cells must be frozen and then transported between facilities for different processing stages, reducing cell viability, causing manufacturing challenges due to tumor cell lysis, and increasing logistical complexity.

High Costs and Limited Accessibility—The inefficiencies of traditional methods translate into prohibitively high costs, with therapies often priced between $350,000 and $500,000 per patient, and some gene therapies exceeding $4 million per dose. These costs, coupled with the slow production process, restrict access to these treatments for many patients and limit their adoption within healthcare systems.

Limited Capacity for Clinical and Research Applications—Existing production systems are not equipped to handle the demands of large-scale clinical applications or rapid iteration for research purposes. This limited capacity slows the development and testing of next-generation therapies, hindering innovation and the ability to serve broader patient populations.

Worsening Over Time—As gene-modified cell therapies are adapted to treat more diverse conditions—such as solid tumors, autoimmune diseases, neuromuscular disorders, and age-related conditions—the shortfalls of current manufacturing approaches will become increasingly pronounced. This growing demand highlights the urgency for scalable, efficient, and cost-effective production solutions.

Emerging Solutions and Remaining Challenges—Innovative approaches are being developed to address these challenges. Alternatives such as automation, non-viral vectors, and stable cell line platforms for recombinant adeno-associated virus (rAAV) production show promise in enhancing scalability, consistency, and cost-efficiency. However, these methods are often in the early stages and face hurdles in regulatory approval and commercial scalability. For instance, while stable rAAV producer cell lines can streamline production timelines and improve reliability, they must overcome difficulties in balancing cell viability with productivity and stability, particularly with toxic rep/helper genes.

### The Present Invention

The present invention seeks to address the critical challenges faced by current gene-modified cell therapy manufacturing processes, offering a streamlined, reliable, and economically viable platform to produce these transformative therapies. The present invention introduces a transformative platform designed to overcome the limitations of conventional manufacturing processes. Sometimes referred to herein as an AutoCell Platform (ACP), the platform integrates advanced automation, closed-loop processing, and innovative technologies to streamline workflows, reduce cell loss, and compress production timelines, reducing costs by an order of magnitude. The key objectives of the invention are as follows:

It is a first objective of the invention to integrate advanced automation with a functionally closed-system technology, enabling a highly efficient and controlled production process. By minimizing human intervention, this closed-loop design significantly reduces contamination risks and manual errors, enhancing the safety, consistency, and scalability of gene-modified cell therapy manufacturing. Traditional methods, which often require 30-40 days to complete a batch due to labor-intensive steps like washing, selection, activation, transduction, and formulation, are compressed to less than 3 days using this platform. The elimination of the requirement for ex-vivo expansion to replace cells lost in manufacturing, improves efficiency, and lowers manufacturing costs to approximately one-tenth of conventional levels, making these therapies more affordable and accessible.

Another objective of the invention is to provide a platform that introduces an automated spinoculation process, a novel centrifuge-based method that combines spinning and inoculation (i.e., Spinoculation) to significantly improve genetic material uptake by target cells. Conventional methods typically achieve only 25-35% efficiency. The invention's automated spinoculation consistently achieves 70% efficiency, improving the quality and reliability of gene-modified cells. This technique is particularly critical for the gene modification of target cells within a single vessel, ensuring that high levels of genetic material are successfully delivered into the target cells with minimal variability.

Another objective of the invention is to provide a scalable platform having a modular, standardized design that allows for standardized manufacturing across a wide variety of clinical and research settings. Unlike traditional bespoke systems, which are tailored to specific facilities and therapies, the platform can be replicated and deployed in diverse locations, including smaller or remote healthcare centers. This adaptability ensures that patients can receive treatment directly at their treatment center, reducing logistical challenges like transportation, freezing, and storage that often compromise cell viability. By enabling both centralized and decentralized manufacturing models, the invention expands the availability of advanced therapies while maintaining high levels of consistency and quality.

Another objective of the invention is to provide precision in cell selection and modification, such as microbubble-assisted cell selection (MBCSA) and aptamer-guided targeting, to achieve unparalleled precision in the isolation and activation of specific cell populations. These advanced methods enable the production of high-quality CAR-T cells, NK (Natural Killer) cells, and gene-modified stem cells by focusing on key subpopulations that maximize therapeutic efficacy. Unlike conventional techniques that often result in high levels of cell loss and contamination, the ACP's precise selection and modification processes ensure optimal purity and functionality of the final therapeutic product.

Microbubble-assisted cell selection (MBCSA) is a critical innovation within the ACP, allowing for sequential and highly specific isolation of target cells. By utilizing microbubbles that can attach and provide buoyancy to target cells, the platform separates selected cells from the bulk population, enabling multi-step enrichment without compromising cell viability. Then after separation has occurred, and non-target cells removed, the buoyancy can be removed by imploding the microbubbles with modest pressure increase in the container to then allow the target cells to undergo further processing steps. Similarly, aptamer-guided targeting employs molecular binding agents with high specificity for particular cell surface markers, ensuring precise activation and retention of the desired cell subsets.

The prevision of the ACP is particularly significant for the development of CAR-T cell therapies, where the composition and quality of T-cell subsets directly influence clinical outcomes. CAR-T products derived from carefully enriched subsets, such as CD8+ cytotoxic T-cells and CD4+ helper T-cells, are known to exhibit superior antitumor effects. The ACP has demonstrated its ability to achieve successful enrichment of CD8+ T-cells from a general T-cell population, highlighting its capacity to enhance the therapeutic potency of gene-modified cell products.

Another objective of the invention is to provide a versatile and customizable platform that may accommodate a variety of cell types and therapies, including hematopoietic stem cells, T-cells, and NK cells. Customizable protocols enable the system to adapt to the unique requirements of different therapies, from CAR-T treatments for cancer to regenerative therapies for age-related conditions. This versatility makes the platform a valuable tool across a spectrum of gene-modified therapies.

Another objective of the invention is to provide a compact and automated platform, enabling its deployment in FDA-licensed transplant centers or other localized healthcare facilities. This proximity to patients reduces patient accessibility and logistical delays, minimizes cell viability loss due to freezing and thawing, and accelerates treatment availability. By facilitating point-of-care (POC) manufacturing, the platform enhances access to advanced therapies for underserved populations and eliminates many of the inefficiencies inherent in centralized production models.

Another objective of the invention is to provide research and clinical support. The platform's automated and standardized processes free researchers to focus on developing effective gene constructs rather than labor-intensive manufacturing tasks. This simplification accelerates translational research, allowing scientists to rapidly iterate on therapeutic vector designs and bring innovative therapies to clinical trials faster. By supporting the 592 cell biology research facilities in the United States, the invention fosters a more dynamic and productive research environment.

Another objective of the invention is to provide a platform incorporating robust and automated quality control measures, including automated pressure decay testing for filter integrity checks, and septa disinfection modules for aseptic transfer operations, ensuring compliance with stringent FDA standards. These built-in features reduce the risk of contamination, batch failure, and regulatory delays. The platform's integrated data tracking and lot release records collected real-time and provided concurrent to harvest of gene-modified target cells, further streamline regulatory filings, expediting approvals for new therapies and ensuring that each batch meets rigorous quality standards.

It is yet another objective of the invention to address the high costs, inefficiencies, and logistical challenges that have historically limited the adoption of gene-modified therapies. By reducing production costs and compressing timelines, the platform significantly enhances the scalability of these therapies, making them viable options for a broader patient population. This transformation expands market opportunities and positions the platform as a foundational technology for next-generation cell therapies.

It is yet another objective of the invention to provide innovative capabilities to support the development of therapies for diseases previously considered untreatable, including solid tumors, autoimmune conditions, and age-related degenerative disorders. Faster production timelines enable patients with aggressive diseases to receive timely treatments, including at time of initial diagnosis, while reduced costs make these therapies a sustainable option for chronic conditions. The platform's flexibility and precision unlock new therapeutic possibilities, opening the door to lifesaving and life-extending treatments.

It is a still further objective of the invention is to provide a platform supporting both centralized and decentralized manufacturing approaches, providing flexibility to meet diverse clinical and logistical needs. Centralized facilities benefit from streamlined, high-throughput production, while decentralized setups enable faster vein-to-vein times, reducing the overall timeline from apheresis to reinfusion and improving patient outcomes. This dual capability transforms the logistics of cell therapy manufacturing, ensuring that therapies can be delivered where and when they are needed.

The present invention represents a transformational approach to the production of gene-modified cell therapies. By addressing the inefficiencies, high costs, and scalability challenges of traditional methods, the platform improves access to these life-saving treatments for a broader range of patients. Its innovative design enhances research, accelerates regulatory approvals, and enables the development of new therapeutic options, ultimately advancing the frontiers of precision medicine and expanding the global impact of gene-modified therapies.

### SUMMARY OF THE INVENTION

The AutoCell Platform (ACP) is a fully automated, functionally closed cell processing system engineered to facilitate aseptic, FDA-compliant transfers of reagents, buffers, culture media, and gases through various processing cassettes. The platform employs advanced robotic controls to coordinate precise movements and interactions among the cassettes. This integration of robotic precision with a sterile, functionally closed environment makes the ACP highly effective for manufacturing CAR T-cells and other cell-based therapies. By minimizing contamination risks and optimizing automation, the ACP ensures the production of high-quality cell products suitable for clinical applications.

The system employs a series of cassettes including among others a Cell Processing Cassette (CPC) in which target cells are introduced, selected, enriched, genetically modified, washed and concentrated.

A robotic control system ensures that each cassette operates in a coordinated sequence, maintaining aseptic conditions and eliminating the need for manual handling. This system minimizes contamination risk while enhancing precision and efficiency in reagent and fluid management. In one embodiment, the ACP's robotic mechanism precisely controls the positioning and operation of the supporting cassettes:

Transfer Syringe Cassette (TSC): This cassette manages the precise transfer of reagents and fluids into the CPC, enabling accurate volumetric dosing for processes such as washing, activation, and transduction.

Reagent/Sample Cassette (RSC): The RSC houses a plurality of pre-filled reagent vials, preferably six and preferably with a volume capacity of up to 20 mL. In some embodiments, these vials contain essential components such as microbubbles, vectors, and linkers. Additionally, the RSC contains designated sample vials, ready to receive cell samples for quality control (QC) analysis throughout the processing cycle.

Process Fluids Cassette (PFC): The PFC stores various process fluids, such as buffers and culture media, required at different stages of cell processing. These fluids are automatically transferred to the CPC as needed, supporting cell washing, sedimentation, and formulation.

The ACP preserves more cells than traditional centralized manufacturing, where cells often experience losses due to freezing, transport, and multiple transfers. A central air pressure system enables precise fluid movement and sterile transfer, further ensuring that cells remain uncontaminated.

Upon completion of manufacturing, the ACP automatically compiles and publishes a QC batch record that includes all tracked parameters and relevant data for that batch. This streamlined record enables rapid access and review, ensuring compliance with regulatory requirements while minimizing the time required for documentation. Pharmaceutical cell therapy facilities, particularly those specializing in CAR T-cell production, often employ more QC staff than manufacturing staff due to the traditional approach of exhaustive record review. The ACP's integrated QC automation and release by exception capabilities address this imbalance, allowing facilities to allocate resources more efficiently and reduce costs associated with cell therapy manufacturing.

### BRIEF DESCRIPTION OF THE FIGURES

In order to enhance the clarity and improve the understanding of the various elements and embodiments shown herein, the figures have not necessarily been drawn to scale. Furthermore, elements that are commonly known and well understood to those in the industry are not depicted in order to provide a clear view of the various embodiments of the invention; thus, the drawings are generalized in form for the purpose of clarity and conciseness.

FIG. 1 shows a perspective view of a cell processing device in a closed mode in accordance with the preferred embodiment of the present invention;

FIG. 2 shows perspective views of the cell processing device in an open mode;

FIG. 3 shows a cross-sectional side view of the cell processing device depicted in FIGS. 1 and 2 illustrating a centrifuge bucket in accordance with the preferred embodiment of the present invention;

FIG. 4 shows a perspective view of multiple cassettes relative to a lower housing assembly of the cell processing device depicted in FIGS. 1-3 in accordance with the preferred embodiment of the present invention;

FIG. 5 shows a perspective view of a flask and multiple cassettes wherein the depiction of the upper housing assembly and the lower housing assembly is removed in accordance with the preferred embodiment of the present invention;

FIGS. 6 and 7 show two operating modes of a centrifuge of the cell processing device in accordance with the preferred embodiment of the present invention;

FIG. 8 depicts a perspective view of the top surface of the CPC according to a preferred embodiment;

FIG. 9 is a perspective view of the top surface of the CPC according to an alternative embodiment;

FIGS. 10A and 10B are an exploded perspective view of three filters according to an embodiment of the invention;

FIG. 11 shows a partial exploded view of an alternative embodiment of a centrifuge bucket;

FIG. 12 shows an exploded view of a harvest valve assembly;

FIGS. 13-15 show exploded views of the CPC according to an embodiment of the invention;

FIGS. 16 and 17 are cross-sectional views of the CPC;

FIG. 18 is a cross-sectional side view of the CPC according to an alternative embodiment of the invention;

FIG. 19 shows a plan view of the CPC according to an embodiment of the invention;

FIG. 20 shows a side view of the CPC according to an embodiment of the invention and with section lines D-D, E-E and F-F shown in more detail in FIGS. 26, 27 and 28, respectively;

FIGS. 21-25 show cross-sectional views of CPC according to embodiments of the invention;

FIG. 26 shows the top view of the filters of the CPC according to an embodiment of the invention shown in cutline D-D in FIG. 20;

FIG. 27 depicts a top view of the main chamber of the CPC according to an embodiment of the invention shown in cutline E-E in FIG. 20;

FIG. 28 depicts a top view of the rotation valve of the CPC according to an embodiment of the invention shown in cutline F-F in FIG. 20;

FIG. 29 depicts a cross-sectional view of the rotation valve of the CPC showing a section of recovery tubing;

FIG. 30 is a top perspective view of the CPC;

FIG. 31 is a top cross-sectional view of the CPC showing section lines A-A, B-B and C-C, which define the views provided in FIGS. 32, 33 and 34;

FIG. 32 is a side view of the CPC showing section lines D1-D1, E1-E1 and F1-F1;

FIG. 33 is a cross-sectional view from FIG. 31 section line B-B;

FIG. 34 is a cross-sectional view from FIG. 21 section line C-C;

FIG. 35 is a cross-sectional view from FIG. 32 section line D1-D1;

FIG. 36 is a cross-sectional view from FIG. 32 section line E1-E1;

FIG. 37 is a cross-sectional view from FIG. 32 section line F1-F1;

FIG. 38 shows a plan view of the CPC according to an embodiment of the invention;

FIG. 39 shows is a cross-sectional of the CPC;

FIG. 40A shows a perspective view of a process fluids cassette (PFC) in accordance with the preferred embodiment of the present invention;

FIG. 40B shows a plan view of the PFC;

FIG. 40C shows a first cross section view of the PFC, and FIG. 41 shows a cross-sectional side view of the PFC in accordance with section line F-F from FIG. 40B;

FIG. 41 shows a cross-sectional side view of PFC 204 in accordance with section line F-F from FIG. 40B.

FIG. 42A shows a perspective view of a Reagent/Sample Cassette (RSC) in accordance with the preferred embodiment of the present invention;

FIG. 42B shows a plan view of the Reagent/Sample Cassette (RSC);

FIGS. 43A-43C show various views of a Transfer Syringe Cassette (TSC) in accordance with the preferred embodiment of the present invention;

FIGS. 44A-44B show cross-sectional side views of reagent syringes of the TSC interacting with the vial cassette in accordance with the preferred embodiment of the present invention;

FIGS. 44C-44D show cross-sectional side views of sampling syringes of the TSC interacting with the processing and vial cassettes in accordance with the preferred embodiment of the present invention;

FIG. 45A shows an actuator including a first gripping mechanism and a second gripping mechanism in accordance with the preferred embodiment of the present invention;

FIG. 45B shows the second gripping mechanism of the actuator that moves downward from the position illustrated in FIG. 45A in accordance with the preferred embodiment of the present invention;

FIG. 45C shows the first gripping mechanism of the actuator having moved downward and fingers of the first gripping mechanism having moved to a closed state in accordance with the preferred embodiment of the present invention;

FIG. 45D shows the movement of the actuator towards downward to push a needle of the reagent syringe out of a bottom end of the TSC in accordance with the preferred embodiment of the present invention;

FIG. 45E shows the movement of the second gripping mechanism towards upward direction while the first gripping mechanism remains stationary in accordance with the preferred embodiment of the present invention;

FIG. 45F shows the movement of the actuator towards upward direction to draw the needle in accordance with the preferred embodiment of the present invention;

FIG. 45G shows a configuration of the actuator to push the reagent syringe back downward to engage the needle into another cassette in accordance with the preferred embodiment of the present invention;

FIG. 45H shows the second gripping mechanism that being moved downward to press a plunger downward to expel fluids into other cassette in accordance with the preferred embodiment of the present invention;

FIG. 45I shows the actuator after moving upward to retract the needle into the TSC in accordance with the preferred embodiment of the present invention;

FIGS. 45J-45K show the actuator that being disengaging the first and the second gripping mechanisms and from the reagent syringe in accordance with the preferred embodiment of the present invention;

FIG. 46 shows a cross-sectional side view illustrating how the Process Fluids Cassette (PFC) transfers fluid to the CPC in accordance with the preferred embodiment of the invention;

FIGS. 47-48 are a perspective view of exemplary CAN-bus cabling according to an alternative embodiment of the invention;

FIGS. 49A-49B are cross sectional views of a centrifuge wherein the centrifuge bucket is in its mixing mode in accordance with the preferred embodiment of the present invention;

FIG. 49C is a cross-sectional view of a centrifuge when in rotation in accordance with the preferred embodiment of the present invention;

FIG. 49D is a cross-sectional view of a centrifuge when stationary in accordance with the preferred embodiment of the present invention;

FIG. 50 shows features of the heating mechanism of the centrifuge bucket in accordance with the preferred embodiment of the present invention;

FIGS. 51A and 51B show utilization of microbeads to facilitate a unique compression and release cycle for cell selection and concentration in accordance with the preferred embodiment of the present invention;

FIG. 52 shows an optical determination of valve rotation utilizing an optical encoder in accordance with the preferred embodiment of the present invention;

FIGS. 53A-53B show four primary rotational positions for CPC valves in accordance with the preferred embodiment of the present invention;

FIG. 54 shows the cell processing cassette in an active mode in accordance with the preferred embodiment of the present invention;

FIGS. 55A-55J show the CPC valve rotating through a series of positions to facilitate the step-by-step processing involved in CAR T-cell production in accordance with the preferred embodiment of the present invention;

FIG. 56 shows plan view of a portion of the CPC with components highlighted as part of the aseptic transfer process;

FIG. 57A is a perspective view of a portion of the ACP with a waste container;

FIG. 57B shows a carbon dioxide enriched container used as part of the aseptic transfer process;

FIGS. 58A-58D show the cell suspension when the CPC is being tilted in accordance with the preferred embodiment of the present invention;

FIG. 59A a shows cutline H-H through the CPC in accordance with the preferred embodiment of the present invention;

FIG. 59B shows a cross section along H-H illustrating a cell suspension fluid in the main chamber in accordance with the preferred embodiment of the present invention;

FIG. 59C shows movement of fluid toother chamber in accordance with the preferred embodiment of the present invention;

FIG. 59D shows the fluid from the main chamber that continues to urge the fluid upwards in the standpipe in accordance with the preferred embodiment of the present invention;

FIG. 60A shows a radar system in use with the CPC in accordance with the preferred embodiment of the present invention

FIG. 60B shows a S shaped channel that directs radar from a radar emitter positioned above and centered on the CPC cover in accordance with the preferred embodiment of the present invention;

FIG. 60C shows a rod for use with the radar system shown in FIGS. 60A and 60B in accordance with the preferred embodiment of the present invention; and

FIGS. 61A and 61B show a perspective view of a CPC with a sealed stub of tubing coiled in FIG. 61A and in uncoiled form in FIG. 61B.

### DETAILED DESCRIPTION OF THE DRAWINGS

In the following discussion that addresses a number of embodiments and applications of the present invention, reference is made to the accompanying drawings that form a part hereof, and in which is shown by way of illustration specific embodiments in which the invention may be practiced. It is to be understood that other embodiments may be utilized, and changes may be made without departing from the scope of the present invention.

Various inventive features are described below that can each be used independently of one another or in combination with other features. However, any single inventive feature may not address all or any of the problems discussed above. Further, one or more of the problems discussed above may not be fully addressed by any of the features described below.

As used herein, the singular forms “a”, “an” and “the” include plural referents unless the context clearly dictates otherwise. “And” as used herein is interchangeably used with “or” unless expressly stated otherwise. As used herein, the term “about” means+/−5% of the recited parameter. All embodiments of any aspect of the invention can be used in combination, unless the context clearly dictates otherwise.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “wherein”, “whereas” “above,”, “below”, and words of similar import, when used in this application, shall refer to this application as a whole and not to any particular portions of the application.

The description of embodiments of the disclosure is not intended to be exhaustive or to limit the disclosure to the precise form disclosed. While the specific embodiments of, and examples for, the disclosure are described herein for illustrative purposes, various equivalent modifications are possible within the scope of the disclosure, as those skilled in the relevant art will recognize.

This application describes an AutoCell Platform (ACP), an advanced, fully automated, and functionally closed cell processing system specifically designed to perform aseptic and FDA-compliant transfers of critical reagents, buffers, culture media, and gases through the lid of the CPC. Leveraging robotic controls, the platform orchestrates precise movements and coordinated interactions between its integral components, including the Transfer Syringe Cassette (TSC), Reagent/Sample Cassette (RSC), and Process Fluids Cassette (PFC). By seamlessly integrating robotic precision with a sterile, functionally closed processing environment, the ACP ensures high-quality, contamination-free production of CAR T-cells and other cell-based therapies. This innovative system not only mitigates contamination risks but also streamlines the manufacturing workflow, enabling efficient and consistent production of therapeutic-grade cell products for clinical applications.

The ACP works as a functionally closed system. A functionally closed system refers to a controlled environment that operates without exposure to external contamination. All material transfers into or out of the system are performed aseptically or through sealed mechanisms designed to maintain the sterile integrity of the internal processes. Sterile Maintenance of the CPC Interior through the use of aseptic connectors, sterile transfer ports, and sealed pathways ensures sterility during all operations. The internal environment is also completely isolated from external contamination risks. Finally, the system is designed to support material exchange without compromising sterility, ensuring consistent and reliable functionality.

The several embodiments of the invention described herein solve at least the technical problem of applying multiple cell processing steps autonomously without the need for manual intervention by lab personnel. In particular, a cell processing device is described that may be configured to separate target cells from non-target cells and then apply changes to the separated target cells. The ACP may be configured to operate with many different types of target cells including at least but not limited to T cells, NK cells and CD34+ HPSCs.

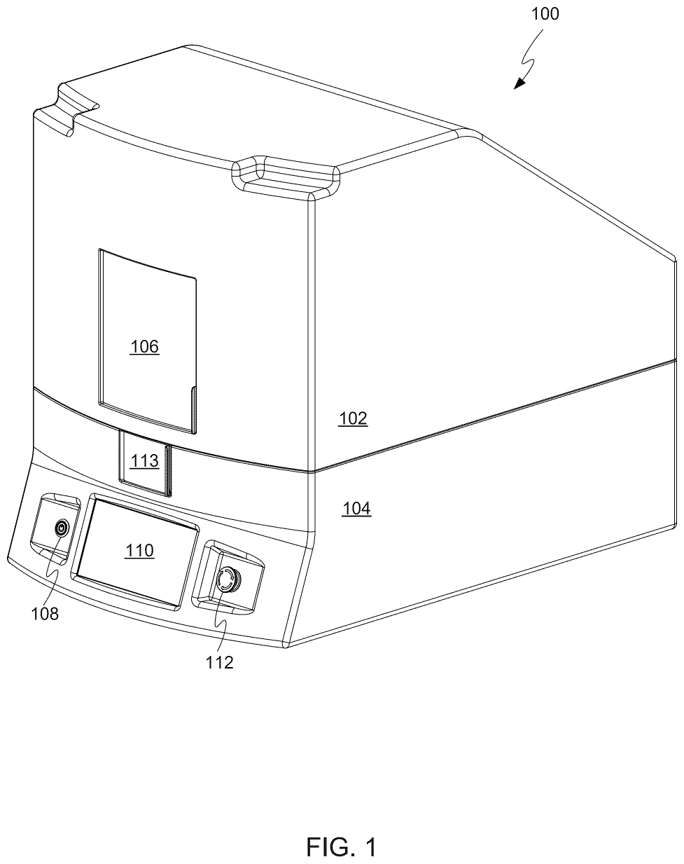

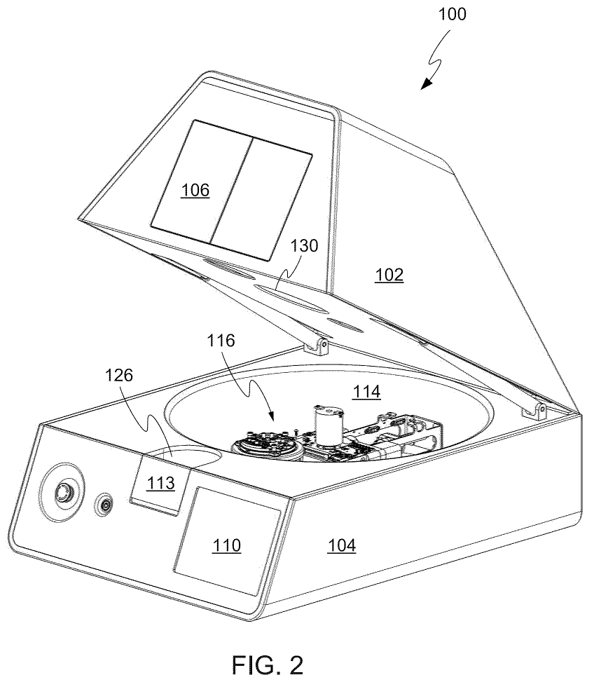

Turning now to the figures, FIGS. 1-2 show perspective views of a cell processing device 100. In particular, FIG. 1 shows a perspective view of cell processing device 100 in a closed state. Cell processing device 100 includes an upper housing assembly 102 hingedly attached to a lower housing assembly 104. Upper housing assembly 102 includes a first door 106 that allows for the insertion or removal of one or more cassettes into or out of upper housing assembly 102. First door 106 may be configured to slide or swing between its open and closed states. Lower housing assembly 104 can include one or more controls for manipulating settings of cell processing device 100. In particular, these one or more controls can include an on/off button 108, a touch sensitive electronic display 110 and an auxiliary control 112. In some embodiments, auxiliary control 112 may be configured to initiate a shut down or termination in operation of cell processing device 100. Touch sensitive electronic display 110 may also be configured to display multiple operating parameters associated with cell processing device 100 and may be configured to allow an operator to make changes to various operating parameters of cell processing device 100. Lower housing assembly 104 further comprises a second door 113 that allows for the insertion or removal of one or more vials from lower housing assembly 104 without separating upper housing assembly 102 from the lower housing assembly 104.

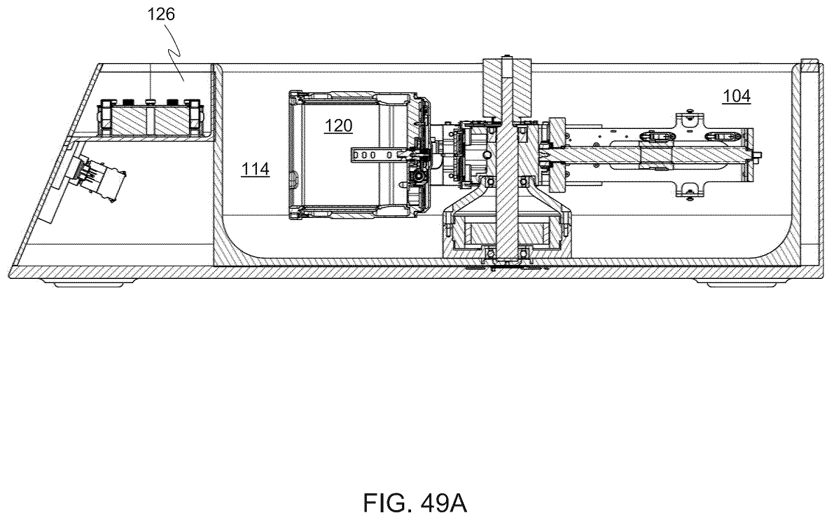

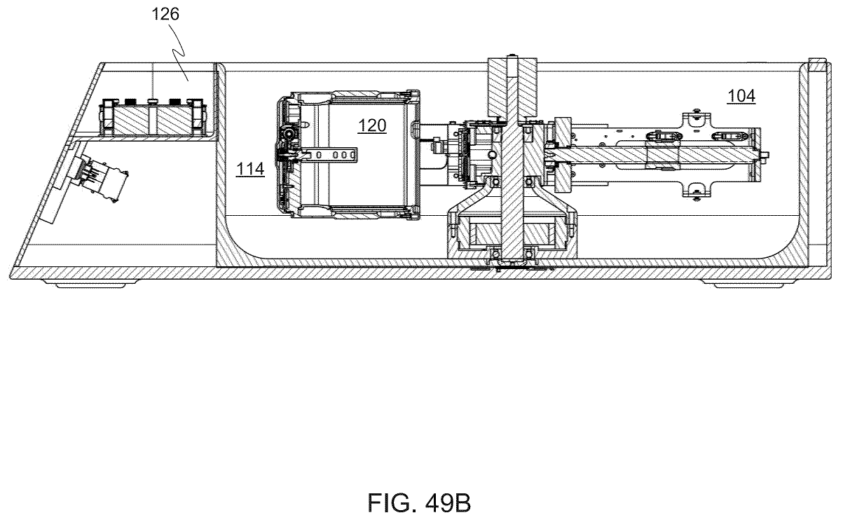

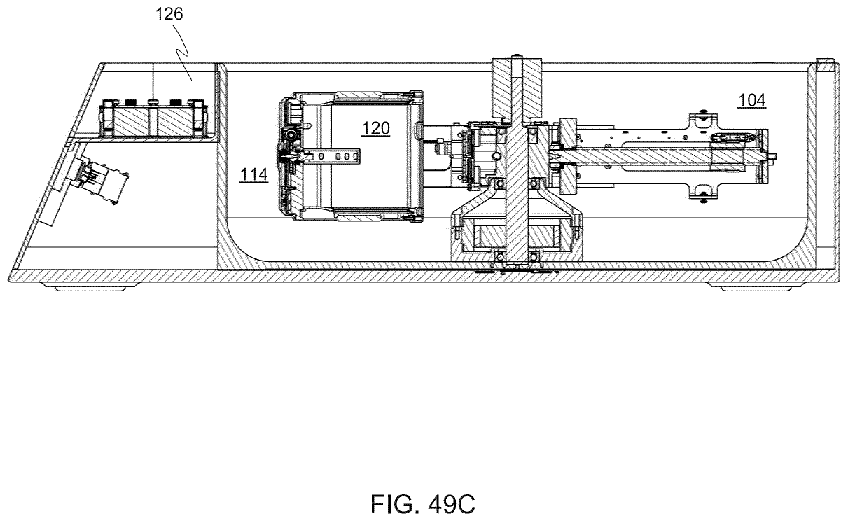

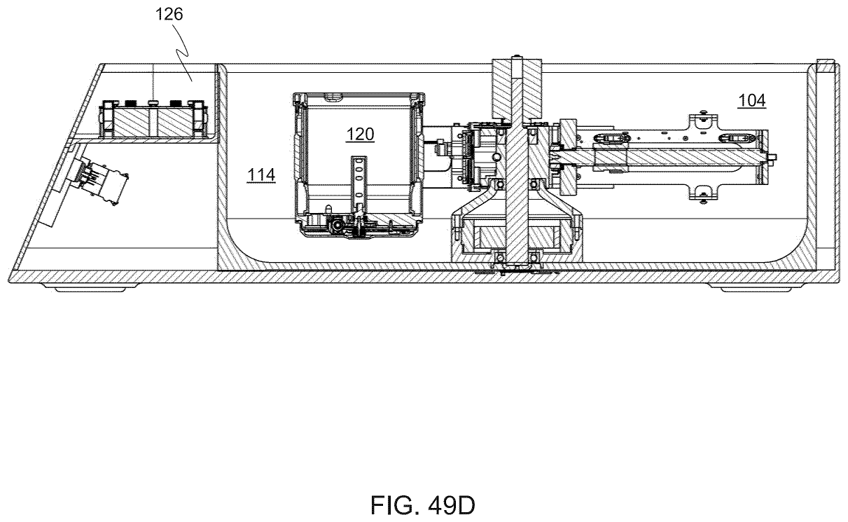

FIG. 2 shows upper housing assembly 102 rotated away from lower housing assembly 104 to allow for the removal or insertion of one or more disposable cassettes into or out of lower housing assembly 104. Lower housing assembly 104 further defines a centrifuge well 114 for accommodating operation of a processing device taking the form of a centrifuge 116. As depicted, centrifuge 116 may be a horizontal or swinging bucket type centrifuge that includes a rack or centrifuge bucket 120 for holding a sample container. Bucket 120 may be rotatably coupled to opposing ends of rotor yoke 122, as depicted. Rotor yoke 122 is rotated about an axis of rotation 124 during some operating modes of centrifuge 116. In some embodiments, a cowling or aerodynamic covering may be employed around the rotating components to minimize unnecessary air drag during centrifugation, enhance the operational efficiency of the system and optimizing energy usage during high-speed rotations An additional cross-sectional view is shown in FIGS. 49A-49D.

It should be appreciated that in some embodiments, the processing device may include a larger number of buckets for accommodating additional cassettes (e.g. processing cassettes 212 described below). For example, rotor yoke 122 could include additional buckets with an additional bucket that would allow for cell processing device to perform a larger number of concurrent operations. In an alternative embodiment, the platform could operate with two buckets and two counterweights, effectively running two independent processes simultaneously. For example, if the platform processes two leukapheresis collections—one containing 150 mL and the other 200 mL—an additional 50 mL could be added to the smaller collection to equalize the volumes at 200 mL each. This approach eliminates the need for a counterweight, as the identical processes running concurrently in the balanced buckets ensure system stability without additional adjustments.

Lower housing assembly 104 further comprises a vial cassette recess 126 to accommodate a cassette configured to hold multiple vials that can contain different reagents to assist in operation of cell processing device 100. In some embodiments, the cassette accommodated by vial cassette recess 126 may also include empty vials configured to receive material samples during operation of cell processing device 100. The material samples can help to confirm proper operation of cell processing device 100 and/or to calibrate subsequent operation of cell processing device 100. Upper housing assembly 102 includes through holes 128 and 130 through which cassettes positioned within upper housing assembly 102 can extend through to interact with cassettes positioned within vial cassette recess 126 and bucket 120. Lower housing assembly 104 also defines flask recess 127, which is configured to accommodate a flask for collecting waste materials.

In some embodiments, cell processing device 100 may include rubberized feet 101 that help to dissipate any vibrations transmitted through lower housing assembly 104 by rotation of centrifuge 116. In some embodiments the motor may be a stepper motor. In other embodiments, a customized centrifuge is utilized wherein the space between buckets is substantially filled, thereby minimizing air and therefore air resistance and noise as the centrifuge turns. In this embodiment, the shape may be contoured to allow the swiveling of the bucket and may largely fill the air space when the bucket is near horizontal, that is, when the centrifuge is at a high RPM.

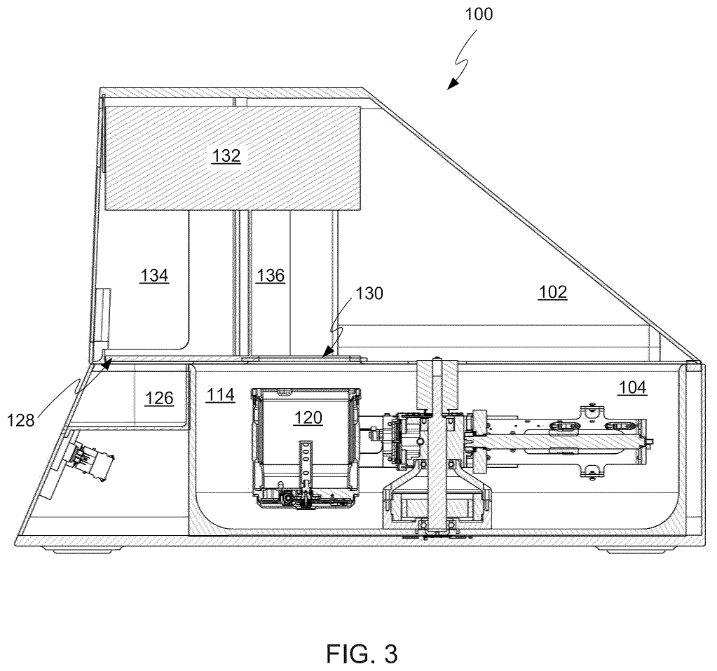

FIG. 3 shows a cross-sectional side view of cell processing device 100. Upper housing assembly 102 is shown including a cassette positioning assembly 132 that defines a syringe cassette recess 134 and a fluid cassette recess 136. Cassette positioning assembly 132 is configured to swap the position of cassettes positioned within recesses 134 and 136 in a Lazy Susan turntable manner. In addition to repositioning the cassettes positioned within recesses 134 and 136, cassette positioning assembly 132 may be further configured to affect rotation of cassettes around their own axis of rotation (not shown) within their respective recesses and to lower the cassettes at least partially into through holes 128 or 130 to allow for cassettes located within recesses 134 and 136 to interact with cassettes located in vial cassette recess 126 or bucket 120. In some embodiments, recesses 134 and 136 of cassette positioning assembly 132 may each include an iris allowing the cassettes to be lowered through holes 128 or 130, respectively. It should be noted that in some implementations cassette positioning assembly 132 may be configured to carry more than two cassettes. For example, a third cassette could take the form of a testing cassette used to confirm proper operation of a cassette disposed within centrifuge well 114. The testing cassette can allow for the cell processing device 100 to confirm proper operation of various consumables such as filters during and/or after various cell processing operations are performed.

FIG. 4 is a perspective view showing the relative positioning of cassettes to lower housing assembly 104. Upper housing assembly 102 has been removed to more clearly show the position of the cassettes relative to lower housing assembly 104. In particular, RSC 202 is shown positioned within vial cassette recess 126 directly below PFC 204. A waste tube 208 is shown and configured to receive waste produced during operation of the cell processing device. Waste tube 208 may be attached to PFC 204 and/or a TSC 210, which may each include a waste channel that allows for the extraction of waste material through a respective one of the cassettes. As depicted in FIG. 4, only PFC 204 is attached to waste tube 208. The PFC 204 stores various process fluids, such as buffers and culture media, required at different stages of cell processing. These fluids are automatically transferred to a Cell Processing Cassette (CPC) 212 as needed, supporting cell washing, sedimentation, and formulation. Removal of waste material from CPC 212 may be affected by routing pressurized air through a pneumatic channel that extends through PFC 204 and then into processing cassette 212, which in turn forces waste fluid out of CPC 212.

The waste-water port and fluid waste disposal system control liquid removal while maintaining target cell integrity and minimizing cell loss during cell processing. The waste-water port serves as a one-way outlet for fluid removal and is positioned centrally in the CPC lid. Its height and placement are configured to ensure that fluid can be removed efficiently while leaving behind a precise, minimum volume of liquid—15 mL—at the bottom of the sedimentation chamber when the CPC is upright and motionless. This design guarantees that target cells, which settle safely and are sequestered at the bottom of the sedimentation chamber, remain undisturbed during fluid extraction. The 15 mL volume left behind is consistent with the design intent to protect the target cell population during processing. The exact volume of residual fluid may vary slightly across multiple runs due to variations in initial fluid volumes or operational conditions. The fluid waste disposal tube is centrally located within the CPC and extends down into the cone to the 15 mL fluid level when the CPC is upright and static.

The waste disposal tube serves as the primary exit path for fluid removal, extending upward through the hollow center tube of the docked PFC and into an expandable, sealed waste container. Positive air pressure, introduced into the CPC through a hydrophobic filter in the lid, propels the fluid upward through the waste disposal tube. During this process, the CPC valve is rotated to one of the four park positions in the stator. This ensures that no other pathways exist for fluid to exit the CPC except through the centrally placed waste disposal tube. The central positioning of the tube also ensures even and controlled fluid movement, preventing turbulence or disruption of the cell sediment. The flow rate is regulated to avoid wicking up a clinically significant number of cells—99.9% of target cells remain undisturbed. This precision protects the integrity of the sequestered target cells while ensuring efficient waste fluid removal.

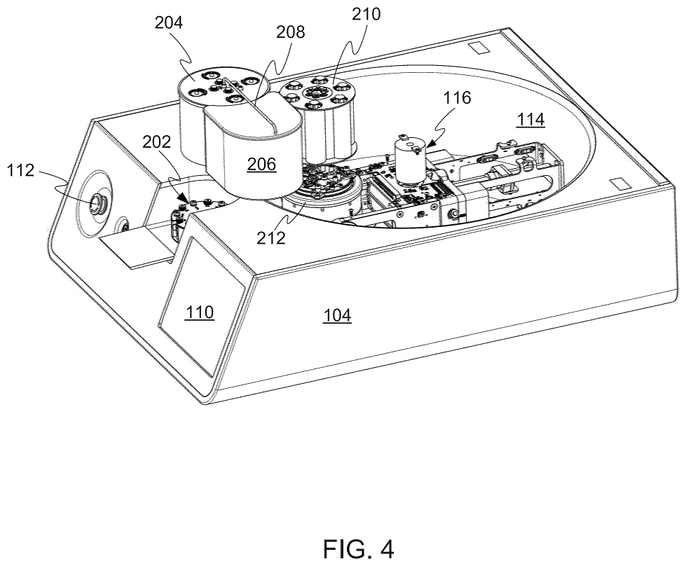

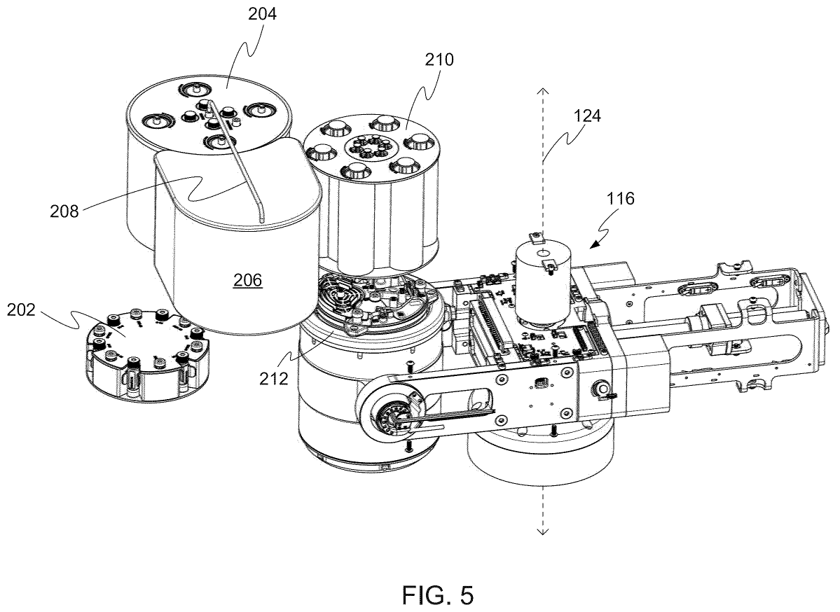

FIG. 5 shows a perspective view of all cassettes in the system according to a preferred embodiment, specifically showing RSC 202, PFC 204, TSC 210 and CPC 212. FIG. 5 also illustrates how bucket 120 is configured to rotate about axis of rotation 218 during operation of centrifuge 116.

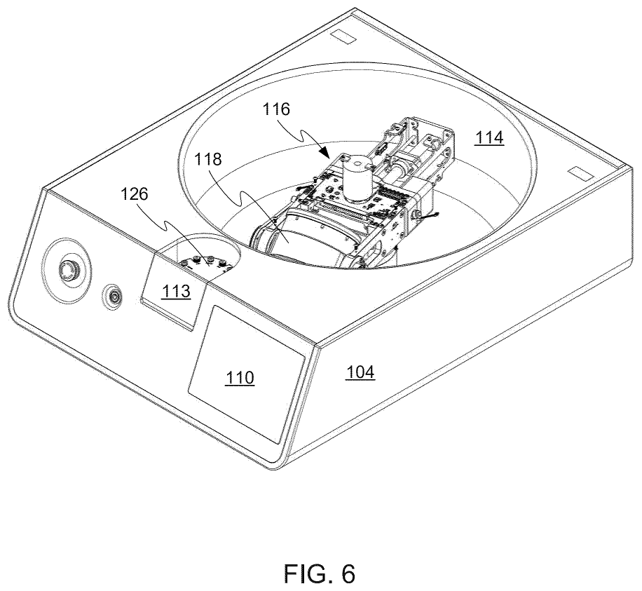

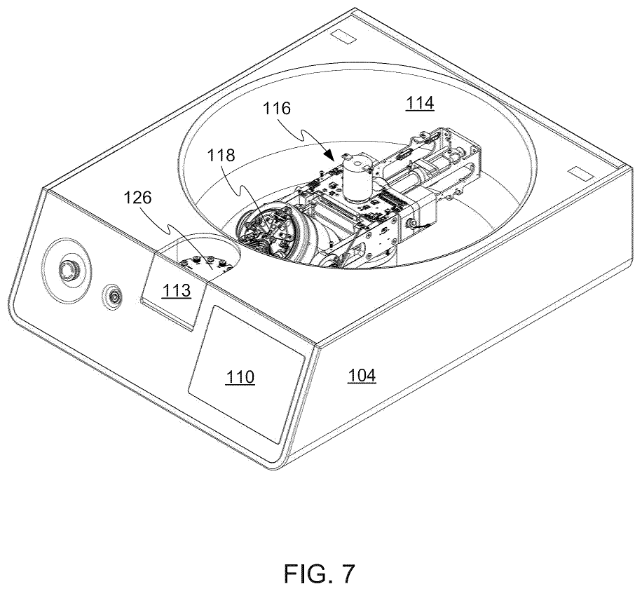

FIGS. 6-7 show two operating modes of centrifuge 116. Specifically, FIG. 6 shows a first operating mode in which centrifuge 116 operates in a centrifuge mode and FIG. 7 shows a second operating mode in which centrifuge 116 operates in a mixing mode. In the first operating mode shown in FIG. 6, a mixing motor associated with bucket 120 may be configured to tilt bucket 120 further so that a top face of processing cassette 212 faces directly toward axis of rotation 124. The depicted angle is typical of the angle achieved when exerting a force of about 50Gs on the sample contained within processing cassette 212.

FIG. 7 depicts centrifuge 116 operating in the second operating mode and consequently a bottom surface of bucket 120 is shown tilted toward axis of rotation 124. An angle of rotation as well as a speed of rotation of bucket 120 may vary in accordance with a desired mixing motion of sample material within processing cassette 212. Furthermore, bucket 120 will generally tilt toward and away from axis of rotation 124 while actively in a mixing operating mode. Generally, rotor yoke 122 will not turn around axis of rotation 124 and mixing will be limited to rotation of bucket 120 about axis of rotation 124. In some embodiments, rotor yoke 122 may be configured to turn much more slowly in the second operating mode than it does during the first operating mode. For example, a rotational speed of 10-20 RPM about axis of rotation 124 can in cooperation with rotation about axis of rotation 124 provide a more complex mixing mode.

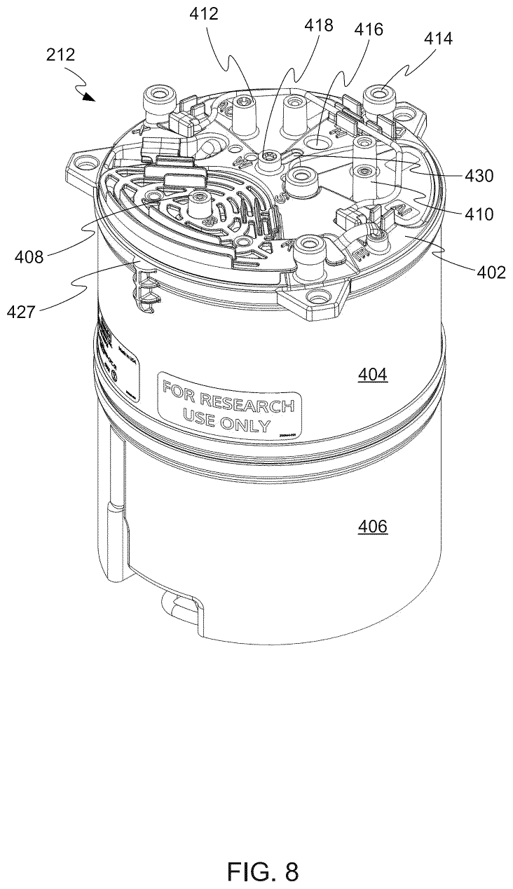

FIG. 8 is a perspective view of CPC 212. In particular, processing cassette generally includes a cover 402, upper housing component 404 and lower housing component 406. The cover 402 includes multiple ports for assisting in introducing and withdrawing sample materials from processing cassette 212.

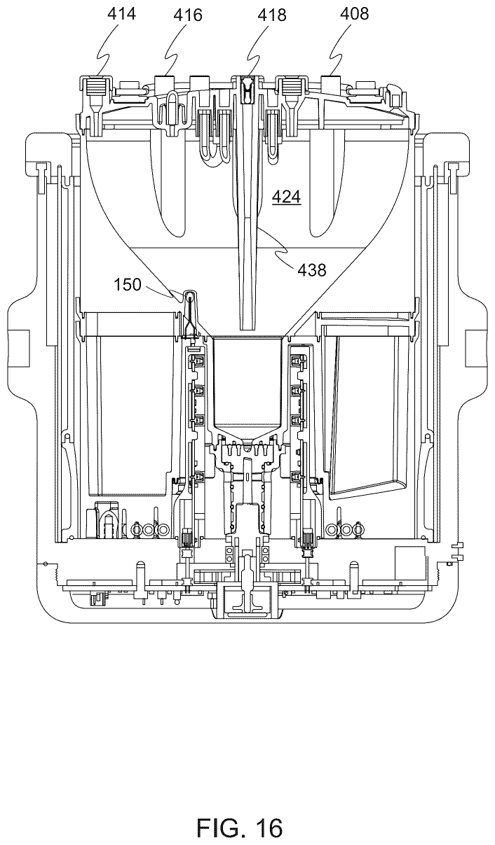

The CPC ports include a fluid port 408 (FIT Receptacle 3) for receiving culture media and buffer solution from PFC 204. Fluid port 408 may include a one-way valve, taking the form of a check valve, that allows fluid to enter into processing cassette 212 through fluid port 408 only when fluid port 408 mates with a port on PFC 204. Cover 402 further comprises pneumatic ports 410 and 412. Pneumatic outlet port 412 (FIT Receptacle 1) allows for the expulsion of pressurized air from processing cassette 212 to reduce or equalize air pressure within processing cassette 212. Pneumatic inlet port 410 (FIT Receptacle 2) allows for the introduction of air into processing cassette 212 to increase air pressure within a main chamber 424 (see FIG. 16) of processing cassette 212. The details of the air pressure control system will be described below. Pneumatic ports 410 and 412 can also include a check valve and the check valve within pneumatic outlet port 412 is oriented in an opposite direction from the check valve within pneumatic inlet port 410. An alignment fin 427 for aligning processing cassette 212 within bucket 120 is shown at bottom.

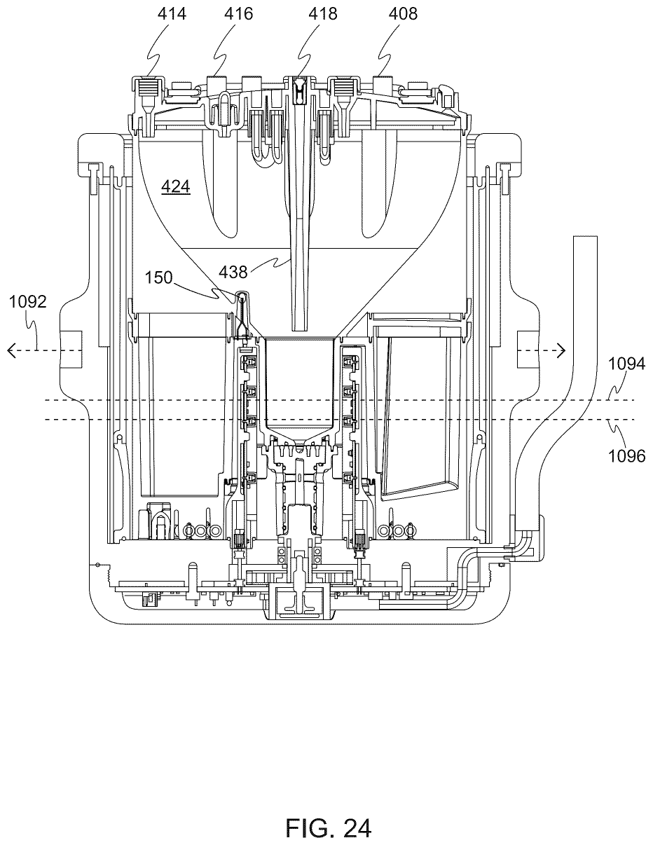

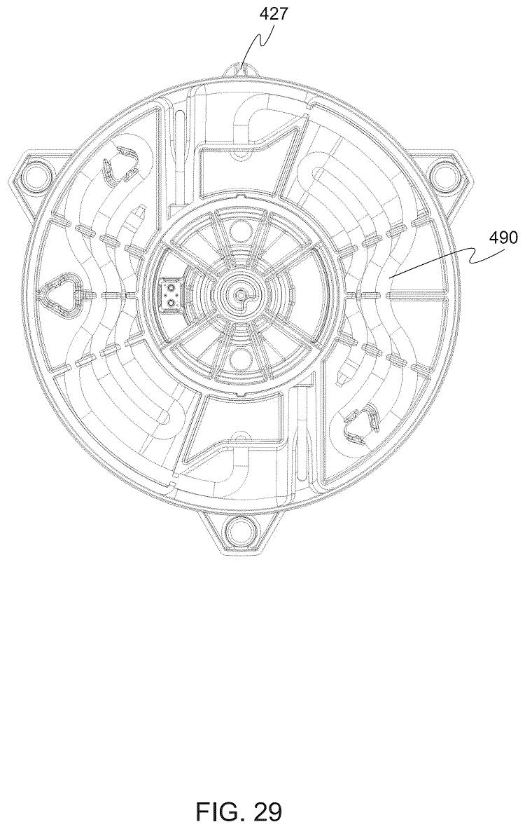

Cover 402 further comprises septa 414 and 416 (see for instance FIG. 24), which are configured to interact with and receive fluid supplied by injectors of TSC 210 or allow for collection of fluid by extractors of TSC 210. Specifically, the septa include harvest septum 414 and dissolved O2 well septum 416. All syringes within TSC 210 are sanitized prior to use and only used for one fluid transference operation each. In some embodiments, the syringes within TSC 210 may be concurrently sanitized by radiating the entire TSC with the syringes disposed within TSC 210. Upward facing surfaces or septa may be aligned in a single plane to reduce a complexity of sanitizing septa 414 and 416 prior to engaging septa 414 and 416 with a needle of an injector. Cover 402 includes a waste port 418 through which waste fluids can exit processing cassette 212 when pressurized gas (e.g. air, nitrogen, or carbon dioxide) is introduced into processing cassette 212 using pneumatic inlet port 410 and all other exits are restricted. Cover 402 further comprises an input tubing port 420 that facilitates the introduction of a blood sample into processing cassette 212 prior to, or after insertion of processing cassette 212 into cell processing device 100. Finally, cover 402 may include emergency recovery tubing 490 and a fluid level laser window 430 that detects the fluid level inside, preferably to an accuracy of 1 mm. The recovery tubing 490, shown again in FIG. 29, provides a backup means for recovering fluids or materials from the CPC during processing. The fluid level laser window 430 allows fluid volume and weight sensing that allows for real-time monitoring of fluid levels and weight, aiding in accurate volume management and quality control.

The following features describe this multiple port configuration in detail:

The CPC 212 includes multiple ports including the fluid port 408 and the pneumatic ports 410 and 412, at the top section of the cover 402 for fluid and gas transfer. The internal openings of these ports are positioned in a straight line along the uppermost region of the interior of the CPC 212. This in-line alignment ensures that when the CPC 212 tilts or rotates, the fluid level can rise up the walls of the tapered structure of main chamber 424 to its maximum allowable angle without reaching any port opening, preventing unwanted fluid entry into any of the ports.

To facilitate fluid injection, removal, and gas exchange, the CPC requires access points that may not align with the in-line configuration on the inside of the CPC lid. To achieve their purpose, the external locations of the ports are located radially in precise distances from the geometric center of the lid to be compatible and dockable with the automated TSC or PFC. Collectively the external ports are positioned to allow docking by either the TSC or the PFC by positioning either cassette directly over, and centered, on the CPC lid needing only a precise rotation and lowering to dock their exit ports with the appropriate outlet port of the CPC lid.

Between the external ports and their respective in-line internal openings, fluid pathways are constructed to direct each port to its designated in-line position inside the CPC. These pathways bridge the offset positions of the external ports with the uniform, in-line configuration on the inside. This arrangement allows the system to access each port externally without disrupting the internal alignment that prevents fluid from contacting the port openings during cell processing movements.

This in-line internal port configuration provides several functional advantages. First, by preventing fluid from reaching the ports, the CPC can tilt to a wider range of angles, maximizing mixing and fluid dynamics without the risk of unwanted fluid entry. Second, it provides sterile access for syringe mechanisms. The offset external ports enable syringe or other transfer mechanisms to access the CPC without compromising the safety of the internal in-line configuration. Third the fluid pathways ensure that only designated fluids or gases can enter the CPC through controlled pathways, maintaining aseptic conditions and preventing unintended mixing

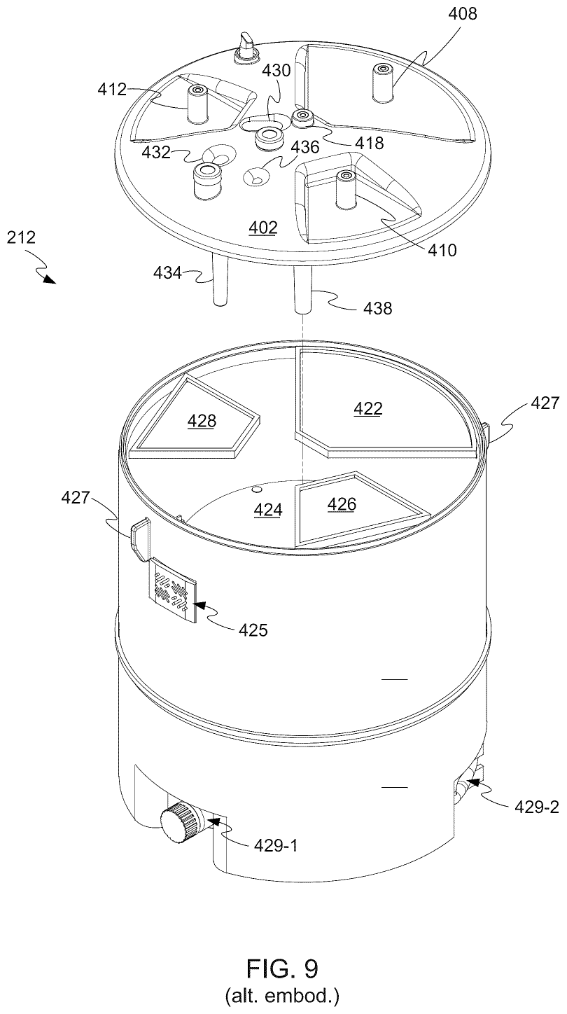

FIGS. 8 and 9 also show a vent assembly 425 located on an exterior surface of upper housing component 404. Vent assembly 425 includes vents that may be configured to facilitate venting of air located within fraction chambers disposed within lower housing component 406. This prevents the addition of solution from main chamber 424 into the fraction chambers from causing unwanted pressure buildup within the fraction chambers. Alternatively, processing cassette 212 may comprise vents that empty out into an area between cap 402 and filter 426 or 428 that prevent any contaminates traveling through the vents from entering into main chamber 424.

FIGS. and 9 show an alternative embodiment partial exploded view of processing cassette 212 with cover 402 removed from upper housing component 404 to reveal multiple filters disposed in a top region of upper housing component 404. In particular, a hydrophilic filter 422 is a largest one of the filters. Hydrophilic filter 422 is positioned beneath fluid port 408 and configured to prevent any contamination present in the fluid introduced through fluid port 408 from getting into and contaminating a main chamber 424 of processing cassette 212 located just beneath hydrophilic filter 422. The large area and horizontal orientation of hydrophilic filter 422 allows fluid introduced into processing cassette 212 through fluid port 408 to spread evenly across hydrophilic filter 422 in order to pass through it. In some embodiments, hydrophilic filter 422 may have a pore size of about 0.2 microns, which is operable to clean impurities from buffer or culture media solution being introduced through fluid port 408. Processing cassette 212 further comprises hydrophobic filters 426 and 428 that allow for the passage of air and/or other gases into and out of processing cassette 212 using pneumatic ports 410 and 412. While filters 422, 426 and 428 are shown positioned within upper housing component 404 to represent their position when processing cassette 212 is closed up, these filters are secured to an interior facing surface of cover 402 to prevent escape of contaminates located between filters 422, 426 and 428 and the interior facing surface of cover 402. Alternatively, filters 422, 426 and 428 could be attached and/or sealed to an exterior-facing surface of cover 402 in which case ports 408, 410 and 412 could be packaged with respective filters 422, 426 and 428 to form exterior filter assemblies.

Cover 402 is also depicted including multiple indentations. In particular, fluid level laser window 430 is positioned directly below a laser sensor positioned on an exterior facing surface of cover 402 and configured to measure a volume of fluid within a main chamber 424 of processing cassette 212. Indentation 432 is directly above a pH probe 434 that is configured to monitor a pH of sample material disposed in said main chamber 424 of processing cassette 212. Indentation 436 is directly above a dissolved oxygen sensor that is helpful in confirming an amount of dissolved oxygen within the main chamber of processing cassette 212 is maintained at a desired level during operation of cell processing device 100. Cover 402 further comprises a waste standpipe 438, which provides a path by which waste solution within CPC 212 may be evacuated through waste port 418.

Alignment fins 427 are distributed around a periphery of upper housing component 404 and interact with grooves in bucket 120 to radially align processing cassette 212 with bucket 120. This ensures proper alignment of openings in a downward facing surface of processing cassette 212. Lower housing component 406 further comprises drains 429-1 and 429-2 that accommodate removal of material from fraction chambers located within lower housing component 406.

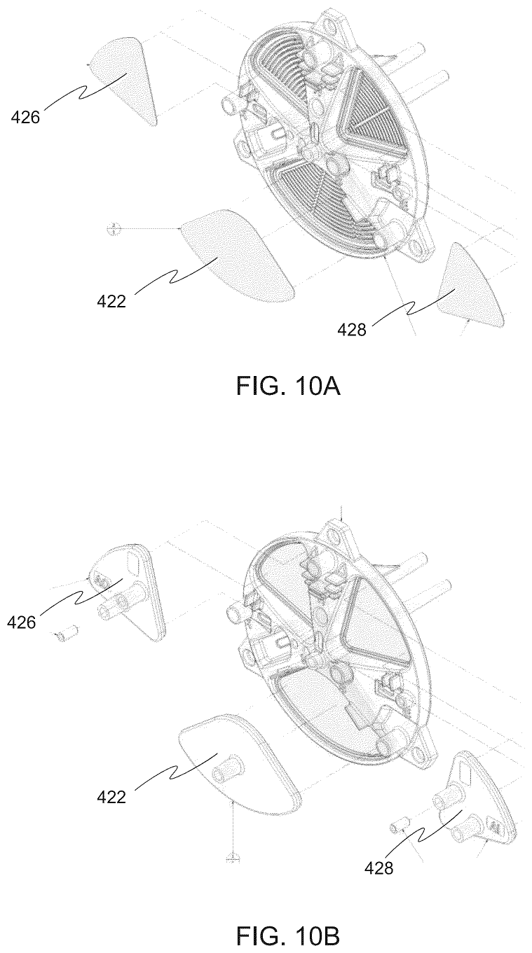

FIG. 10A and FIG. 10B show the three filters 422, 426 and 428 disposed in the top region of upper housing component 404.

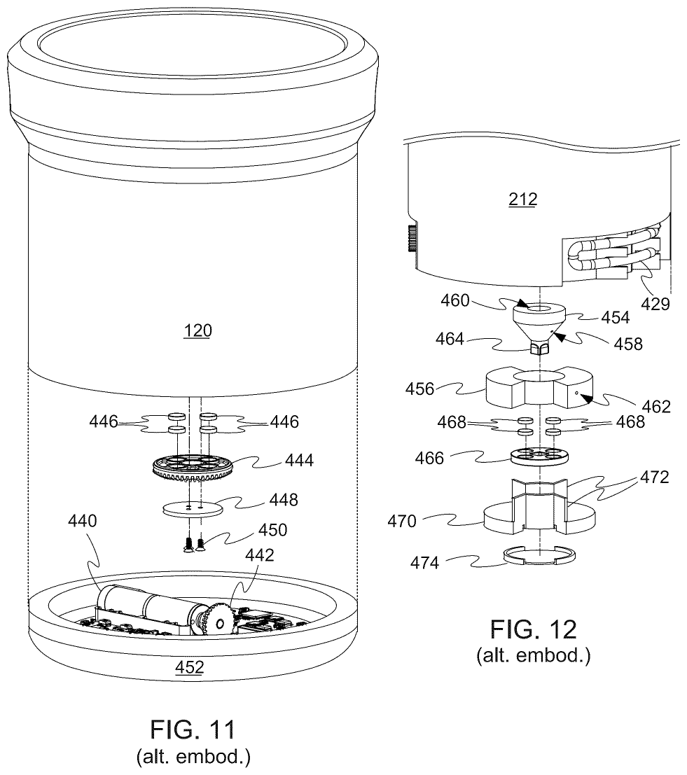

FIG. 11 shows a partial exploded view of an alternative embodiment of bucket 120. In particular, FIG. 11 shows a magnetic valve actuator. The magnetic valve actuator includes a motor 440 configured to turn a shaft having a distal end attached to a first gear 442. First gear 442 is configured to engage teeth on second gear 444, which is oriented orthogonally relative to first gear 442 forming what is sometimes referred to as a miter gear configuration. It should be noted that straight, spiral, zerol, miter or hypoid gear configurations are also possible. Consequently, rotation of first gear 442 by motor 440 results in rotation of second gear 444. Second gear 444 includes multiple recesses for accommodating the placement of magnets 446. Magnets 446 are generally rare-earth magnets formed from materials such as Neodymium or Samarium Cobalt. After placing magnets 446 within recesses of second gear 444, rotation of second gear 444 generates a rotating magnetic field capable of rotating a valve positioned at a bottom end of CPC 212. Other means of rotating the valve may be employed.

FIG. 12 shows an exploded view of a harvest valve assembly that includes a first valve segment 454 and a second valve segment 456. First valve segment 454 may be configured to rotate about its longitudinal axis relative to second valve segment 456, which is configured to remain stationary. First valve segment 454 defines a first channel segment 458 configured to guide solution received in a tapered recess 460 arranged at a first end of valve segment 454 into one of multiple channel segments defined by second valve segment 456. While only a second channel segment 462 is depicted in FIG. 12 it should be appreciated that second valve segment 456 can include multiple channel segments with at least one channel segment for guiding sample materials to each of multiple fraction chambers arranged around a lower periphery of processing cassette 212. First valve segment 454 further comprises a non-circular second end, opposite the first end that is configured to engage a central opening in a magnet carrier 466. The central opening can have a shape and size matching the shape and size of non-circular second end 464 of first valve segment 454. While second end 464 is depicted as being roughly square in shape, the important aspect is that the geometry allows the central opening of magnet carrier 466 to impart a force on second end 464 sufficient to rotate first valve segment 454 relative to second valve segment 456.

Magnet carrier 466 is configured with peripheral recesses sized to accept multiple magnets 468 and formed from a non-magnetic material. In this context non-magnetic material refers to materials such as plastic or ceramic and mostly magnetically neutral metals such as aluminum. Use of a non-magnetic material prevents interference with a magnetic coupling between magnets 468 and magnets 446. In this way, rotation of magnets 446 in second gear 444 causes rotation of the magnets 468, which then imparts a force to first valve segment 454 sufficient to align channel segment 458 with a channel segment defined by second valve segment 456 when it is time to drain sample material from main chamber 424 of processing cassette 212. While magnets 468 are described as magnets it should be appreciated that in some embodiments, magnet carrier 466 could instead carry magnetically attractable elements formed from ferritic material that would still be capable of responding to a shifting magnetic field generated by rotation of the magnets carried by second gear 444. Magnet carrier 466 sits in a circular opening defined by a base of a valve segment carrier 470. Valve segment carrier 470 is sized to also receive second valve segment 456 and includes vertical walls 472 that prevent rotation of second valve segment 456. The harvest valve assembly further comprises a non-magnetic cover 474 that prevents scratching of an interior of bucket 120 during rotation of magnet carrier 466.

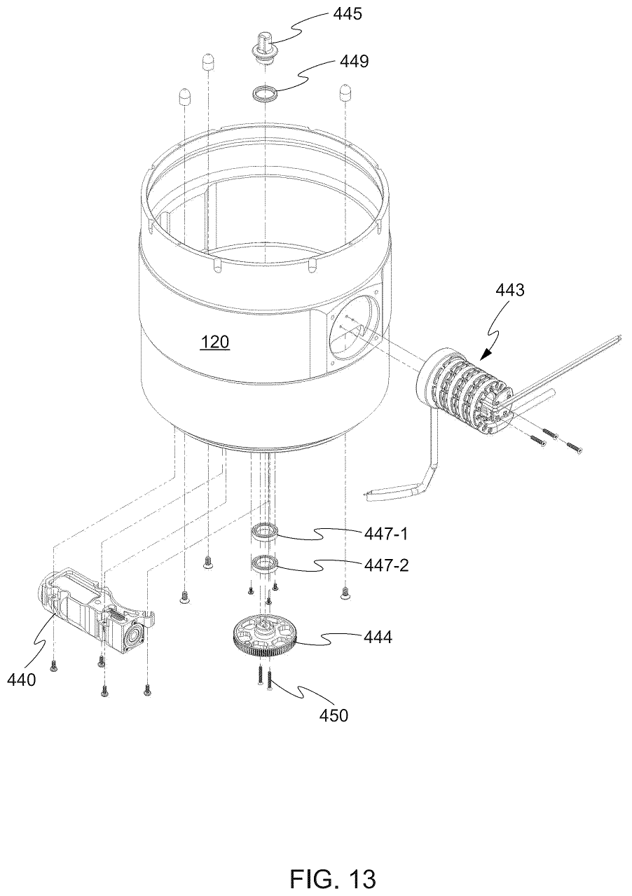

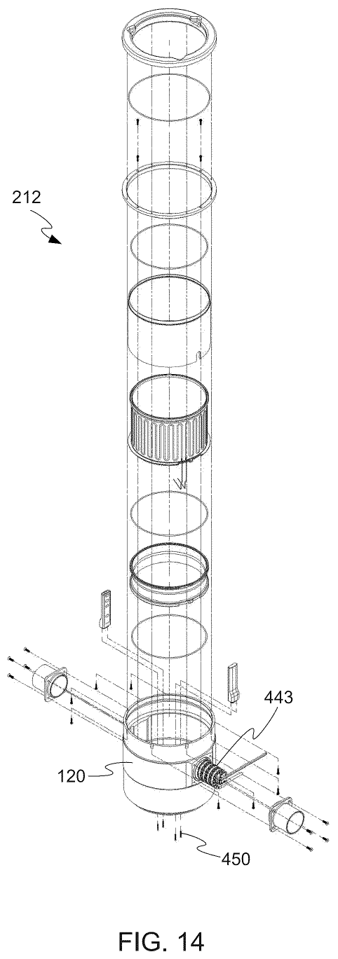

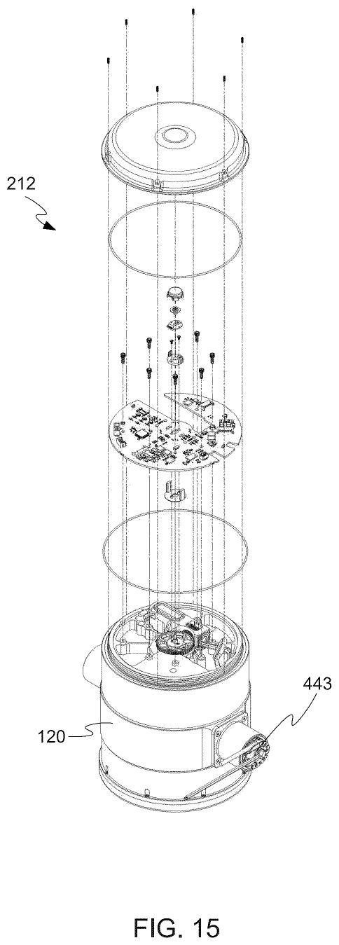

FIGS. 13-15 show exploded views of the centrifuge bucket 120 of the preferred embodiment, where in particular, bucket 120 could instead include a modified second gear 444 that is coupled to a drive shaft 445 by a plurality of fasteners 450. Bearing elements 447-1 and 447-2 are positioned within a through hole extending through a bottom wall of bucket 120 and assist with rotation of second gear 444 and drive shaft 445 within the through hole. Any liquid inadvertently escaping the processing cassette may be prevented from entering into the recess housing motor 440 by environmental seal 449, which fits around a base of drive shaft 445. FIG. 13 also illustrates how a distal end of drive shaft 445 is non-circular. In particular, the distal end of drive shaft 445 can have a D-shaped or crescent-shaped distal end.

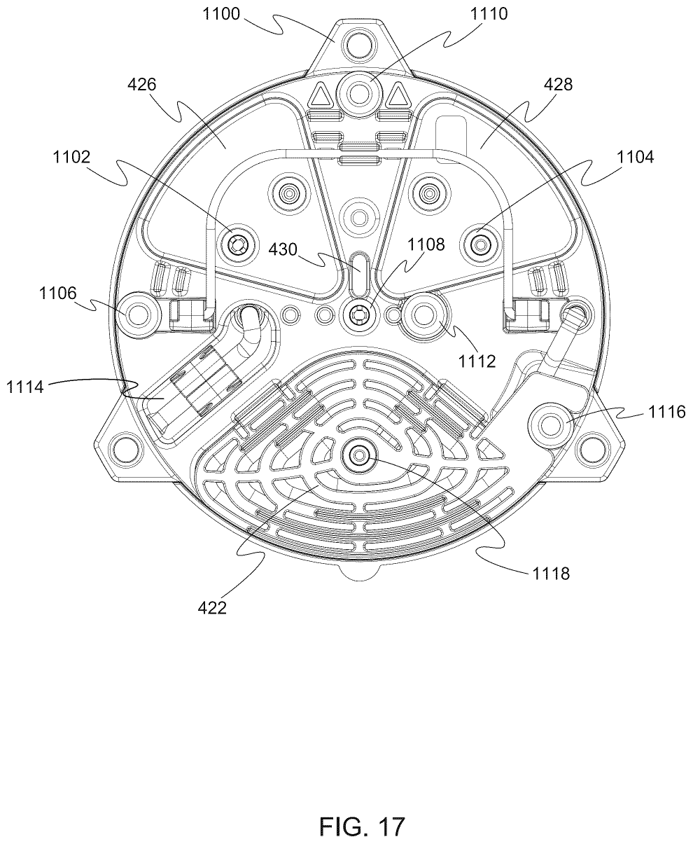

As shown in FIGS. 16 and 17, the top of the CPC 212 is configured with multiple input and output ports, each serving a critical role in maintaining a sterile and controlled environment for CAR T-cell manufacturing. These ports allow for the efficient, aseptic transfer of various fluids, gases, and samples thereby supporting the entire cell processing cycle, from initial input to final harvest. Each input port is equipped with sterile filters or needle septa which are UV disinfected before each use to ensure aseptic entry or extraction and fluids to prevent contamination and to maintain the purity of the internal environment. This strategic arrangement of input and output ports, along with integrated filtration systems, enables the ACP to conduct complex cell processing operations in a closed, sterile environment, supporting high-purity CAR T-cell production with minimized contamination risk. As shown in FIG. 17, there is a sealed stub of tubing 1114 from blood or leukapheresis bag.

The CPC 212 includes dedicated input ports for gases-in 1104, fluids-in 1118, reagents-in 1116. Gases in extend through hydrophobic filters 426 and 428, which allow the introduction of sterile gases as needed for maintaining cell culture conditions or controlling internal pressure during certain processing stages. Fluids port 1118 enables the addition of various fluids required for cell processing, such as washing solutions, culture media, or transduction agents into the CPC. Fluids in are through hydrophilic filters 422. Reagents-in 1116 provides access for the controlled introduction of specific reagents, including those needed for cell activation or genetic modification steps.

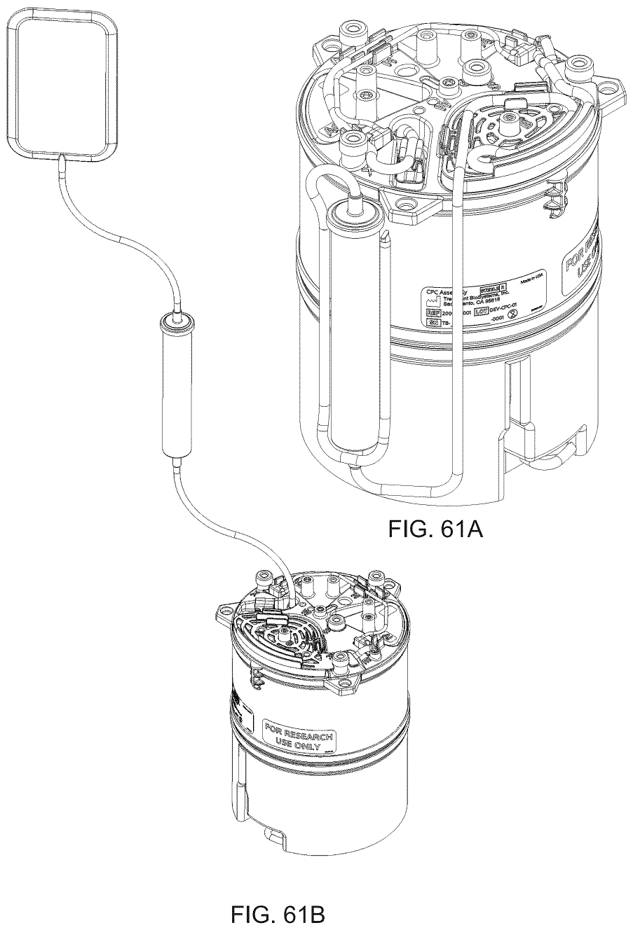

The CPC 212 includes a sealed stub of tubing 1114 from a blood or leukapheresis bag. The tube is shown again in coiled and uncoiled form in FIGS. 61A and 61B, respectively. The patient's cells enter through this port for processing. A clot filter in this tube prevents clots from passing. The bag with the cell sample from the patient is tube-sealed to this port.

The CPC 212 also features output ports for harvesting CAR T-cells-out 1110, Sterile air-access port 1106, gases-out 1102 and fluids-out 1108 and samples-out 1112. The output port 1110 is dedicated to the final extraction of the resulting CAR T-cell product following completion of the cell processing steps. The sterile air-out 1102 facilitates controlled release of gases to maintain internal pressure and prevent unwanted accumulation of air within the cassette 212. Through gases-out 1102, air is released through a hydrophobic filter 426 ensuring any gas leaving the system is sterile. Gases-out 1102 and fluids-out 1108 allow for the safe removal of gases and fluids from the CPC 212 during washing, transduction, and other intermediate stages. CAR T-cells-out port 1110 is dedicated to the final extraction of the CAR T-cell product following completion of the cell processing steps. Samples-out 1112 provides a pathway for collecting cell samples or aliquots at different stages, enabling quality control testing or process monitoring without disturbing the internal environment.

The ports of the CPC 212 are equipped with specialized filters such as 0.2 μm hydrophobic filters 426, 428 and 0.2 μm hydrophilic filter 422 to support sterility. The 0.2 μm hydrophobic filters 426, 428 are used at various gas ports to ensure sterile air entry or exit thereby preventing contamination from external particles. The 0.2 μm hydrophilic filter 422 positioned beneath the coiled tubing allows for sterile fluid flow and ensures aseptic conditions are maintained across all fluid transfers.

The wider top section of the CPC accommodates multiple ports for fluid and gas transfer. These ports, collectively, allow for the aseptic introduction of fluids, such as cell suspensions, reagents, or culture media, and the removal of waste fluids and retrieval of formulated volume of gene-modified cells. The inclusion of gas exchange ports also provides aseptic introduction and removal of gases within the CPC, maintaining sterile conditions and supporting processes that may require different levels of oxygen or carbon dioxide.

The CPC's circular lid design enables precise docking alignment through use of a number of alignment features 1100 to mate with other system components, such as the PFC and the TSC. The lid of the CPC features multiple liquid transfer ports (septa) designed to enable precise, aseptic fluid handling and improve overall cell processing efficiency

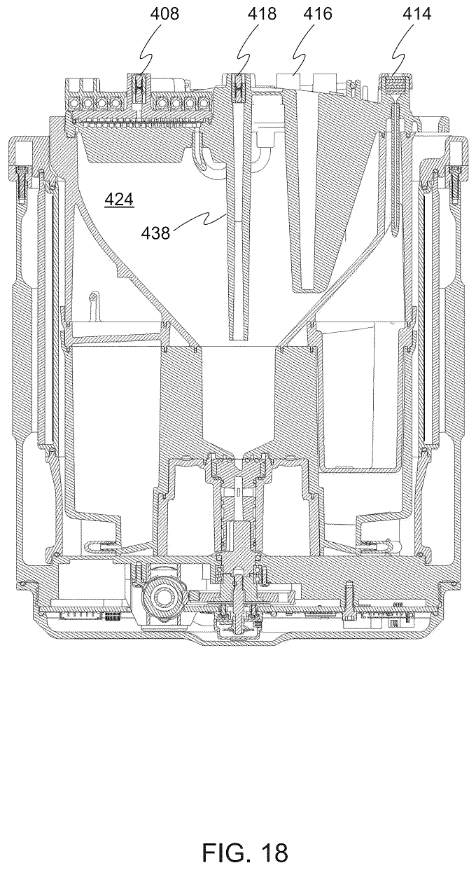

FIG. 18 shows a cross-sectional side view of an alternative embodiment of the invention of CPC 212 disposed within bucket 120. In particular, FIG. 18 shows how in this embodiment, fastener 450 couples second gear 444 directly to drive shaft 445 and how environmental seal 449 prevents the intrusion of any fluids in bucket 120 from passing into and disrupting operation of motor 440. The distal end of drive shaft 445 is shown protruding into a recess defined by first valve segment 454.

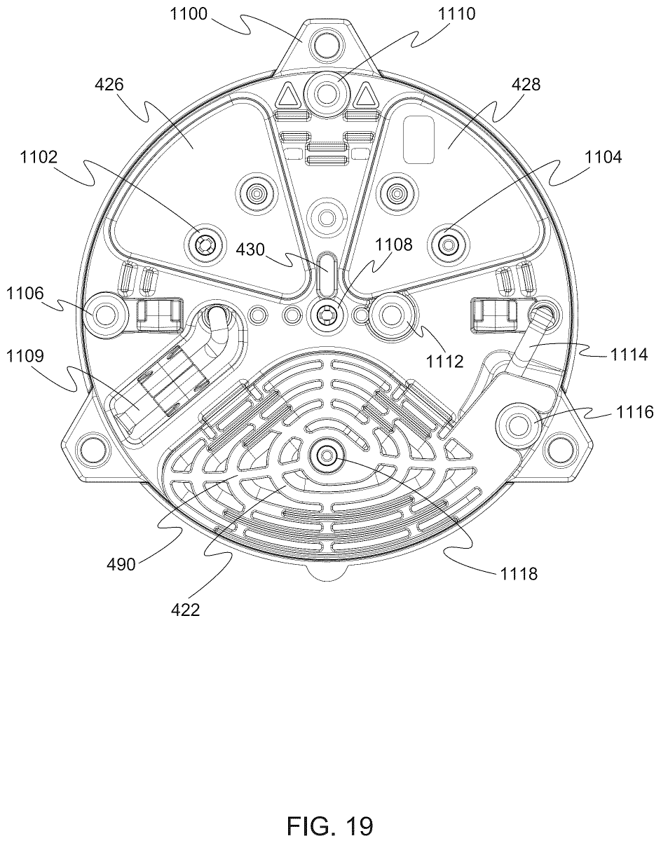

FIG. 19 shows a plan view of CPC 212 according to a preferred embodiment of the invention.

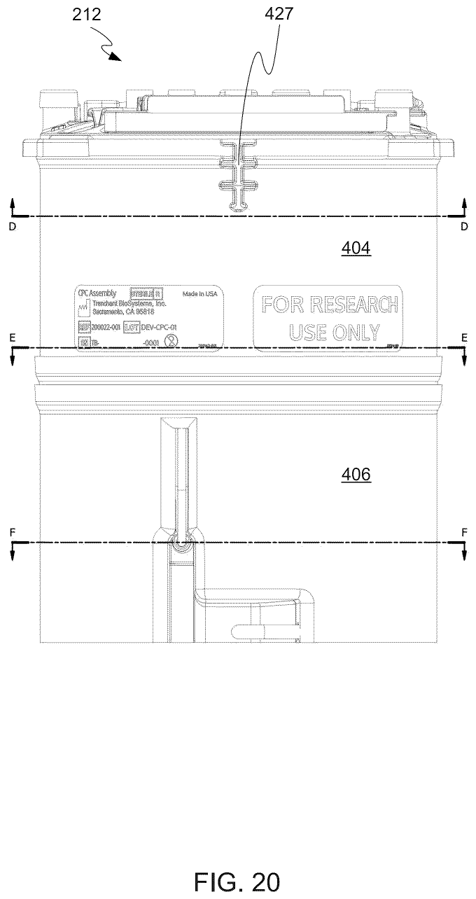

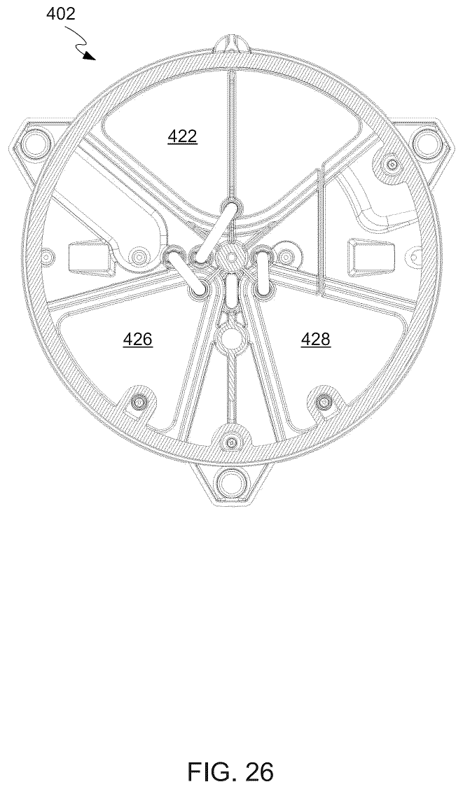

FIG. 20 shows a side view of CPC 212 as well as section lines D-D, E-E, and F-F, which are shown in more detail in FIGS. 26, 27 and 28, respectively;

Section line D-D is a view upwards to the filter configuration and sensor configuration arranged directly beneath cover 402. Section line E-E is a downward view into the main chamber 424 of CPC cassette 212 and section line F-F cuts through fractional chambers defined by a lower periphery of CPC 212.

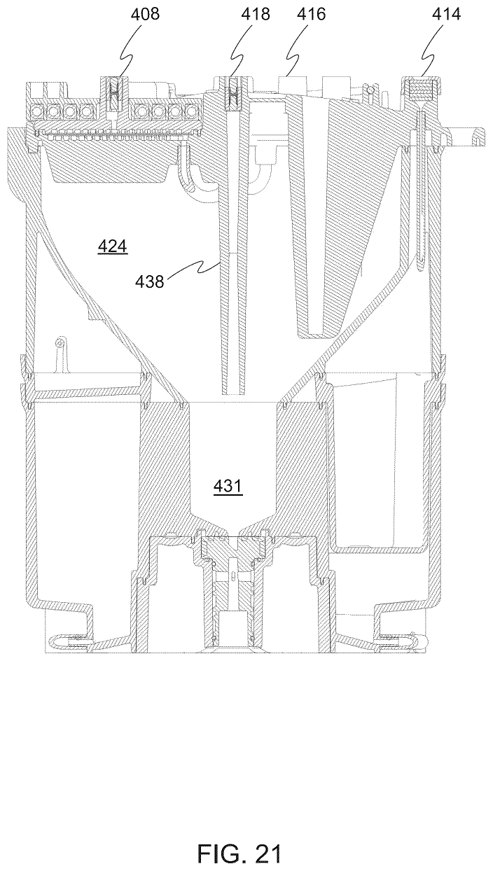

FIG. 21 shows a cross-sectional side view of CPC 212. Main chamber 424 may be configured to hold about 350 mL of fluids, however, it should be appreciated that the size of main chamber 424 may vary depending on how large a blood sample is being processed and what dilution level for the blood sample is desired. FIG. 21 shows waste port 418 including a first check valve that allows for the passage of fluid out of main chamber 424 and prevents fluids from entering into main chamber 424. FIG. 21 also depicts how fluid port 408 includes a second check valve oriented in an opposite direction from the first check valve that allows for fluids to enter into fluid port 408 but prevents fluids from exiting fluid port 408.

The CPC 212 features a tapered, cone or elliptic cone shape that narrows progressively from a wider diameter at the top to a narrower diameter at the bottom, wherein the ratio of diameters is preferably between 4 and 5, in less preferred embodiments lower than 4. This design provides a range of functional advantages: The tapered, conical or elliptical interior geometry of the CPC plays a critical role in achieving precise cell concentration, sedimentation, and mixing.

During centrifugation or sedimentation processes, the tapered shape of main chamber 424 naturally directs sediment—such as target cells, including hematopoietic stem and progenitor cells, T-cells, or NK cells—toward the narrow bottom section of the CPC 212, referred to as the sedimentation column 431 (see FIG. 18). The sedimentation column features substantially vertical, cylindrical walls that enhance measurement accuracy. This geometry focuses sediment (preferably cells) into a narrow area, improving sensitivity for optical detection. In one embodiment, the sedimentation column 431 is designed with flat vertical surfaces wide enough to accommodate the light beam emitted from an optical emitter to a sensor.

The optical detection system for cell monitoring in the CPC 212 utilizes a plurality of optical emitter/detector pairs positioned along the sedimentation column to measure variations in light transmission caused by the presence, movement, and distribution of cells within the suspension. The system supports light across visible and non-visible wavelengths (e.g., infrared or ultraviolet), with visible light currently in use. The system is adaptable to accommodate non-visible wavelengths, depending on specific requirements for improved sensitivity, reduced interference, or the optical properties of the sample or reagents. The detection system operates through a configurable sequencing scheme to optimize cell detection, sedimentation monitoring, and real-time feedback during cell processing operations. The sequencing algorithm includes the following features.

The plurality of optical emitter/sensor pairs is at least two, preferably three, and in some embodiments more than three pairs to detect light signals through the sedimentation column of the main chamber of the CPC. Although most of the main chamber of CPC is funnel-like and with a curved wall, the sedimentation column walls are essentially vertical and parallel and include flat portions through which the emitter-sensor pairs operate, the flat portions minimizing disrupting reflections that would be caused were those surfaces curved. The flat section of the sedimentation column is on both the emitter and sensor side. Preferably, the wavelength is an adjustable white light to allow it to best detect all cells and particularly white cells during various stages of system operation.

The intensity of each emitter is configurable to optimize sensitivity and detection performance. The system can operate at absolute intensities (comparing detected light intensity against predefined thresholds to identify cell presence or movement) or utilize relative intensities (monitoring changes across the array to determine sedimentation dynamics, cell layering, and distribution), accounting for fluid dynamics, optical path lengths, and variations in cell density. The system may also utilize changes in the color of the transmitted or received light as an additional parameter to measure cell density or identify specific characteristics of the cell suspension. It is known that cells or particles in a suspension can scatter, absorb, or reflect light differently depending on their density, size, or type. The color (wavelength) of the light that passes through or is detected by the optical emitter/receiver pairs may change based on how the light interacts with the suspension. As cell density increases, the fluid's opacity changes, but the spectrum (color) of transmitted or scattered light can also shift. For example, densely packed cells may absorb more light in specific wavelength ranges (e.g., red or blue), causing a measurable color shift in the detected signal. By analyzing both light intensity (opacity) and color variation, the system can enhance sensitivity to small changes in cell density; and potentially differentiate between cell types (e.g., red blood cells, T-cells, or NK cells) based on unique optical absorption characteristics. This provides a more robust and nuanced detection mechanism for cell concentration or composition. In summary the optical emitter could use multi-wavelength light sources (e.g., white light or specific RGB LEDs), the receivers could detect intensity, and wavelength shifts to measure color variation caused by changes in the suspension. The system's algorithm may incorporate these readings into its cell count or concentration calculations.

The plurality of optical emitter-sensor pairs allows for real-time monitoring and control of cell layering and density, which is essential for precise cell recovery and distribution. For instance, the data from the optical emitter-sensor pairs may be used to control centrifuge RPM, for instance the speed of the centrifuge could be correlated to the level of packing of the cells. Once the cells are injected, they are allowed to settle to an empirically determined level of packing. This controlled centrifugation ensures the cells are optimally packed to receive the vector or reagent being delivered from the reagent septa 1116 and liquid transfer port 1170. When the valve is configured to allow so, that allows for precise introduction of reagents directly to the centrifugally concentrated cell population located at the bottom of the sedimentation column.

In one example, if the bottommost sensor detects that cells are being packed too densely, this information could be sent to the platform which then reduces the RPM of the centrifuge. The emitters may be activated in sequential or parallel configurations, depending on system requirements. For sequential activation, emitters may be enabled and disabled in a controlled, top-to-bottom manner along the vertical array of emitter/detector pairs. The delay between enabling sequential emitters is configurable, allowing precise adjustment to optimize detection sensitivity, resolution, or the dynamic properties of the cell suspension. A configurable delay may also be introduced before rerunning the sequence.

In use, eventually all the cells pass by the top emitter-sensor pair, and then after some amount of time based on particle density, fluid dynamics, and centrifugal forces, they will pass the middle emitter-sensor pair, and finally the bottom emitter-sensor pair. In practice, the system may slow or stop centrifugation after they pass the top pair of optical emitter-sensors, because once the cells have passed this level above the bottom of the sedimentation column, they are protected from being removed from the CPC during the liquid removal process, which is preferably performed via the application of positive pressure. The system is scalable to accommodate additional emitter/detector pairs, enabling flexibility for varying sizes of cassettes or similar apparatus. The number of emitter/detector pairs may be proportional to the size of the sedimentation column used for cell detection and sedimentation. The system may also include multiple arrays of emitter/detector pairs positioned at 90-degree offsets to allow for detection from two orthogonal angles, improving measurement accuracy and reliability.

In embodiments where the entire main chamber 424 or sedimentation column are not constructed of transparent or semi-transparent materials, at least one optically clear window in the sedimentation column 431 allows the sensor-emitter pairs 1062 to operate therethrough.

Real-time total cell concentration calculations are achieved by combining optical sensor data with the known cell suspension volume. The total volume of the suspension is identified either by weight (via strain gauges) or vertical height measurements (using laser or radar systems). The sedimentation column's narrow geometry significantly improves measurement sensitivity, facilitating detection of subtle changes in light transmission that correlate with the presence of target cells, such as hematopoietic stem and progenitor cells, T-cells, or NK cells.

To mitigate the limitation that the optical sensors only detect sediment within the sedimentation column, the system employs a swift but gentle mixing process immediately before measurement. This ensures cells are evenly distributed throughout the cell suspension volume. Once mixing is complete, the system tracks the elapsed time as the CPC rotates to a vertical, motionless position. This timing factor is critical for accurate algorithmic calculations of cell counts.

The system uses an empirically determined algorithm that may account for one or more of the following to refine cell count estimations, ensuring high accuracy even as sedimentation begins shortly after mixing ceases, the determination considering the elapsed time since mixing ceased; the light signal occlusion measured at each of the three optical sensor pairs; and sedimentation rates, which are essentially uniform across the sedimentation column and the bulk cell suspension above it.

The tapered geometry of the main chamber 424, which narrows from a wider top to a narrower bottom, ensures controlled movement of cells in suspension during cassette tilting. This motion, guided by gravity and the internal shape, can create uniform laminar flow mixing optimized for different solution volumes by adjusting rotation rates while minimizing sheer stress on cells. The tapered geometry, which narrows from a wider top to a narrower bottom, also ensures controlled movement of cells in suspension when the cassette rotates on its axis. This motion, guided by gravity and the internal shape, can create uniform laminar flow mixing optimized for different solution volumes by adjusting rotation rates.

To optimize performance, the interior walls of the CPC are constructed using a low-adhesion material, such as a biocompatible polymer with a smooth, diamond-polished finish. This surface treatment minimizes the likelihood of cell adhesion, ensuring that cells remain suspended in the fluid and can be effectively concentrated, sequestered, or removed as required. The low-adhesion material significantly reduces cell loss caused by sticking, thereby enhancing recovery rates and improving overall process efficiency. By carefully selecting materials and finishes that prevent adhesion, the CPC maximizes the yield of viable cells at every stage of the processing cycle, supporting consistent and high-quality results.

Effective and thorough mixing is essential for the cell manufacturing process. The provided geometry enables the CPC to perform essential steps such as cell washing, activation, gene modification, and harvesting within a closed, sterile, and automated environment. During these processes, cells must remain evenly distributed to ensure reliable cell selection, activation, and transduction. Uniform mixing also guarantees that all cells have equal interaction with reagents and vectors while maintaining a representative distribution for quality control sampling. Without this optimized mixing, cells would settle unevenly, leading to inconsistent recoveries, reduced activation efficiency, and lower transduction rates.

The natural tendency of cells to sediment and concentrate is overcome through the combination of tapered or conical geometry, precision rotation, and optimized fluid motion. During centrifugation or sedimentation processes, the tapered shape naturally directs sediment-such as target cells, including hematopoietic stem and progenitor cells, T-cells, or NK cells toward the narrow bottom section, referred to as the sedimentation column.

By ensuring swift and thorough mixing, the CPC's design prevents premature cell settling and maximizes exposure to reagents and genetic materials, significantly improving overall processing outcomes.

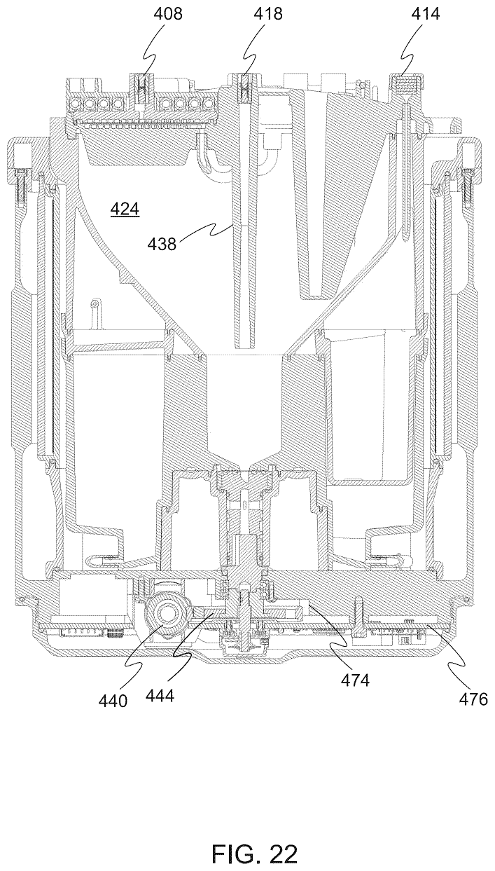

FIG. 22 depicts an alternative embodiment of the invention wherein a relative location of a worm gear-driven valve assembly is shown. The worm gear-driven valve actuator includes motor 440 which is positioned upon and electrically coupled to a printed circuit board assembly 476. While details of individual circuit components are not provided herein it should be noted that one of ordinary skill in the art would appreciate how the components on printed circuit board assembly 476 may be configured to provide power and control commands for operation of motor 440 when actuation of the valve assembly is required. Alternative embodiment FIG. 22 also depicts second gear 444 positioned within a recess defined by a downward-facing surface of bucket 120 that is shaped to receive second gear 444. First valve segment 454 is shown extending into an opening defined by lower housing component 406 of CPC 212.

Hydrophilic filter 422 is shown directly beneath fluid port 408 and is responsible for screening out any impurities in fluid being introduced through fluid port 408. Septa 414 and 416 have a tapered geometry to guide received syringe needles.

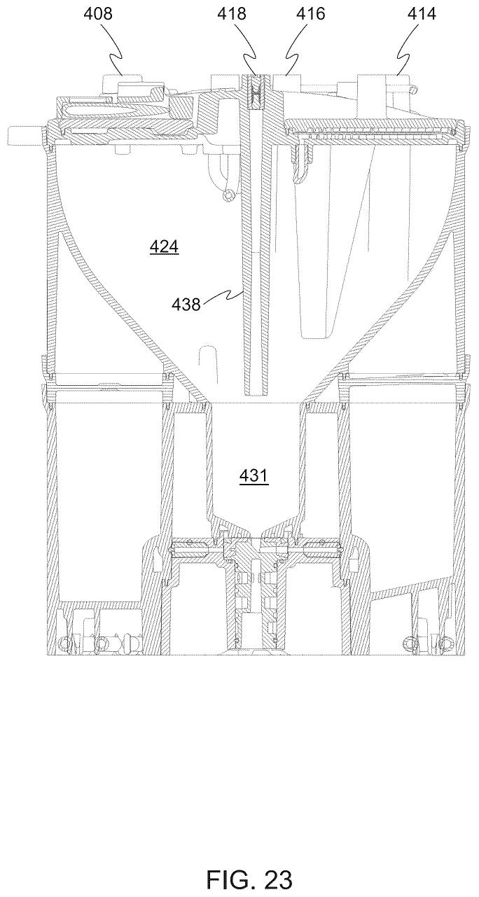

FIG. 23 shows a cross-sectional side view of CPC 212 taken along section line C-C and in accordance with a preferred embodiment of the invention.

As shown in FIG. 24, center of gravity (CG) of the bucket 120 varies based on liquid level. The pivot axis 1092 mixes cell suspension in CPC 212. FIG. 24 shows the changes in the CG at 350 ml as indicated at 1094 and 25 ml as indicated at 1096. The vertical, central positioning of the CG for the centrifuge bucket and the CPC 212 is critical for ensuring efficient operation during both mixing and centrifugation processes. Mixing of the cell suspension occurs only when the centrifuge bucket is not spinning centrifugally around the rotor. To minimize the torque required by the mixing motor, the pivot axis of the bucket/CPC assembly must be positioned very close to its CG. This ensures efficient mixing whether the cell suspension volume is as low as 15 mL or as high as 350 mL. By aligning the pivot axis near the CG, the system achieves smooth, controlled oscillation with minimal energy expenditure. When the bucket and CPC are rotating around the centrifuge rotor, the bucket rotates upward, approaching a 90° tilt. This occurs because the geometric center of the mass aligns closely with that of the counterbalance, ensuring near-perfect balance. This alignment provides the most uniform sedimentation rates for the cells, which move consistently away from the axis of rotation under centrifugal forces. If a perfect 90° rotation of the bucket/CPC assembly is required during centrifugal operation, the mixing motor can make precise adjustments due to the minimal torque demands in this state. Such fine control ensures optimized conditions for cell sedimentation and subsequent processing.

In one embodiment, The system uses a Relative Centrifugal Force (RCF) compensation mechanism designed to regulate the sedimentation rates of cells within the cell suspension as they enter the sedimentation column during centrifugation. If the sedimentation rate deviates from the empirically determined optimal rate—either being too fast or too slow—the RCF is dynamically adjusted to achieve ideal packing of cells at the bottom of the sedimentation column. This mechanism is particularly critical during spinoculation, where cells are co-mingled with vectors, to ensure efficient and uniform interaction between cells and vectors.

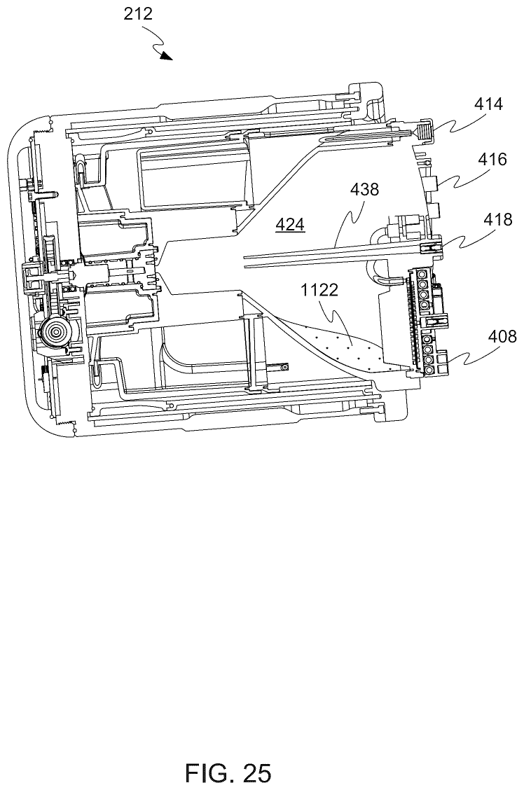

As shown in FIG. 25, for small volumes such as 100 ml the bucket 120 can rotate as much as 135 degrees in either direction for maximum mixing without liquid entering the fluid passage ports of the plurality of filters. The degree of possible tilt is thus inversely proportional to the amount of liquid in the main chamber. For instance, with 115 ml volume it may be able to tilt to about 120 degrees in either direction, or with 160 ml it may only be able to rotate to 110 degrees in either direction. The maximum capacity of liquid the CPC 212 can hold is 350 ml and the least capacity is 25 ml. The motor can rotate the bucket 120 to within one tenth of a degree of rotational accuracy. Thus, it is clear that the gravitational force is not the only cause for the rotation of the bucket 120. The pivot point of the bucket 120 is designed to maximize bucket 120 clearance during rotation.

FIG. 26 shows a view along cutline D-D of FIG. 20, looking upwards from inside the CPC to the underside of the filters 422, 426 and 428 of CPC 212. Filters 426 and 428 are preferably hydrophobic filters that allow sterile gas entry and exit, with structural integrity confirmed through automated pressure/decay testing, as well as hydrophilic filter 422 positioned under coiled tubing on the CPC lid, this filter allows sterile fluid transfer into the CPC, also verified by pressure/decay testing. Preferably three 0.2-micron pore size filters are integrated into the top of the CPC lid. These filters facilitate the aseptic transfer of fluids into the CPC and the controlled exchange of gases (entry and exit) while maintaining a sterile environment. The filters ensure that all fluid and gas pathways remain uncontaminated during cell processing.

FIG. 27 shows a view along cutline E-E of FIG. 20, looking down into the main chamber 424 and the waste standpipe 438, which provides a path by which waste solution within CPC may be evacuated through waste port 418. Also shown is a plurality of ribs designed to improve thermal transmission from the heater in the bucket 120 to the fluid in the CPC 212.

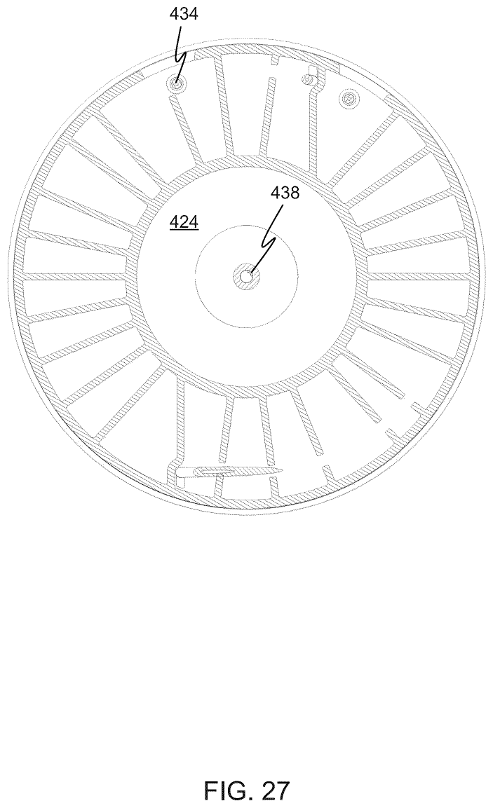

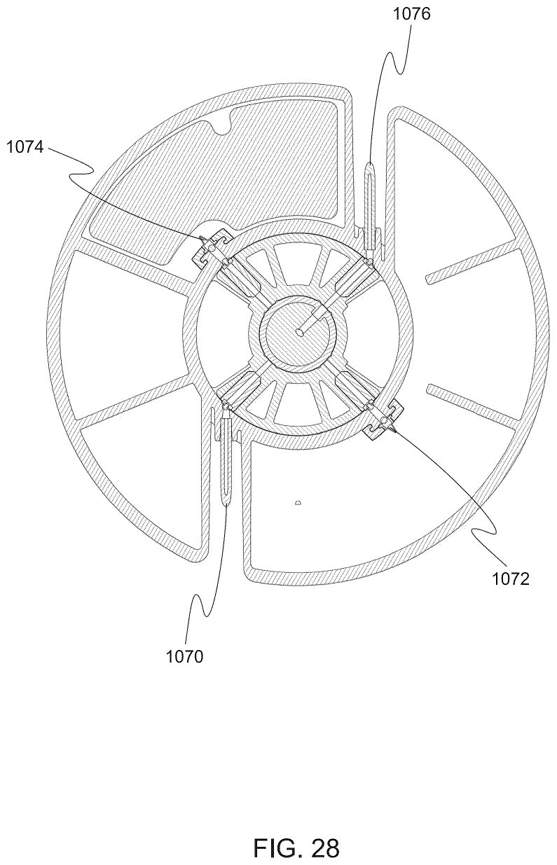

FIG. 28 shows a view along cutline F-F of FIG. 20, showing the four rotational positions of the valves. The valve rotates to four positions, each creating a different fluid connection for a different process. Positioning between these four positions create no fluid connections and here the CPC valve is said to be parked or closed. When the valve is at position 1070, a fluid connection is open allowing the CPC to receive reagents from a reagent septum, position 1072 to create a fluid connection for the sequestration of non-target cells, location 1074 creating a fluid connection with a compartment for the sequestration of 2^{nd} fraction T-cells or non-target cells preferably into a 30 mL volume limited compartment, and location 1076 which creates a fluid connection out of the CPC chamber to a harvest septum and ultimately to a harvest vessel. When the valve is configured to allow so, reagent septa 1116 connects to valve position 1070, a liquid transfer port, with a conduit tube that allows for precise introduction of reagents directly to the centrifugally concentrated cell population located at the bottom of the sedimentation column 431. This targeted delivery maximizes reagent efficiency and enhances cell processing outcomes.

The top of the CPC in the ACP is equipped with multiple input and output ports, each playing a critical role in maintaining a sterile and controlled environment for CAR T-cell manufacturing. These ports enable the efficient, aseptic transfer of fluids, gases, and samples through the lid, supporting the entire cell processing workflow—from the initial introduction of cells to the final harvest of gene-modified cells. Each input/output septa port is integrated with a UV mechanism that disinfects opposing septa during docking. There is a septum for each syringe located on the bottom of the TSC which will be one of the two opposing septa to be disinfected by the UV disinfection module during any transfer of fluids. The opposing septa that will be simultaneously disinfected will be on either the RSC containers or the lid of the CPC. Specifically, the UV mechanism ensures that both the TSC septa and the CPC lid septa, or the RSC septa, are disinfected during reagent retrieval or sample deposition. Also, Hydrophobic filters allow sterile gas exchange, while hydrophilic filters manage the aseptic transfer of fluids. Both filter types undergo pressure/decay testing to confirm their structural integrity before and after processing clinical populations of gene-modified cells.

The aseptic transfer ports on the CPC lid preferably include both septa ports and filter ports. Input and output septa are positioned on the CPC lid to align precisely with the TSC syringes, enabling aseptic transfer of reagents, samples, and harvested cells.

This strategic combination of port design, UV disinfection, and filtration systems ensures aseptic entry and exit of gases and fluids, effectively preventing contamination and preserving the purity of the CPC's internal environment. By maintaining these sterile conditions, the ACP enables the precise and automated execution of complex cell processing tasks, ensuring high-purity CAR T-cell production with minimal risk of contamination.

The CPC 212 is designed with dedicated input and output ports, ensuring sterile and efficient handling of gases, fluids, reagents, and samples throughout the cell processing cycle. Beginning first with the input ports, these may be categorized into four groupings. (1) Gases-in: Hydrophobic filters on the CPC lid allow sterile gas entry without contaminating the interior environment. These filters also enable aseptic release of displaced air when fluids are introduced into the CPC, maintaining cell culture conditions. (2) Fluids-in: Fluids required for cell processing, such as washing solutions, culture media, or transduction agents, pass through hydrophilic filters. The filters have a larger surface area to accommodate the higher fluid flow rates necessary for efficient processing. (3) Reagents-in: A dedicated input port provides controlled access for introducing specific reagents, such as those for cell selection, activation, or genetic modification. (4) Sealed Tubing with Clot Filter: Tubing connected to blood or leukapheresis bags enables sterile docking and entry of the patient's cells. The tubing includes a clot filter to remove unwanted clots before cells enter the CPC. After transfer, the tubing is sealed one inch from the CPC lid entrance and secured in a nearby plastic clip to ensure safety and avoid obstruction.

Turning now to the output ports, these may also be categorized into four groupings. (1) Gases out: A hydrophobic filter ensures that gas leaving the CPC is sterile and free of contaminants. (2) Harvested Gene-Modified Cells-out: dedicated septum allows for the aseptic final harvest of the gene-modified cell product after processing steps are complete. (3) Waste Fluids-out: This port, equipped with a one-way valve, safely expels waste fluids after cells are centrifugally sequestered at the bottom of the sedimentation column. Optical sensors confirm cell positioning before waste removal, and positive air pressure propels the waste fluid into an expandable, sealed container at a controlled rate, preventing the unintended removal of sequestered cells. (4) Samples-out: A septum port allows the TSC to aseptically collect cell suspension samples at various stages of the process for quality control or monitoring, without disrupting the sterile internal environment.

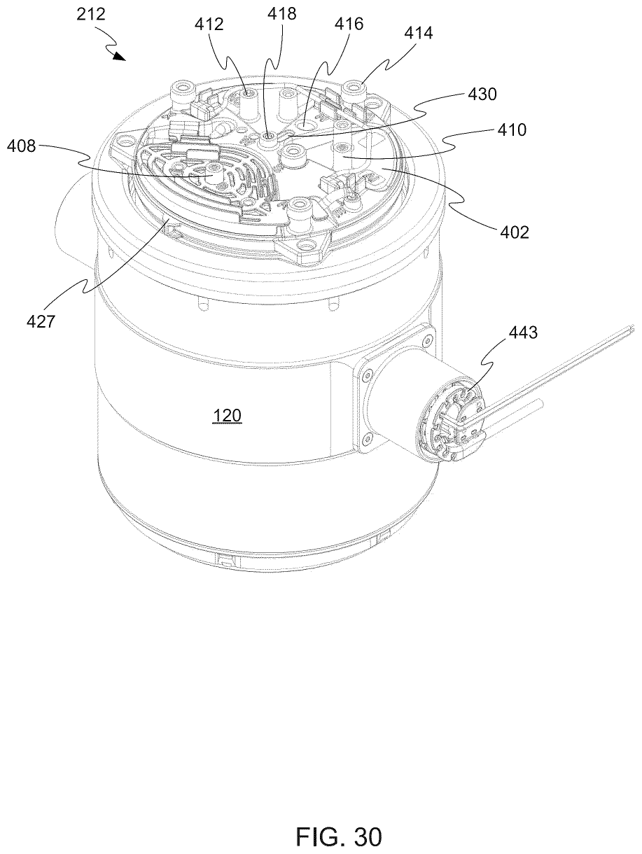

FIG. 30 show the top perspective view of the CPC 212. The septums 414 and 416, are configured to interact with and receive fluid supplied by injectors of TSC 210 or allow for collection of fluid by extractors of TSC 210. Specifically, the septums include harvest septum 414 and dissolved O2 well septum 416.

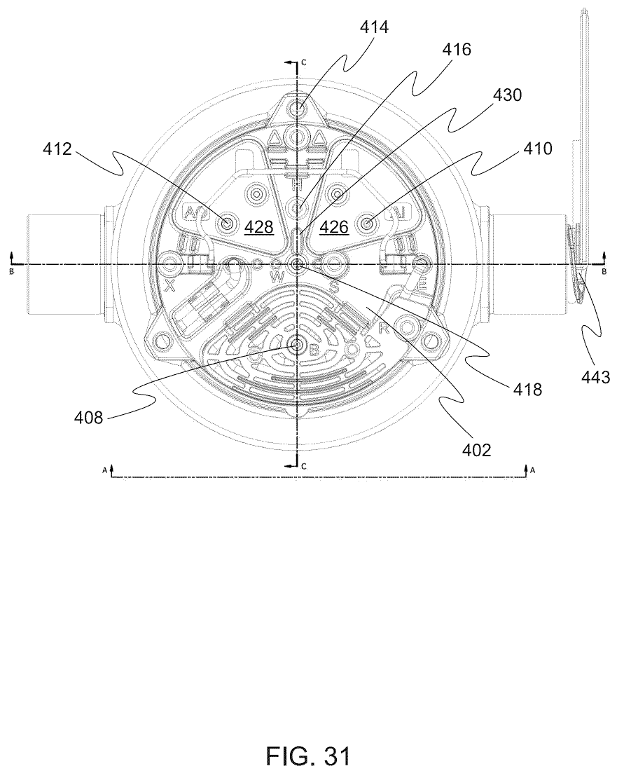

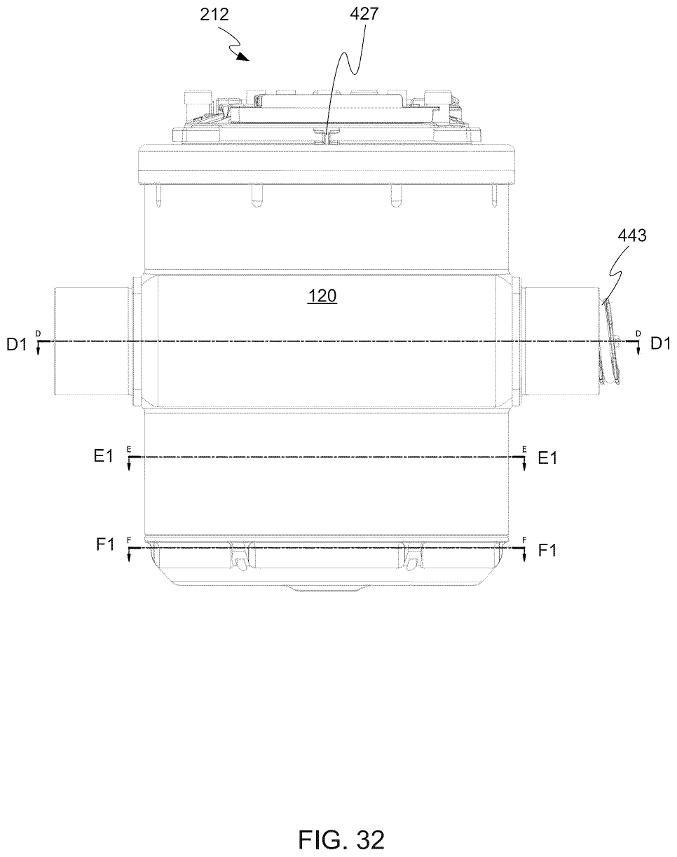

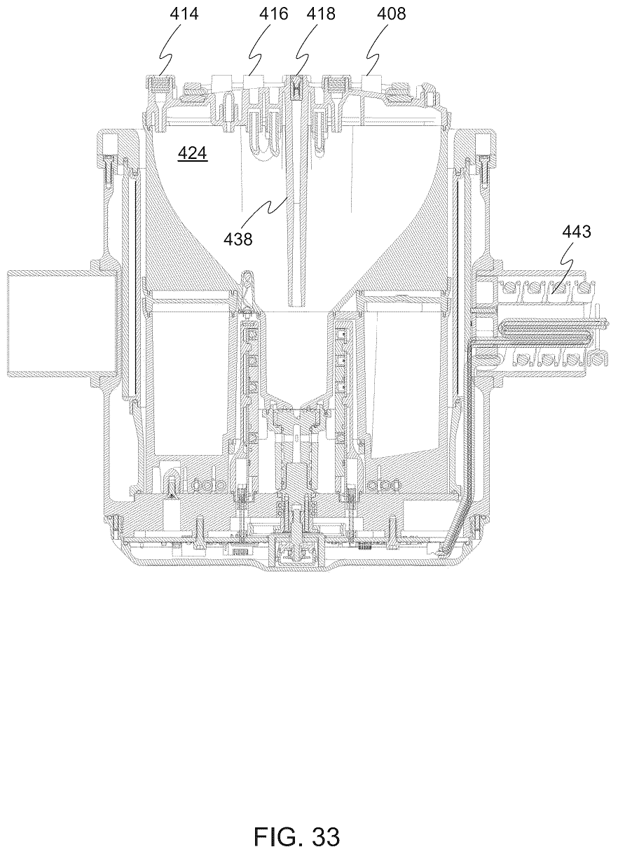

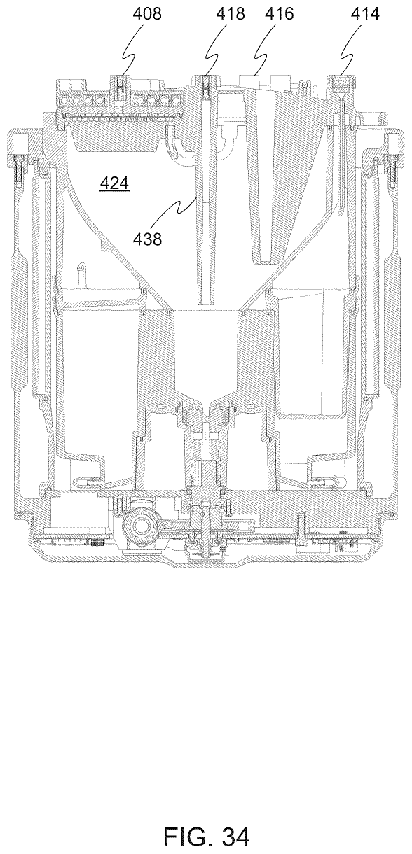

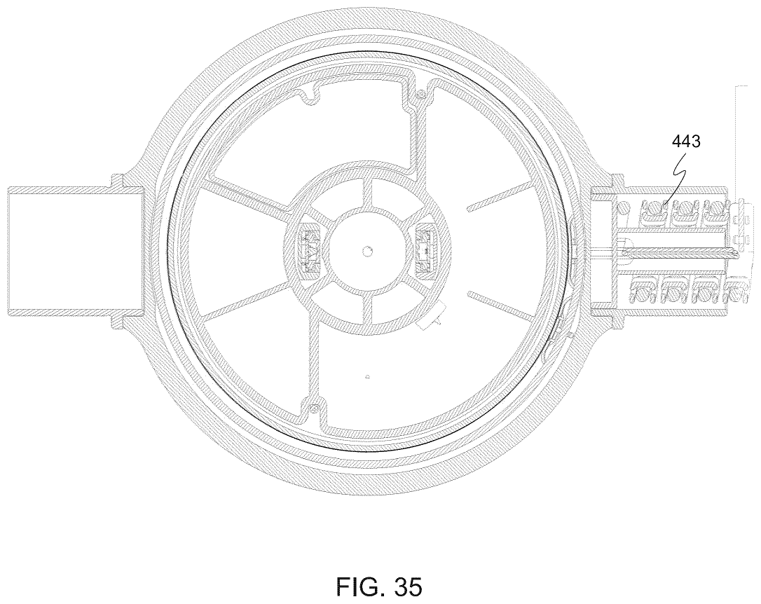

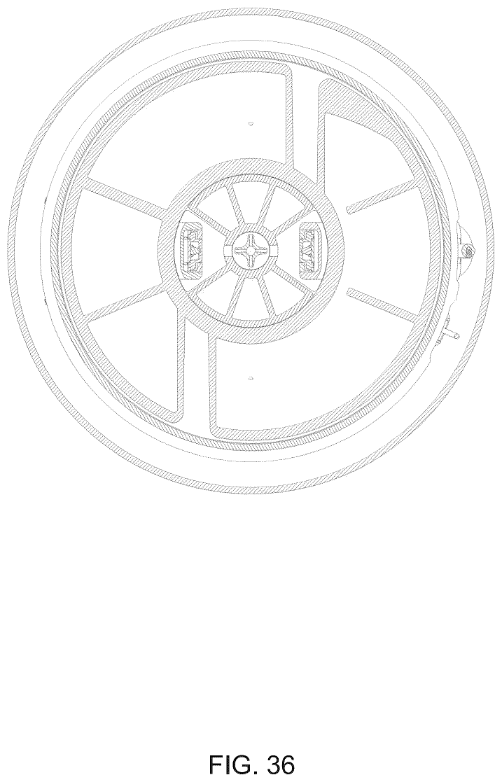

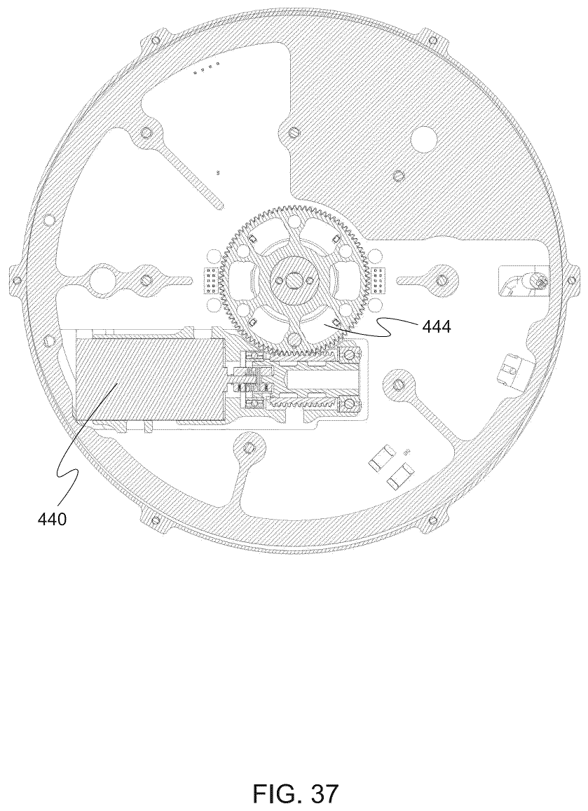

FIG. 31 also shows side view lines A-A, and section lines B-B and C-C, which define the views provided in FIGS. 32, 33 and 34, respectively. FIGS. 33-34 are cross sectional views while FIG. 32 is a side view with section lines D1-D1, E1-E1 and F1-F1, which are depicted as FIGS. 35, 36, and 37, respectively.

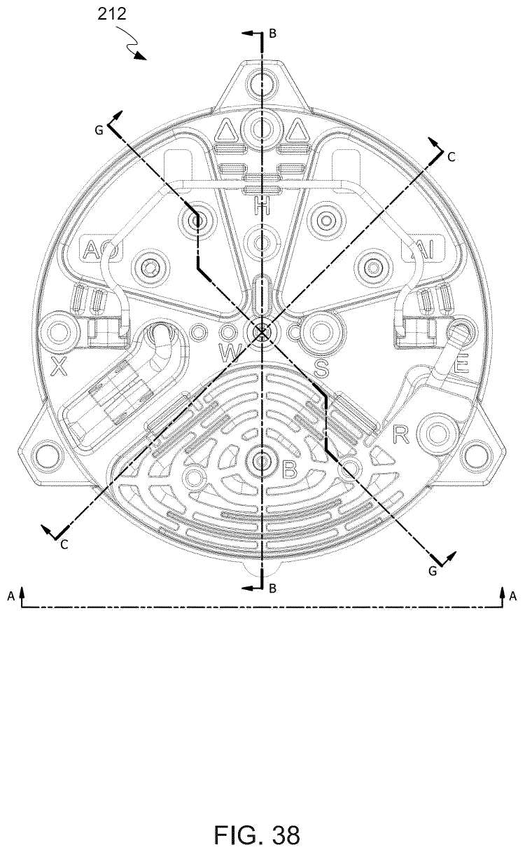

FIG. 38 shows the top of the CPC 212. The figure also shows view line A-A and section lines B-B, C-C, and G-G, which define the views provided in FIGS. 20, 21, and 59B-59D. View A-A is shown in FIG. 20, Section line B-B extends through fluid port 408, waste port 418 and septum 414 and is shown as FIG. 21.

The pneumatic system within the ACP leverages precise gas pressure control to execute multiple critical functions throughout the cell processing workflow. First, it conducts pressure decay tests to verify the integrity of hydrophobic and hydrophilic filters within the CPC 212, ensuring the sterility and reliability of fluid and gas transfers. Second, it utilizes pressurized gas to implode microbubbles, facilitating cell release during certain processing steps. Third, the system applies pressure to the top of the PFC, enabling fluid transfer through a pie-shaped filter exit receptacle into the CPC chamber. During this process, radar or laser sensors monitor the fluid volume entering the chamber to ensure accuracy. Fourth, the pneumatic system pressurizes waste liquid lines to expel waste fluids out of the CPC while simultaneously preventing contamination. Once the liquid is removed, gas is applied to clear any residual liquid from the lines. Lastly, the pneumatic system propels genetically modified cells into the harvest syringe, ensuring precise and aseptic transfer of the final cell product for collection. These versatile capabilities enhance the ACP's efficiency and reliability in delivering high-quality cell therapies.

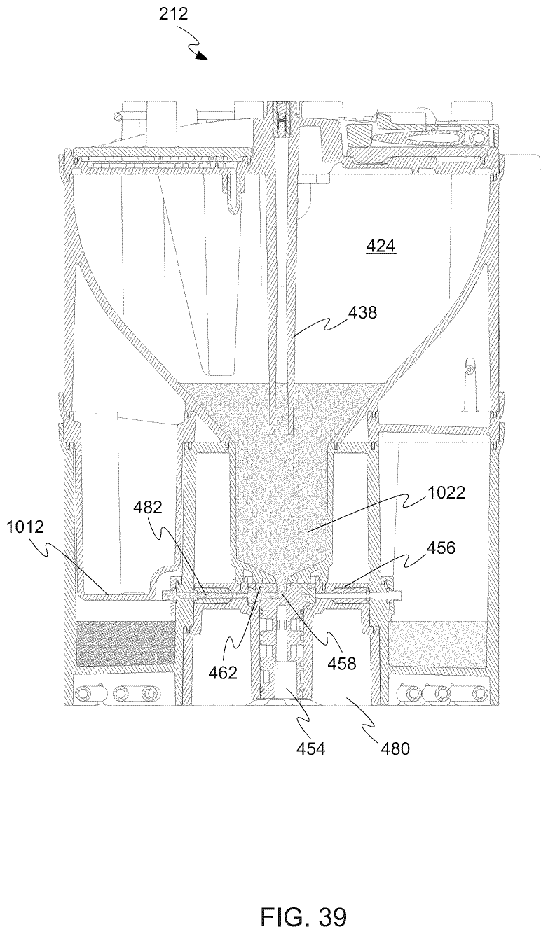

FIG. 39 is a cross-sectional view showing CPC 212 undergoing a centrifugation operation in which target cell solution 1022 is pressed toward the bottom of main chamber 424 and then first valve segment 454 is rotated to align first channel segment 458 with second channel segment 482, as depicted.

A port enables the aseptic passage of the gene-modified and formulated target cell solution. The thin tube connects to a TSC syringe, which completes the transfer of the cell solution to the RSC final harvest container with minimal to no loss of cells. This allows target cell solution 1022 to be collected in target cell primary fraction chamber 480. Once a desired amount of target cell solution 1022 is added to target cell primary fraction chamber 480 additional target cell solution 1022 is added to target cell secondary fraction chamber 488 (see FIG. 22) by rotating first valve segment 454 such that first channel segment 458 aligns with a channel segment associated with target cell secondary fraction chamber 488.

The centrifugation operation may be terminated once the primary and secondary fraction chambers 480, 488 have received target cell solution 1022. Prior to removal of processing cassette 212 from cell processing device 100 and retrieval of the final therapeutic dose or doses, cell processing device 100 may report on any irregularities noted during the process as evidenced by the sensor readings monitored throughout the cell processing operation. Filters 422, 426 and 428 of processing cassette 212 may also be checked for integrity and any irregularities with the filters can also be reported. The pre-processing and post-processing filter integrity may be verified using a pressure decay method to individually check each filter. Pre-processing and post-processing filter integrity checks may be performed within cell processing device 100 by a testing cassette positioned within cassette positioning assembly 132. The testing cassette may be configured to apply a known gas pressure to the inlet port of each filter (422, 426 or 428) while blocking all the exit ports. Once pressurized, a sterile venting port (i.e. 412 or equivalent) is opened allowing gas flow only through the filter being tested. Measuring the pressure decay time in the inlet port volume against known limits determines the integrity of the filter. Filter integrity testing results are incorporated in the lot release report.

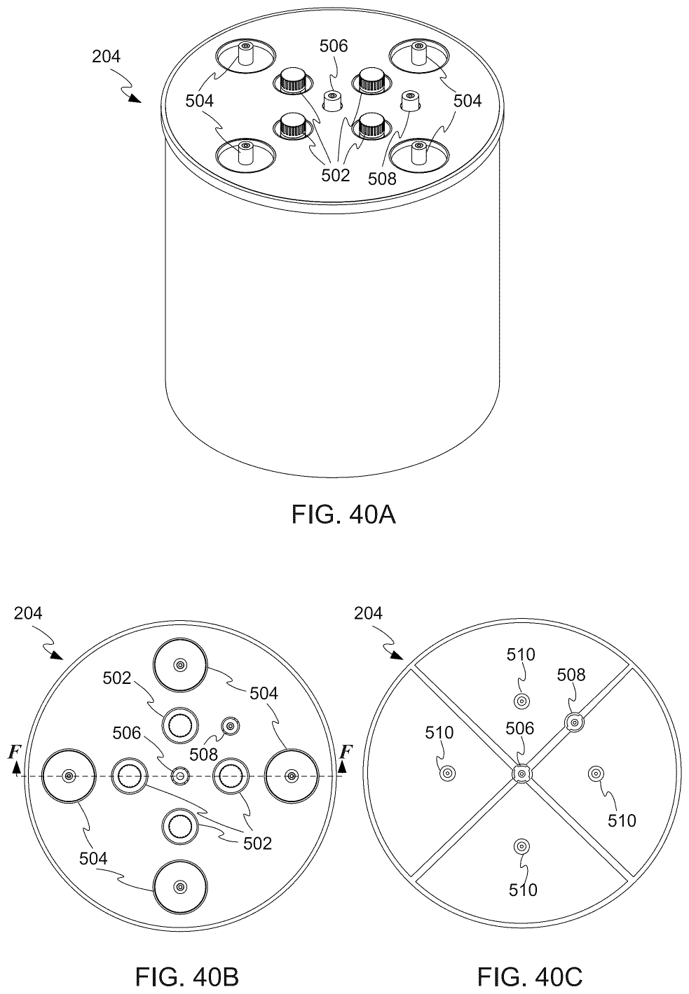

FIGS. 40A-40C show various views of PFC 204. FIG. 40A shows a perspective view of PFC 204 and how it has an overall cylindrical geometry. An upper surface of PFC 204 includes multiple needleless entry ports 502 for adding solution into PFC 204. Addition of solution into PFC 204 is typically performed prior to placing PFC 204 into cell processing device 100. While four needleless entry ports 502 are depicted and correspond to four pie shaped chambers within PFC 204, it should be appreciated that PFC 204 could be divided up into a larger or smaller number of pie-shaped chambers. PFC 204 can alternatively include only a single chamber holding a single type of fluid in which case cassette positioning assembly 132 could include multiple fluid cassettes with one fluid cassette for each type of liquid needed. Each chamber of PFC 204 further comprises a corresponding pneumatic port 504 configured to receive a fixed amount of compressed air for ejecting precise amounts of fluid from a corresponding chamber of PFC 204.

Pneumatic ports 504 may each include a filter for preventing the unintentional addition of contaminates to PFC 204 when pressurizing a particular chamber of PFC 204. The PFC lid preferably contains four filters, one for each of its four pie-shaped compartments. Each compartment can hold up to 500 mL of buffer or other fluids. The 0.2-micron filters allow aseptic passage of air under controlled pressure (1 to 5 psi), which regulates the flow rate of fluids through the docking port and the hydrophilic filter on the CPC lid.

In some embodiments, an amount of fluid ejected by PFC 204 into processing cassette 212 can also be controlled based on feedback from the radar or laser level sensor. FIG. 40A also shows an upper end of a waste pass through 506 that extends along a longitudinal axis of PFC 204 that allows waste sample material exiting waste port 418 of processing cassette 212 to continue upward and into waste tube 208 and into a flask for collection. An upper end of pneumatic pass through 508 is also shown. Pneumatic pass through 508 allows for air to be added to or removed from processing cassette 212 by applying positive or negative pressure to pneumatic pass through 508 while pneumatic pass through 508 is coupled to a pneumatic port of processing cassette 212. Any applied air pressure in each pie-shaped compartment is carefully managed to ensure precise fluid transfer into the CPC, supporting efficient and sterile processing.

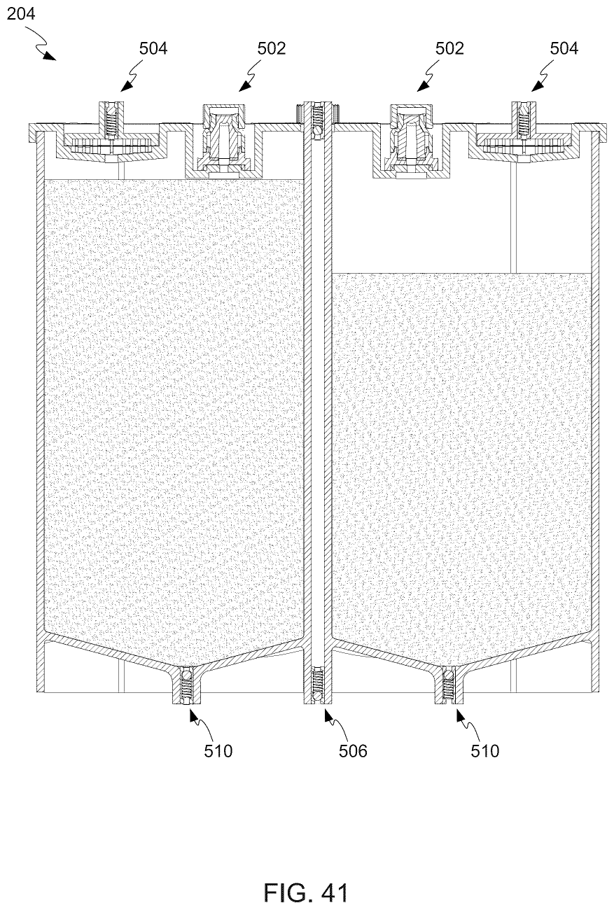

FIG. 40B shows a top view of PFC 204 showing how pneumatic ports 504 are positioned along a periphery of PFC 204 and needleless entry ports 502 are arranged in a central region of PFC 204. Waste pass through 506 is located at the center of PFC 204 and pneumatic pass through 508 is shown outboard of needleless entry ports 502 and inboard of pneumatic ports 504. FIG. 40B also shows section line F-F, which corresponds to FIG. 41.

FIG. 40C shows a bottom view of PFC 204 that includes another end of waste pass through 506. The opposing ends of waste pass through 506 are generally connected by a flexible or rigid tube that extends along a longitudinal axis of PFC 204. FIG. 40C also shows another end of pneumatic pass through 508, which is configured to engage pneumatic ports 410 and 412 on processing cassette 212. Fluid exit ports 510 are also shown and are configured to engage fluid port 408 on processing cassette 212.

FIG. 41 shows a cross-sectional side view of PFC 204 in accordance with section line F-F from FIG. 40B. In particular, FIG. 41 shows two filled fluid chambers of PFC 204 as well as internal components of needleless entry ports 502, pneumatic ports 504, waste pass through 506 and fluid exit ports 510. In particular, waste pass through 506 and fluid exit ports 510 include check valves to prevent contamination of the fluid stored within PFC 204 when PFC 204 is not in use. For example, the check valve of waste pass through 506 is configured to compress the spring when pressurized sample material exits processing cassette 212 and into waste pass through 506.

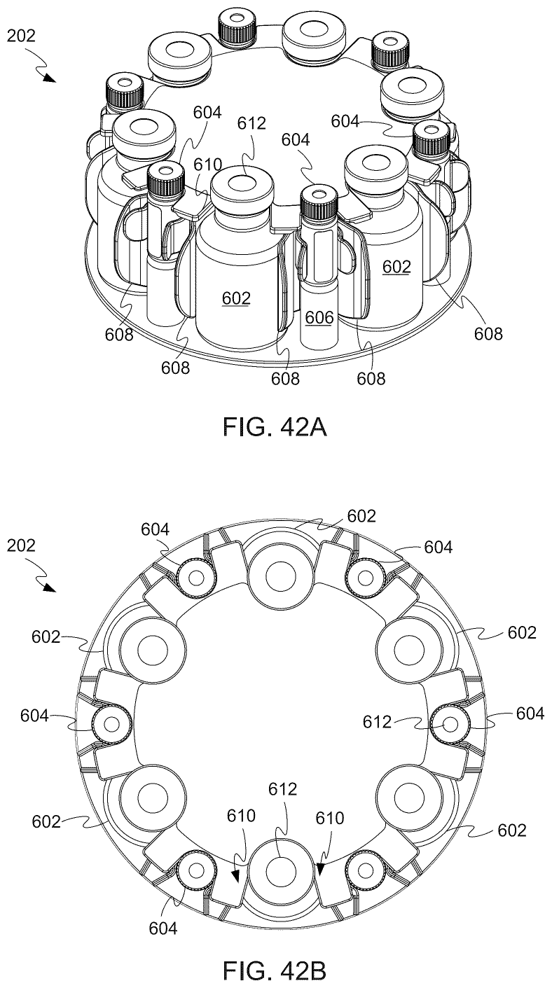

FIGS. 42A-42B show different views of RSC 202. FIG. 42A shows a perspective view of RSC 202 and FIG. 42B shows a top view of RSC 202. A total of six reagent vials 602 and six prelabeled sample vials 604 are depicted in FIGS. 42A-42B, however, it should be appreciated that a smaller or larger number of vials is possible and considered to be within the scope of the invention. The RSC 202 houses up to six pre-filled reagent vials that will be used through the cell processing, preferably each with a volume capacity of up to 20 mL. These vials may contain essential components such as microbubbles, vectors, and linkers. Additionally, the RSC 202 contains designated sample vials, ready to receive cell samples for quality control analysis throughout the processing cycle. Sample vials 604 are positioned on stands 606 so that upper surfaces of sample vials 604 are flush with upper surfaces of reagent vials 602. Each of the vials may be slid into place on RSC 202 by sliding the respective vial into clips 608, which in combination with collars 610, prevents lateral and vertical movement of the vials while installed on RSC 202. Each of vials 602 and 604 include a cap with a region 612 configured to be pierced by a needle that allows for insertion or extraction of fluids from the vial. The system may include an automated quality control in the form of integrated sensors monitoring syringe volume and flow rates to detect any deviations, triggering alerts and adjustments as necessary.

The ACP features a UV disinfection module specifically designed to disinfect port septa prior to each fluid transfer, thus each needle transfer occurs under aseptic conditions, as the Disinfection Module (DM) uses intense UV radiation or light to disinfect the surfaces of both the TSC septa and the opposing RSC or CPC septa. The UV light is focused on two opposing septa preceding each needle penetration of either septa. A needle will emerge from a bottom septa of the TSC and down into either a septa of the CPC lid or a septa of the RSC only after both septa have been disinfected. The UV disinfection module is mobile and will always be precisely located to assure aseptic transfers involving the TSC and the RSC or CPC. No needle will ever be extended outside of the TSC except when it is docked with the UV component and mechanically operated and under automated control by the system software. Preferably, a syringe will penetrate through a septum on the CPC lid and a septum on the sample vial 604, or a septum on the CPC lid and a septum on the reagent container. Septa may preferably be found on top of every sample vial 604 or reagent vial 602, the lid of the CPC, and the TSC itself has septa. Key components include:

UV-C Source: The module utilizes a UV-C wavelength light or radiation source (254-280 nm), which is optimal for microbial inactivation, effectively eliminating bacteria, viruses, and other contaminants on the septa surfaces. The UV light source is positioned to provide full exposure to the septa.

Reflective Interior Coating: The UV sterilization chamber utilizes UV reflective materials that directs the UV-C light, ensuring complete and uniform exposure to the septa from multiple angles. This design minimizes the risk of any shadowed or missed areas on the septa, achieving comprehensive disinfection before the syringe needle pierces any septa.

Automated Detection and Activation System: The module includes an optical sensor that detects the approach of the syringe toward the entry port. Once detected, the control system automatically initiates a disinfection cycle, activating the UV radiation source for a precise duration. This automated activation eliminates the need for manual operation, ensuring that each septa is sanitized just before use, reducing the possibility of contaminants settling on the septa post-disinfection.

Timing and Control System: The UV module is programmed to deliver a controlled, timed exposure (typically between 5 to 15 seconds) to achieve effective disinfection without degrading the septa material. Integrated with the ACP's central control system, the timing mechanism is calibrated to match processing needs and UV exposure requirements, allowing for customized cycles depending on usage needs and material specifications.

In use, the UV sterilization module follows a streamlined process for disinfecting septa as part of the automated fluid handling sequence: Syringe Detection: The optical sensor detects the approach of a syringe as it nears the CPC entry port, triggering the disinfection cycle. UV Exposure: The UV radiation source is activated to disinfect the surfaces of the opposed septa prior to the penetration of both septa by the TSC syringe needle and continues until the transfer has taken place and the TSC needle has withdrawn above its septa. The reflective interior material ensures even UV exposure, effectively inactivating microbial contaminants on all exposed surfaces within the UV disinfection module. Completion and Shut-Off: Following the disinfection cycle, the UV radiation source is deactivated.

The UV disinfection module is validated to achieve a preferably 6-log reduction in microbial load on the septa surfaces, ensuring aseptic conditions during fluid transfers. The sterile integrity of the TSC syringe needles, originally achieved through gamma radiation of the packaged cassette prior to use with the ACP, remains uncompromised during the UV sterilization process, meeting all industry standards for aseptic processing environments. The module delivers approximately 750 mJ/cm² of UV energy to achieve this high sterilization efficacy, utilizing 170 mW of continuous direct current over 4.4 seconds. While direct current is currently standard, future designs may incorporate pulsed electromagnetic energy to enhance efficiency further.

Sample vials 604 are generally empty at the beginning of an operation being performed by cell processing device 100 and are gradually filled with samples taken from processing cassette 212 over a course of the operation being performed. This allows lab technicians operating the apparatus to check the samples over the course of the operation to validate that the operation is proceeding as expected. Reagent vials 602 generally contain fluids at the start of the operation, which are extracted by TSC 210 and transferred to processing cassette 212 over the course of the operation. Exemplary fluids contained within reagent vials 602 include microbubbles, antibody linkers and disease vectors. In one embodiment each of reagent vials 602 carries up to 20 mL of fluid and each of sample vials 604 carries up to 5 ml of fluid.

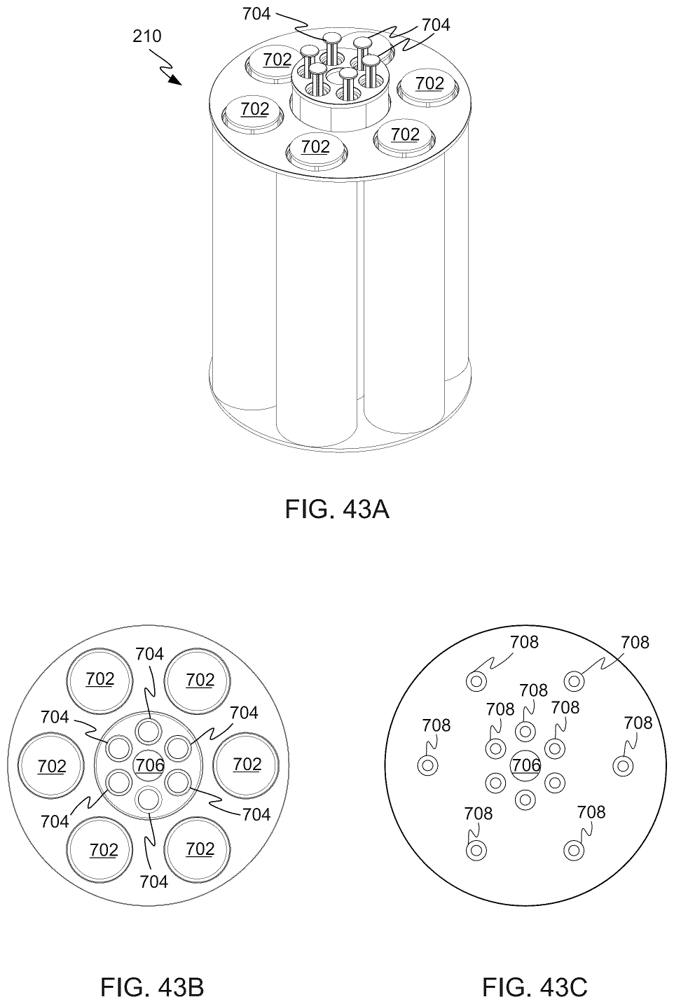

FIGS. 43A-43C show various views of TSC 210. FIG. 43A shows a perspective view of TSC 210 and how it can include multiple reagent syringes 702 arranged in a first circular configuration and multiple sampling syringes 704 arranged in a second circular configuration disposed within the first circular configuration. A number of reagent and sampling syringes 702 and 704 on PFC 204 will generally correspond to a number of reagent and sample vials 602 and 604 contained on RSC 202. While only a plunger portion of each of syringes 702 and 704 are visible in FIGS. 43A-43B, it should be appreciated that each of syringes 702 and 704 further comprises at least a needle and a solution receptacle.

FIGS. 43B-43C show respective top and bottom views of TSC 210, a key component of the TSC, designed to manage precise fluid transfers between other platform cassettes, preferably at least the CPC and RSC. The TSC houses multiple independently controlled syringes that transfer fluids, such as reagents, buffers, and cell suspensions, with a high degree of accuracy, maintaining sterility and consistency throughout various stages of cell processing. By integrating independent syringe control, pressure regulation, and a rotational docking mechanism, the TSC provides a reliable and effective means of managing fluids in closed-loop cell processing, ensuring consistency, safety, and scalability in cell therapy manufacturing and other biological applications.

FIG. 43B shows how in some embodiments the syringe cassette may include a waste pass through 706 for removing waste sampling materials from CPC 212. In the event waste pass through 706 was applied to TSC 210, it would also generally include a pneumatic pass through for supplying positive pressure to TSC 210 sufficient to drive waste sample material through waste pass through 706. FIG. 43C shows how each of syringes 702 and 704 can have a corresponding port 708 through which their needle extends through septa to add or subtract fluid from a main chamber of processing cassette 212 or from vials on RSC 202. A first subset of ports 708 are arranged along a periphery of PFC 204 correspond to reagent syringes 702 and a second subset of ports 708 arranged within a central portion of PFC 204 correspond to sampling syringes 704.

The TSC 210 manages the precise transfer of reagents and fluids into the CPC 212 thereby enabling accurate volumetric dosing for processes such as washing, activation, and transduction.

The TSC 210 is a key component of the ACP, designed to manage precise fluid transfers between other platform cassettes, such as the CPC 212, and RSC 202. The TSC 210 houses multiple independently controlled syringes that transfer fluids, such as reagents, buffers, and cell suspensions, with a high degree of accuracy thereby maintaining sterility and consistency throughout various stages of cell processing.

The structure of the TSC 210 may comprise a TSC housing, syringes, rotational docking mechanism, fluid transfer channels and ports, pressure control system and feedback sensors. The cassette housing is a structurally reinforced, closed housing containing a series of syringes arranged in parallel, each within a designated compartment. The housing provides sterile containment and is designed to prevent cross-contamination between fluids handled by different syringes. Each syringe within the TSC 210 is configured to hold specific fluid volumes, enabling independent control of fluid types such as culture media, buffer solutions, or processed cell suspensions. The syringes are equipped with adjustable plungers for volumetric accuracy.

The rotational docking interface enables alignment with other platform cassettes. This mechanism allows the TSC 210 to pivot between docking positions for each cassette, specifically the CPC 212 and RSC 202, ensuring precise fluid transfer across different stages of cell processing. TSC 210 is integrated with fluid transfer pathways and docking ports that align with corresponding ports on the CPC 212 and RSC 202. These channels are sterile and sealed with automatic check valves that open only when the TSC 210 is docked with a specific cassette thereby preventing contamination and ensuring fluid containment. Each syringe is equipped with a pressure control system that enables fluid dispensation at a range of pressures from approximately 1.5 to 45 PSI. This control is critical for managing various fluid types and viscosities, ensuring precise flow rates during dispensing into the CPC 212 or collection from the CPC 212 into the RSC 202. The TSC 210 includes feedback sensors positioned to monitor fluid levels within each syringe, providing real-time feedback on dispensed volumes and fluid levels. These sensors communicate with the ACP's central control system to adjust fluid volumes and pressures dynamically.

The TSC 210 aligns with the CPC 212 during stages requiring fluid transfer into or out of the cell processing environment. For instance, during washing and activation stages, the TSC 210 dispenses reagents, buffers, and activation solutions into the CPC 212 at precise volumes and pressures to achieve optimal cell washing, isolation, or activation. During cell modification stages, during gene insertion or modification stages, the TSC 210 transfers viral vectors or genetic reagents to the CPC 212, ensuring even distribution through controlled syringe actuation.

The TSC 210 can also dock with the PFC 204 to draw specific fluids required for cell processing. The PFC 204 serves as a reservoir for various buffers, media, and reagents, which the TSC 210 transfers to the CPC 212 as needed. The TSC 210 syringes draw precise amounts of fluids from the PFC 204 to inject into the CPC 212 for specific processing steps, including cell separation, incubation, or buffer exchanges.

The TSC interacts with the RSC. Regents may be obtained by the TSC 210 from the RSC and then transferred to the CPC212 via at least one syringe, wherein each large volume syringe in the TSC 210 selectively draws fluid from the RSC and injects it into the CPC 212 and each small syringe in the TSC 210 draws cell suspension samples from the CPC and injects them into a sample vial in the RSC 202 septa. After completing the cell processing steps, by aligning the TSC 210 with the CPC 212, the TSC 210 selectively retrieves gene modified and formulated cell suspensions from the CPC and dispenses them into the harvest container of the RSC for final storage or analysis. The TSC 210 transfers the final cell product, such as activated or genetically modified cells, from the CPC 212 to the RSC 202, ensuring sterile containment and minimal cell loss during transfer By aligning the TSC with the CPC, wherein the TSC selectively retrieves gene modified cell suspensions from the CPC and dispenses them into the RSC.

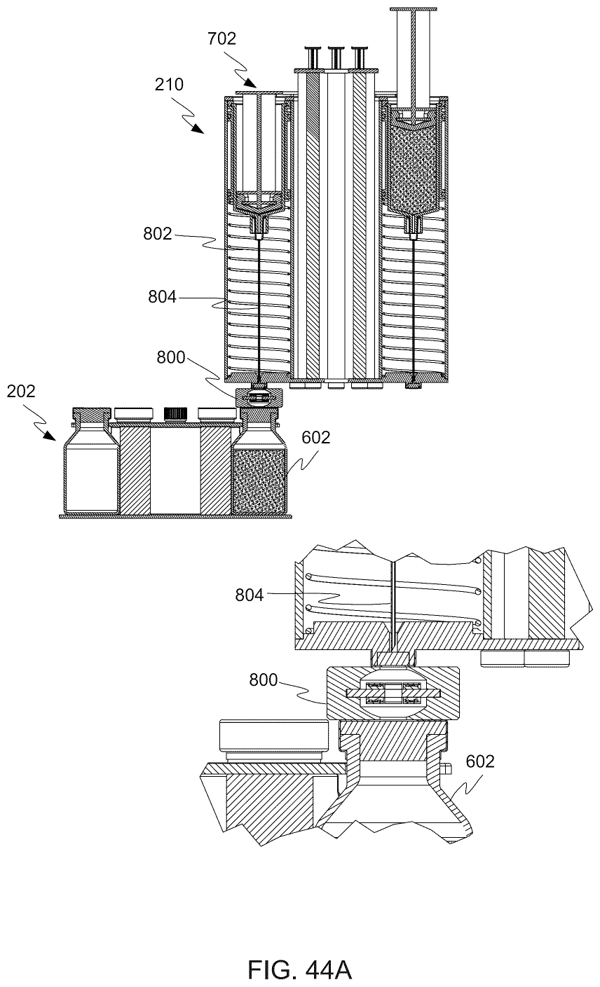

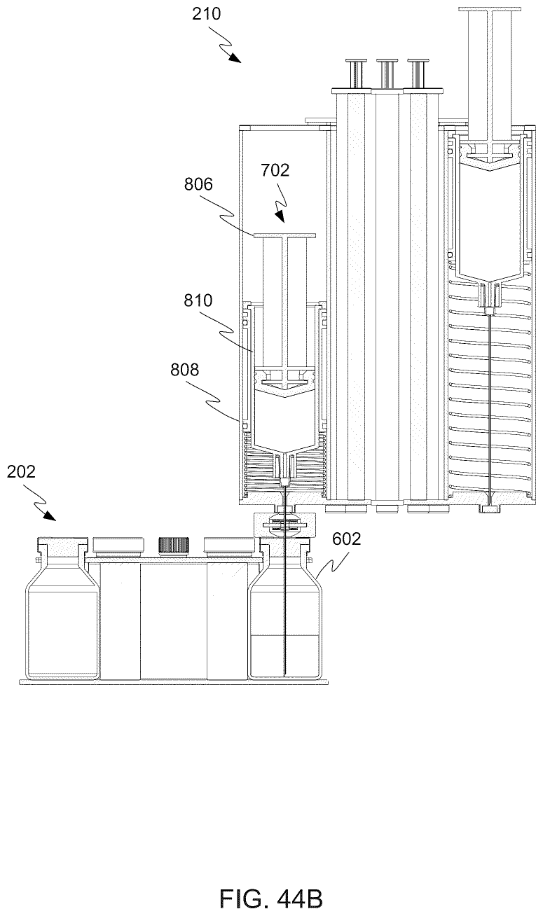

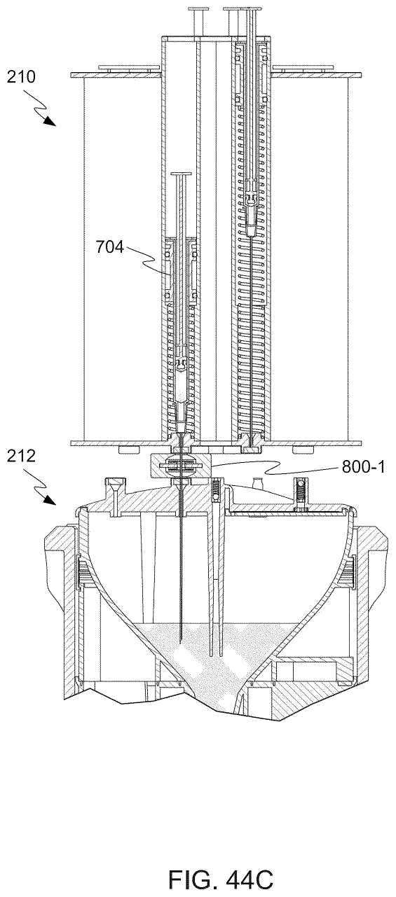

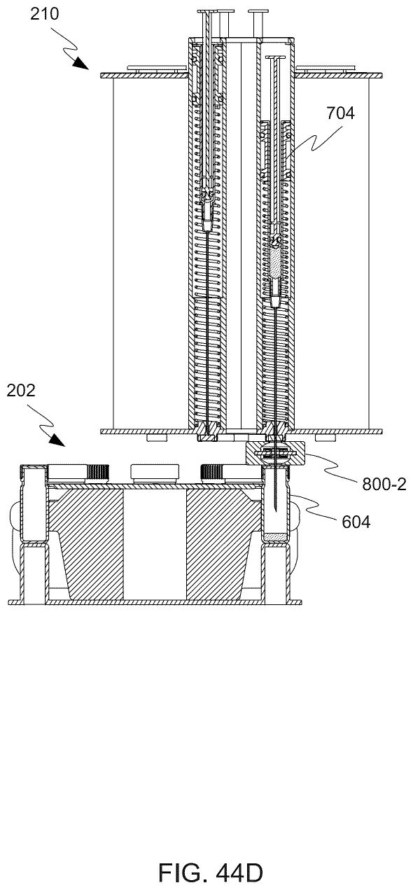

FIG. 44A shows a cross-sectional side view of TSC 210 interacting with RSC 202. In particular, TSC 210 is shown engaging RSC 202 such that syringe 702 is aligned with vial 602. Syringe 702 is held in position at the top end of TSC 210 by spring 802. A UV sanitizer 800 is positioned between RSC 202 and TSC 210, which is configured to sanitize exterior surfaces of the septa of the TSC 210 and RSC 202 in order to prevent contamination of a needle of syringe 702. While UV sanitizer 800 is not shown attached to another device, it should be appreciated that UV sanitizer 800 may be positioned between RSC 202 and TSC 210 by a swing arm capable of precise positioning of UV sanitizer 800. Since the top surfaces of vials 602 and 604 are flush with one another, the swing arm could maintain UV sanitizer 800 at a constant height and would only be responsible for maneuvering UV sanitizer 800 in x and y directions. The close-up view of UV sanitizer 800 shows an internal structure of UV sanitizer 800 and how it includes a channel allowing needle 804 to extend through UV sanitizer 800 to engage and withdraw fluid from vial 602. UV sanitizer 800 works by emitting UV light in two opposing directions. UV sanitizer emits light upward to sanitize a surface of TSC 210 through which needle 804 extends and emits light downward to sanitize a surface of vial 602 through which needle 804 extends. This is important as handling of these cassettes prior to placement in cell processing device 100 can result in contaminates adhering to an exterior of the septa of various cassettes. Consequently, this sanitization procedure greatly reduces the likelihood of needle 804 being contaminated while withdrawing fluid from vial 602.

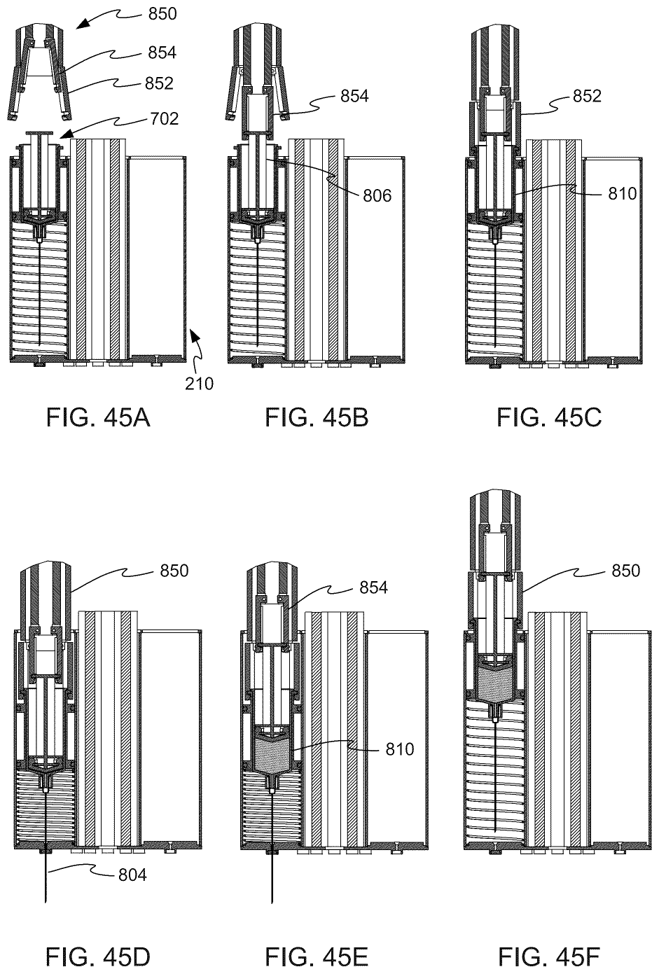

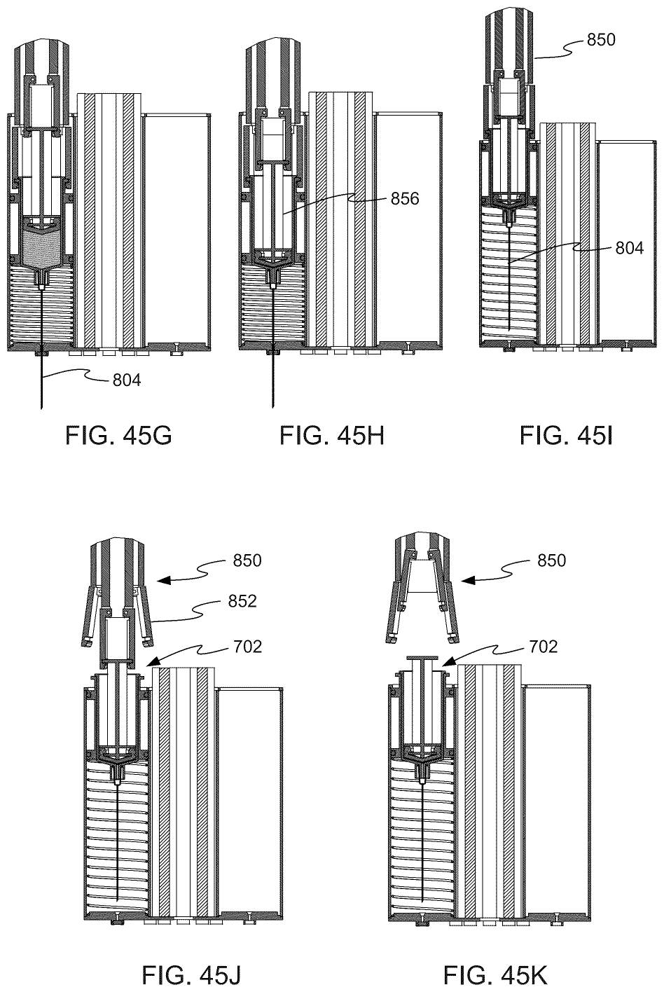

FIG. 44B depicts a configuration where after UV sanitizer 800 cleans the exterior of TSC 210 and top of vial 602, the needle extends through UV sanitizer 800, plunges into fluid contained within vial 602 and withdraws at least a portion of the fluid contained within vial 602. Syringe 702 may be pressed against spring 802 in order to engage vial 602 as depicted. Movement of syringe 702 downward may be accomplished by an actuator of cell processing device 100 positioned directly above TSC 210 that compresses syringe 702 against spring 802 by asserting a force on an upward facing surface of a plunger 806 of syringe 702. In some embodiments, TSC 210 can include a locking mechanism that prevents syringe 702 from travelling back upward once syringe 702 reaches a fluid withdrawal position within tube 808, as shown in FIG. 44B. Once syringe 704 is locked into the fluid withdrawal position that same actuator responsible for pushing syringe downward can assert an upward force on plunger 806 of syringe 702. The upward force may be applied to plunger 806 by the actuator using a suction cup, a vacuum head or a mechanical grip of the actuator until solution receptacle 810 is filled to a desired level. Once the desired level is reached the actuator may be withdrawn from TSC 210 and the locking mechanism holding syringe 702 may be released. In some embodiments, the locking mechanism can include a dampening element that prevents syringe from being abruptly slammed upward into a starting position and instead allows syringe 702 to be gently retracted into tube 808 of TSC 210. Alternatively, each spring may be tuned so that its force output only results in a gradual upward movement of a respective syringe. Operation of an alternative actuator configuration is described in the text accompanying FIGS. 45A-45K.

FIGS. 44C-44D show how sampling syringe 704 may be used to withdraw a sample from processing cassette 212 and transfer the retrieved sample into a sample vial 604 in RSC 202. FIGS. 44C-44D also demonstrate how UV sanitizer 800-1 is used when retrieving sample material from processing cassette 212 and how UV sanitizer 800-2 is used when depositing the retrieved sample material into sample vial 604. It should be noted that centrifuge well 114 may be equipped with its own swing arm for positioning UV sanitizer 800-1 between processing cassette 212 and TSC 210. While cell processing device 100 is described as including multiple UV sanitizers 800, in some embodiments cell processing device 100 includes only a single UV sanitizer 800 that is moved between centrifuge well 114 and vial cassette recess 126 by cassette positioning assembly 132.

FIGS. 45A-45K illustrate operation of an actuator 850 configured to manipulate reagent syringes 702 disposed within TSC 210. Actuator 850 may be positioned at an upper end of recess 134 of cassette positioning assembly 132 as shown in FIG. 3. FIG. 45A shows how actuator 850 includes a first gripping mechanism 852 and a second gripping mechanism 854. Second gripping mechanism 854 is carried within first gripping mechanism 852 and capable of moving independently relative to first gripping mechanism 852. First and second gripping mechanisms 852 and 854 each include fingers that are shown in an open state.

FIG. 45B shows how second gripping mechanism 854 of actuator 850 moves downward from its previous position in FIG. 45A and its fingers move into a closed state to secure second gripping mechanism 854 to a first lip at an upward end of a plunger 806 of reagent syringe 702. FIG. 45C shows first gripping mechanism 852 of actuator 850 having moved downward and fingers of first gripping mechanism 852 having moved to a closed state to grip a lip at an upward end of a solution receptacle 810 of reagent syringe 702.

FIG. 45D shows movement of actuator 850 downward to push a needle 804 of reagent syringe 702 out of a bottom end of TSC 210. While neither RSC 202 nor processing cassette 212 are shown receiving the needle of reagent syringe 702, these are omitted only to focus on the movement of actuator 850 and reagent syringe 702 relative to TSC 210. FIG. 45E shows movement of second gripping mechanism 854 upward while first gripping mechanism 852 remains stationary in order to move plunger 806 of reagent syringe 702 upward to draw fluid into solution receptacle 810 of reagent syringe 702.

FIG. 45F shows movement of actuator 850 upward to draw needle 804 and the solution reagent syringe 702 back into TSC 210 to allow for movement of TSC 210 by cassette positioning assembly 132. FIG. 45G shows how actuator 850 is configured to push reagent syringe 702 back downward to engage needle 804 into another cassette. Once needle 804 is engaged within the other cassette, FIG. 45H shows second gripping mechanism 854 having moved downward to press plunger 806 downward to expel fluids into the other cassette. FIG. 45I shows actuator 850 after moving upward to retract needle 804 into TSC 210. FIGS. 45J-45K show actuator 850 disengaging first and second gripping mechanisms 852 and 854 from reagent syringe 702. First and second gripping mechanism 852 and 854 can disengage from reagent syringe 702 either concurrently or sequentially. It should be noted that actuator 850, as depicted, is sized to work with reagent syringes but it should be appreciated that cell processing device 100 can also include a second similarly configured actuator scaled down in size for compatibility with sampling syringes 704.

The present invention includes a method for analyzing cell suspensions during the cell processing workflow to provide accurate cell counts, population distributions, and purity assessments. This analysis is achieved by integrating a Cell Analytics Module (CAM) within the cell processing system, capable of precise optical and biochemical cell characterization.

Analytical Sampling Process: The process begins with the TSC drawing a precise volume of cell suspension after the cells have been mixed to ensure uniformity. The precise volume of the sample (e.g., 5 μL, 10 μL, or a predetermined amount) is controlled programmatically by the system or chosen in advance by the operator. As described previously, the TSC is equipped with rotational capabilities, allowing it to align with various functional modules within the system, including the CAM. Once the TSC has drawn the sample, it rotates to align with the CAM, which may be integrated as a standalone analytical device or incorporated into one of the slots of the Reagent/Sample Cassette (RSC). The sample is then expelled through a microfluidic channel within the CAM, enabling precise analysis.

Cell Analytics Module (CAM): The CAM is a dedicated analytical unit designed to process cell suspension samples for real-time evaluation of cell characteristics, It comprises the following components:

Microfluidic Channel for Single-Cell Detection: The CAM includes a microfluidic channel with a diameter precisely engineered to allow single-cell passage. This ensures that cells flow individually through the channel, allowing high-resolution analysis. As each cell passes through the channel, an optical or impedance-based detection system measures key parameters, including cell diameter, which can be used to estimate the volume of individual cells. The CAM records the rate of cell passage, enabling the system to calculate the concentration of cells within the sample. By multiplying this concentration with the known sample volume, the total cell count in the suspension can be accurately determined.

Optical and Biochemical Detection of Surface Markers: The CAM integrates advanced optical systems and reagents for detecting specific cell surface markers. The system uses biochemical reagents capable of binding to target markers, such as CD3 (specific to T-cells) and CD14 (specific to monocytes). The detection system evaluates marker expression and provides a quantitative breakdown of cell populations. For instance: A T-cell is identified as being positive for CD3 and negative for CD14; A monocyte is identified as being positive for CD14 and negative for CD3.

Quantification of Cell Populations and Purity Analysis: Based on the detection of specific markers, the CAM quantifies the distinct populations of cells present in the sample. It calculates the number of T-cells and monocytes and determines the purity of T-cells by dividing their count by the total cell count in the sample.

Integration with System Control: The CAM is fully integrated with the system's central control unit. This integration allows real-time feedback of the analytical results, which can be used to optimize downstream processes, such as gene modification, washing, or formulation. The analytical data ensures that cell suspensions meet quality thresholds before proceeding to subsequent steps.

Portability and Design: The CAM is designed to fit seamlessly into the modular architecture of the cell processing system. It may be integrated within the RSC or included as a separate module accessible via the TSC. The modular design allows for easy replacement and maintenance while ensuring sterility and compliance with regulatory standards.

Advantages of the CAM Integration: The incorporation of the CAM into the cell processing system significantly enhances the accuracy and efficiency of cell characterization. The microfluidic design ensures single-cell resolution for concentration measurement, while the use of optical and biochemical detection methods enables precise differentiation of cell populations. By providing real-time analytical results, the CAM streamlines workflow and improves the reliability of therapeutic cell products, ensuring consistent and high-quality outcomes.

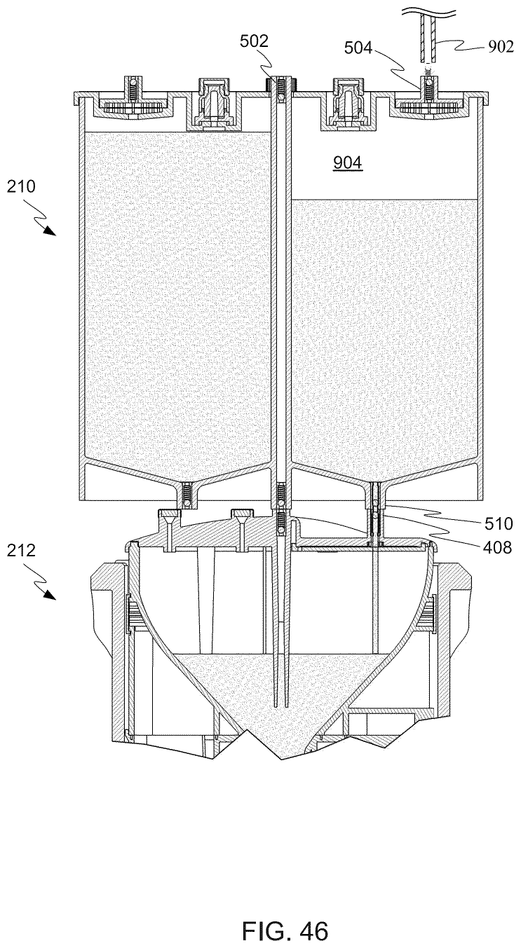

FIG. 46 shows a cross-sectional side view illustrating how PFC 204 can deposit solution into a main chamber 424 of processing cassette 212. In particular, it should be noted that UV sanitizer is not used in this step since processing cassette 212 has a high-quality filter capable of filtering out any contaminates that made their way on to fluid exit port 510 of PFC 204 and/or fluid port 408 of processing cassette 212. FIG. 46 also depicts a nozzle 902 attached to pneumatic port 504 that is responsible for supplying pressurized gas into a liquid chamber 904 of PFC 204 to cause solution disposed within liquid chamber 904 to exit out of fluid exit port 510 and enter processing cassette 212 through fluid port 408. In some embodiments, an amount of pressure supplied by nozzle 902 is tuned so that the filter disposed beneath fluid port 408 is able to keep up with a rate at which fluid is received.

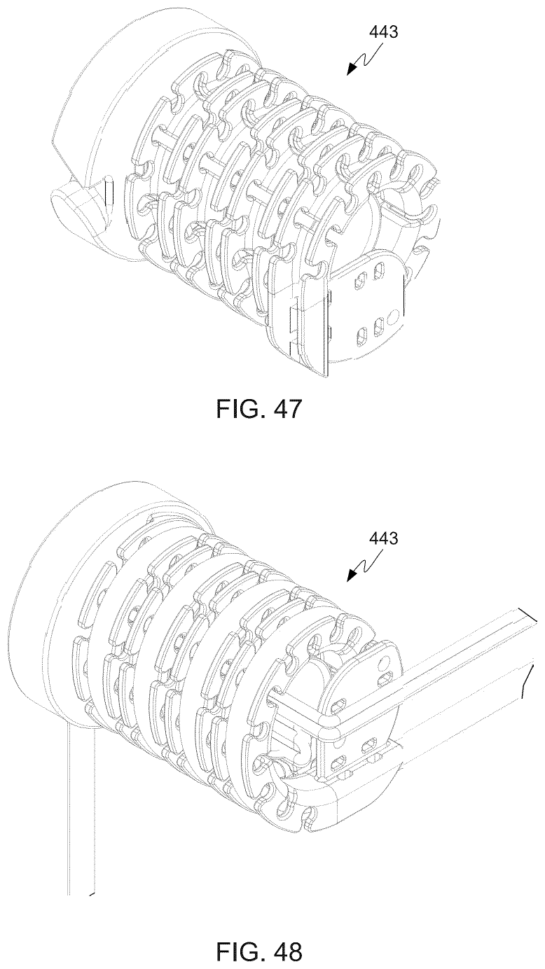

The system features a robust communication and power transfer design to ensure reliable operation within the centrifuge's dynamic environment, where noise and motion can create challenges. Suitable communication methods may include CAN-bus systems, robotic flex cables, or Ethernet connections. As illustrated in FIGS. 47 and 48, the system utilizes robotic flex and CAN-bus cabling 443 positioned near the centrifuge bucket's hinge, providing sufficient flexibility to allow the bucket to pivot up to 135° in each direction. The power cable supplies energy to critical components, such as the motor and heater, while also transmitting real-time data-such as the temperature of the aluminum components-back to the control system. This cabling is specifically designed to maintain effective data transmission even in electromagnetically noisy environments.

In one alternative embodiment, robotic flex and CAN-bus cabling (see FIG. 30) is strategically positioned near the hinge of the centrifuge bucket to facilitate movement. This setup enables the bucket to tilt in either direction, with a preferred pivot range of up to 135° but capable of extending to 165° or 180° in some configurations, and full 360° rotation in certain use cases. The power cable provides energy to the motor and heater while the CAN-bus delivers real-time feedback, such as the aluminum temperature, process sensor values, valve position and other operating parameters to the control system. The CAN-bus architecture ensures robust and noise-resistant communication, making it particularly suited for the dynamic and electromagnetically noisy environment of the centrifuge.

FIGS. 49A-49D illustrate an automated balancing system (auto balancer), utilized in the ACP. Turning to FIG. 49A and FIG. 49B, a cross-sectional view of a centrifuge wherein the bucket is in its mixing mode is shown. FIG. 49C is a cross-sectional view of a centrifuge when spinning, and FIG. 49D is a cross-sectional view of a centrifuge when stationary.

The automated balancing system enhances the centrifuge to remain in a balanced state as fluid levels change within the CPC 212. The automated balancing system achieves dynamic balancing by employing certain linear actuators, each with a moveable mass that allows adjustable balancing of variable-mass samples within the centrifuge, centrifuge bucket and CPC during operation. The use of additional actuators in alternative embodiments may provide fine-tuning capability, which is particularly effective at higher RPMs where torque requirements are greater. The CPC 212 includes a locking mechanism or tab to achieve stability and to ensure that the CPC is properly seated and remains stable throughout the process. Each actuator incorporates a locking mechanism to prevent unwanted movement of the automated balancing system when under load. This mechanism permits movement towards the center of rotation of the centrifuge but restricts it in the opposite direction when engaged thereby stabilizing the system during high-speed operation.

A 3-axis accelerometer, precisely aligned with the rotation axis of the centrifuge, measures vibrations and out-of-balance conditions in real time. If vibrations exceed programmed limits, the system automatically shuts off the electrical drive to the centrifuge motor and engages inductive braking to halt rotation. This accelerometer is part of a feedback loop that actively compensates for imbalances, with multiple accelerometers on different excitation axes providing enhanced signal accuracy. The accelerometer synchronizes with a rotation position encoder, enabling precise, rotation-referenced data collection at any speed. Fluid transfers into, out of, or within the system may occur when the centrifuge is stationary or when operating at various relative centrifugal forces, ensuring precision and minimizing turbulence. The balancing system employs a counterweight mechanism, where each full motor rotation corresponds to a 2 mm adjustment in the counterweight's position. By correlating accelerometer data with positional information from the encoder, the system achieves precise real-time indexing of vibration data with the motor's rotational position, allowing for dynamic balancing, improved stability, and enhanced motion tracking along the X, Y, and Z axes. Additionally, the system can transmit accelerometer data to any rotor, providing increased compatibility in certain embodiments.

One use case example of the automated balancing system is as follows: The process begins once the centrifuge bucket positions the CPC in an upright orientation. The PFC descends, docks with the CPC, and performs the required fluid transfer operations, including dispensing fresh fluids into the CPC, or receiving waste fluids from the CPC for transfer to the waste container. After fluid transfer is complete, the system determines the volume or weight of the cell suspension inside the CPC using integrated sensors. This measurement provides critical data to enable accurate balancing of the system. The auto balancer utilizes the data from the sensors to reposition the moveable mass along a threaded rod. The linear actuator adjusts the mass location precisely to minimize vibration during centrifugation. This balancing step ensures stable operation by counteracting any imbalance caused by variations in fluid volume or weight within the CPC. Only after the automatic balancing procedure is completed does the system allow centrifugation to resume. The precise balancing prevents excessive vibration, reduces mechanical stress, and ensures consistent sedimentation rates and fluid dynamics during centrifugation.

The balancing system is seamlessly integrated with the centrifuge bucket to enable precise rotational control and an active swing function for in-place mixing when the rotor is stationary. This system employs a motion-inducing mechanism that imparts reciprocal movement to the container along a predefined path, ensuring effective mixing of the contents. The motion actuator supports non-linear movement patterns designed to promote uniform mixing. Its velocity profile follows a sinusoidal trajectory, decelerating at the endpoints of the rocking motion to minimize turbulence and ensure gentle handling of sensitive materials. The actuator also features adjustable speed settings, enabling optimized mixing at specific intervals to achieve thorough homogenization. Furthermore, precise motor position control allows for rotational mixing, which can create a swirling effect to enhance mixing efficiency in certain applications. This versatile system supports a variety of mixing techniques, including rocking, tilting, and rotation, ensuring optimal content uniformity.

As shown in FIG. 16, in order to ensure precise temperature control and monitoring, temperature sensor 150 is integrated within the cartridge 212 and positioned near the liquid contents. The cell processing device 100 may be configured to add or subtract heat to keep solution 1022 preferably approximately at a temperature of about 37 C, however, in different embodiments temperatures other than approximately 37 C may be desired.

The cell processing device 100 has an improved temperature control for efficiently heating the biological materials without excessive energy waste. In some embodiments, bucket 120 may include a heating element capable of increasing a temperature of the material contained within processing cassette 212. In some embodiments this design incorporates a thin, anodized aluminum heating element to heat only specific areas, supported by low-conductivity materials like heat stabilized cast nylon and air gaps to prevent unnecessary heat loss.

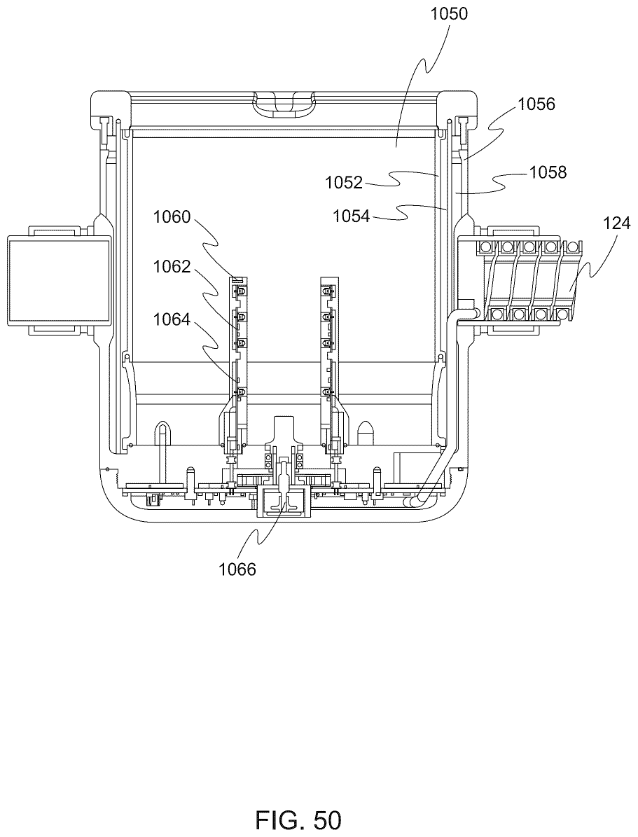

As shown in FIG. 50, the heating configuration features a primary heating element which is a thin, anodized aluminum cylinder 1050 approximately 2 millimeters thick, positioned close to the CPC 212 CPC where the biological materials are most concentrated, thus ensuring targeted heating without unnecessary energy expenditure. The aluminum is black anodized to improve its thermal emissivity which enhances heat transfer towards the cartridge contents. This heating element only covers a specific portion of the CPC 212, particularly the areas where the biological liquids are mostly concentrated, thus ensuring targeted heating without unnecessary energy expenditure. The heating element may be surrounded by a low conductivity insulative barrier made of heat-stabilized cast nylon or like materials and air gaps. The insulating properties of cast-nylon prevent heat from dispersing into the surrounding components, thus maintaining a focused heating zone. The insulative cast-nylon layer 1054 is about 2 millimeters thicks and circumferentially encapsulates the aluminum heating element to restrict heat flow outward. An aluminum tube with the heating element is wrapped by the insulative barrier. The heating element is adhered to the tube with pressure sensitive adhesive (PSA) and around that goes a mylar shrink belt. This mechanical belt is utilized to prevent delamination of the heater during centrifugation. In order to prevent heat dissipation, a black material is employed, and certain thermal path links are shut off utilizing plastic and an air gap. In this configuration, the cartridge design, incorporates a plurality of ribs which are not otherwise for structural purposes. The ribs facilitate heat conduction. This preferred configuration of the heating element retains the temperature inside even after the heat is turned off. The 2 mm thick, black-anodized aluminum warming cylinder 1050 and a film heating element 1052 maintain the temperature of cell solutions which are critical for cell viability and optimized processing conditions. A printed circuit board (PCB) 1060 affixed to the distal end of one of the sensor/emitter housings connects via spring pins on temperature sensor 150 of CPC 212 to monitor the temperature to ensure consistent thermal conditions. An additional air gap 1058 of approximately 6 mm is placed between the insulative layer 1054 and the outer bucket structure 1056. This air gap acts as a buffer to trap heat within the target region, further preventing thermal transfer to non-essential areas.

The purpose of the heating mechanism is to efficiently control the temperature of the biological material contained within the processing CPC while minimizing energy waste and preventing excessive heating of non-target areas. It is crucial for maintaining stable conditions at around 37° C., optimal for cell viability and processing, without causing thermal stress or denaturation of temperature-sensitive biological materials, and while minimizing heat dispersion to the exterior of the centrifuge bucket and the centrifuge itself. Further benefits are reduction of power consumption and the prolonging of component life.

As shown in FIG. 50, bucket 120 includes three optical sensor/emitter pairs 1062 to track cell sedimentation and to confirm correct valve rotation 1064. The bucket 120 is pivotally connected to the axis of rotation 124 for mixing cell suspensions in the CPC 212. The bucket further includes a valve rotation drive assembly 1066. Thus, the bucket 120 of the cell processing device 100 includes the black-anodized aluminum warming cylinder 1050 of 2 mm thickness for emissivity, the film heating element 1052 having approximately ½ mm thickness and air gaps.

In some alternative embodiments, cell processing device 100 can also include a refrigeration/heating unit capable of supplying chilled/heated air into centrifuge well 114. In some embodiments, the heat is conducted into the cassette via a conduction apparatus such as a thin black anodized aluminum tube around which is wrapped a heating element. To inhibit or minimize heat from transferring out to the remainder of the centrifuge bucket, a ceramic shell and air gap may be used. In some embodiments, the bucket is multilayered with materials of varying heat conductivity and insulation to specifically control the heat transfer.

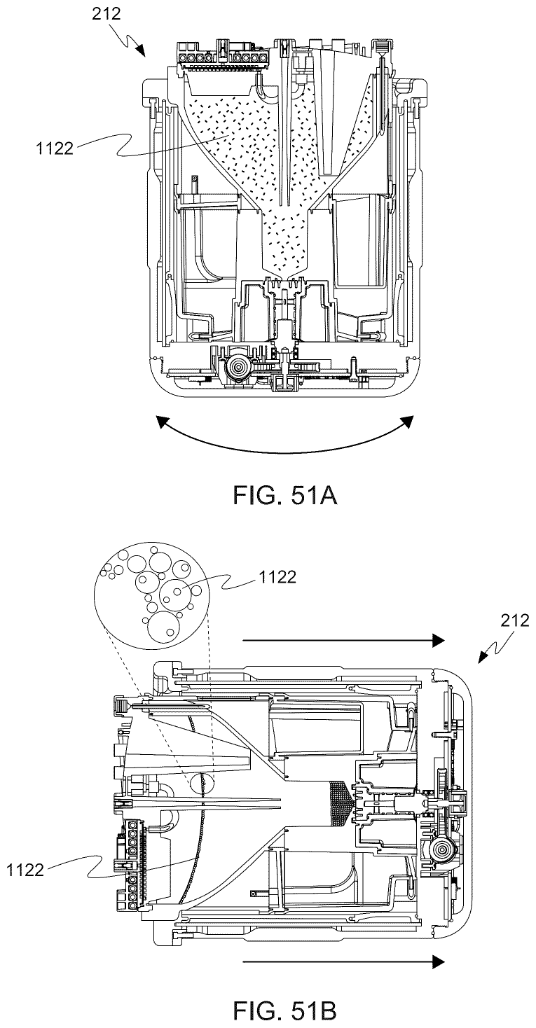

As shown in FIG. 51A, T-cells link to microbubbles 1122 during bucket mixing. FIG. 51B shows the bucket spun up to 250× g with the buoyant T-cells separate from sedimenting non-target cells. In the setup, the microbubbles facilitate a unique compression and release cycle for cell selection and concentration. The main steps include:

Centripetal Force during Spinning: As the centrifuge spins, centripetal force pushes non-target cells down towards the funnel's narrower end and ultimately to the sedimentation column 431. Here, the microbubbles maintain close proximity to each other and to the target cells thereby keeping the target cells from Sedimenting.

Buoyant Rebound and Separation: Cell buoyancy works against this downward force, causing cells to move upward in the funnel. As they rise, they encounter a widening cross-section of the funnel, which gives them more space to disperse. This separation step reduces the density of cell clusters and allows for a more even distribution of cells within the fluid.

This balance of forces—compression by microbeads against the funnel walls and buoyant separation as cells rise—allows the cells to be tightly packed and then gently separated thereby optimizing selection and distribution across the funnel. This design leverages both mechanical and physical properties to enhance precision in cell processing and isolation.

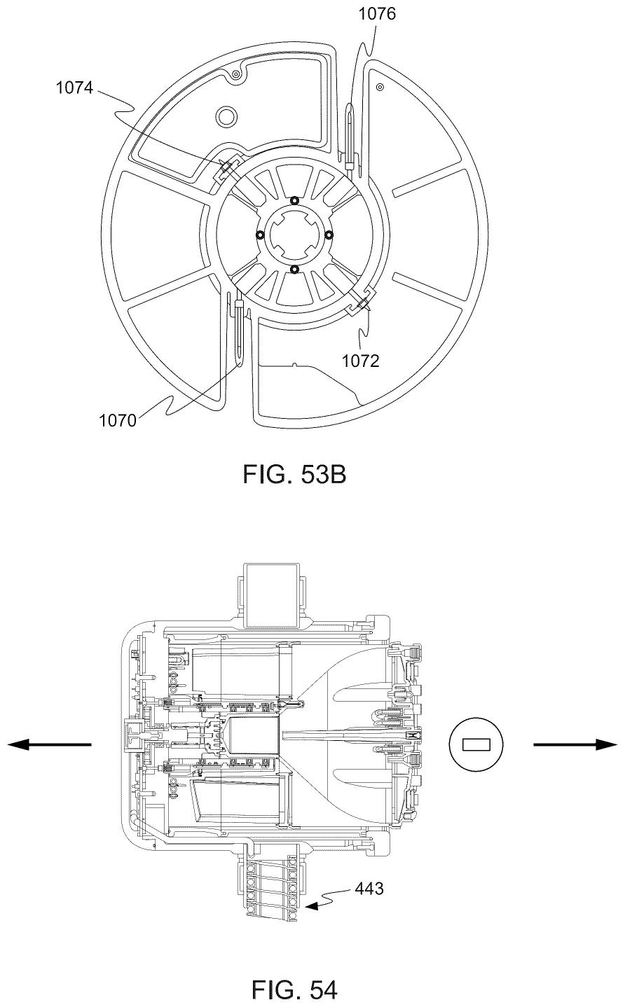

FIGS. 28 and 53A, show four primary rotational positions for CPC valves that correspond to different fluid transfer points within the cassette. The valve rotates to four positions such as reagent septa 1070, non-target cells 1072, 2^{nd} fraction T-cells 1074 and harvest septa 1076. Reagent Septa position allows reagents to enter the cassette, preferably for cell processing steps like washing, activation, or transduction. This position allows reagents to enter the bottom of sedimentation column of the cassette where the cells may, or may not, have been transferred by centrifugation of the CPC, likely for cell processing steps like washing, activation, or transduction. When in the Non-Target Cells location, the valve is positioned to separate and dispose of or transfer out non-target cells, ensuring that only the desired cell population remains in the cassette. Here, it sequesters non-target cells, ensuring that only the desired cell population remains in the main tapered or conical cell processing chamber of the CPC for subsequent processing steps. Harvest Septa position is used for harvesting the final cell product, allowing the selected and processed cells (e.g., CAR-T cells) to be extracted from the cassette. Second Fraction T-Cells position is designed for the isolation of a second fraction of T-cells, possibly for sequential processing or further refinement of the cell population.

As shown in FIG. 53B, In addition to these four primary positions, the diagram also indicates four other rotary positions, located in between the four locations listed above, each of which will prevent fluid transfer through the valve. These may act as “closed” positions where the valve rotation aligns with segments that block any fluid movement, ensuring containment and preventing unintended mixing or contamination.

The rotary valve positioning system can use up to three separate mechanisms to explicitly verify the valve is positioned correctly. These are a position sensor, an optical pass-through sensor, and an alignment sensor, as described below.

A position sensor on the valve drive shaft: The position sensor is used in conjunction with the servo motor controller to move the valve to a commanded angular position. Although in a preferred embodiment, any position sensor may be used, in one embodiment and optical encoder is used and reports the drive shaft position and the existing backlash/slop between the drive shaft and the valve rotor which may introduce uncertainty in the valve position. The encoder has an index position signal which defines 0° rotation which is calibrated during manufacturing using a fixture. For increased robustness, this is not the only position feedback of actual valve position used. In one embodiment the position sensor may have a 1:1 match with valve drive gear position.

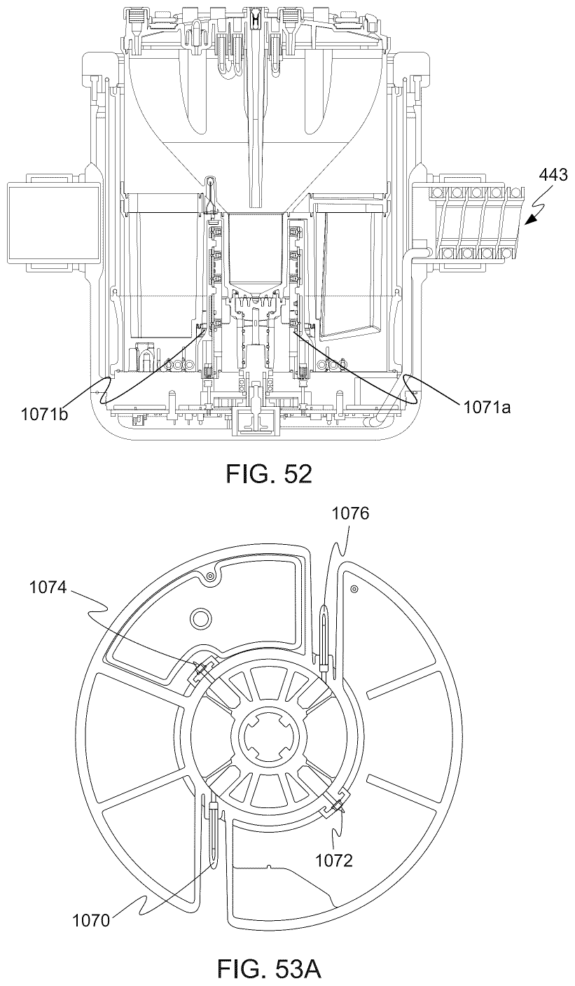

An optical pass-through sensor. As shown in FIG. 52, the alignment sensor is used for final valve position alignment, and includes a narrow slot in the valve rotor, which permits passage of an optical signal from an optical sender 1071a on one side to an optical detector 1071b on the other side (For the patent the position left-to-right of these sensors is likely not important, however, if it is important the numbers are reversed left-to-right in FIG. 52 from the actual position in the Trenchant bucket.). This emitter-detector pair provides line of sight confirmation through apertures in the valve stator, allowing the optical signal to pass through only when the valve is in the correct position of interest alignment between valve rotor and stator. In some instances, the correct alignment position is to open or close a port. In a preferred embodiment, the optical encoder directs valve rotation within +/−1.0° as depicted by FIG. 52. The alignment sensor signal depends solely on the relative positioning of the rotor and stator and is independent of backlash in drive components or variations in encoder or other position sensors. Furthermore, additional sensors may include but are not limited to Hall effect sensors, additional optical encoders, inductive sensors, magnetic or optical sensors.

The system employs zone sensors to indicate which of the four (or more) port locations is active during valve operation. While the implementation preferably includes four Hall effect sensors, the system is not limited to this sensor type; alternative position-sensing technologies, such as optical encoders, inductive sensors, magnetic sensor, or capacitive sensors may also be utilized depending on application requirements.

In the preferred configuration, the zone sensors interact with a single magnet located on the drive shaft gear to determine the general position of the valve relative to the ports. Each zone sensor corresponds to an active zone range, which is centered around an ideal port position. The active zone range may be between ±1° to ±20°. At any given time, only one zone sensor is active, signaling the valve's general alignment with one of the ports. When the valve is between ports, all zone sensors are inactive, ensuring the system recognizes that no port is currently engaged. This transitional state helps prevent misalignment or erroneous reporting. The zone sensors serve a critical role in valve positioning by confirming the valve is in the general location of the desired port. This confirmation enables the system to subsequently employ the optical alignment sensor for final, precise positioning. The optical sensor ensures the valve achieves the exact alignment needed, in some cases this alignment to open or close the selected port, leveraging its ability to detect alignment peaks with high accuracy.

By combining the general positioning capabilities of the zone sensors with the precise alignment detection of the optical pass-through sensor, the system achieves robust and reliable valve positioning. This redundant sensing approach allows for seamless operation even under conditions where minor mechanical backlash or tolerances might otherwise introduce uncertainty.

A simplified usage model for the rotary valve system is as follows. Starting position assumes the valve is in a “parked/closed”, no port open, position.

Initial Positioning to Nominal Port Location: A servo controller rotates the valve approximately 45° to the nominal angular position corresponding to Port A. This movement relies on pre-calibrated positional data. Next, the operation and accuracy of the encoder or other position sensors is verified by checking the zone sensors (e.g., Hall effect sensors or alternative technologies). At this stage, only the zone sensor for Port A is active, confirming the valve is in the correct general range. If no zone sensor is active, or more than one is active, the system will report a potential fault condition to the supervisory system controller for further diagnostics.

Precise Positioning Using Optical Alignment: The valve is rotated from the nominal position while monitoring the analog alignment sensor signal generated by the optical pass-through sensor. The system algorithm identifies the peak alignment signal (analog signal maximum), indicating precise alignment of the valve with Port A. Valve rotation is stopped at this peak position. Optionally, a final positioning algorithm may be executed to ensure maximum precision, accounting for mechanical backlash, tolerances, or drift.

Validation and Fault Detection: At this point, the system performs a positional cross-check to confirm alignment. Here, encoder position should indicate the correct angular position for Port A. A zone sensor confirms the general location. An optical alignment sensor validates precise alignment with the open port.

If all three detection mechanisms (encoder, zone sensor, and optical alignment sensor) agree, the valve is confirmed to be in the desired port-open position. If a mismatch is detected between any of these positional indicators, the system reports a fault condition to the supervisory system controller. The fault signal can trigger appropriate diagnostics, error handling, or corrective action.

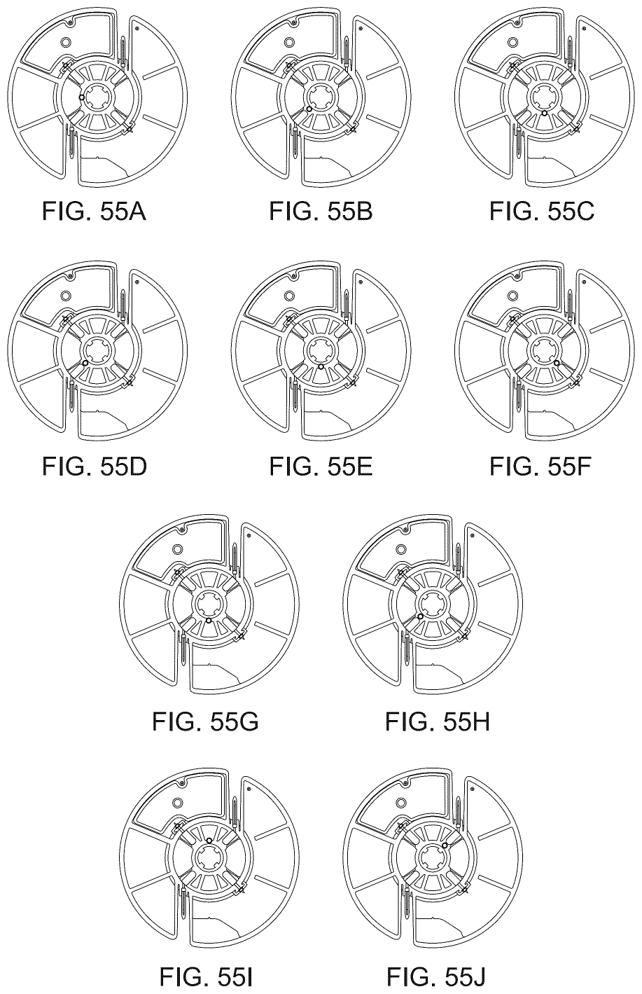

FIG. 54 shows the CPC 212 in the active mode. LED white light frequencies detect the white blood cells. FIGS. 55A-55J show the CPC valve rotating through a series of positions to facilitate the step-by-step processing of cells, reagents, and other components involved in CAR T-cell production. The valve rotation is mostly at 45° increments, with one 135° rotation in either direction. This allows for both fine and coarse adjustments to position the valve precisely for each task. Each position correlates with a specific action or step in the manufacturing workflow, allowing the device to isolate or combine materials as needed for optimal processing.

This rotation sequence demonstrates the CPC's versatility and automation, highlighting how the device can systematically carry out complex cell processing steps. By rotating the valve to control access to different compartments, the system enables a closed, automated, and highly efficient process for CAR T-cell manufacturing, where each step is fine-tuned to maintain cell quality, safety, and efficacy. There are four parked positions (1 3 5 and 7 are parked positioned) and there are four positions that allow cells to move.

Step 1: Filling, Buffer Addition, and Washing—Position 1, shown in FIG. 55A. The valve position allows for the introduction of buffer solution into the cassette, preparing the cells by washing them in preparation for further processing.

Step 2: Adding Linkers—Position 8, shown in FIG. 55B. This step enables the addition of linkers, which may be required for cell binding with microbubbles or other components.

Step 3: Mixing, Removing, and Adding Buffer—Position 1, shown in FIG. 55C. A combination step that allows mixing within the cassette, removal of spent or excess buffer, and addition of fresh buffer to maintain optimal conditions for cell health and activity. Buffer acts as a carrier to remove impurities and non-target components.

Step 4: Adding microbubbles (MB)—Position 8, shown in FIG. 55D. Microbubbles are added to assist in cell selection or separation. The Microbubbles may be manipulated to help concentrate, isolate or sequester specific cell populations.

Step 5: Mixing, Removing Buffer, Pressurizing, and Centrifuging—Position 1, shown in FIG. 55E. The valve position facilitates further mixing, buffer removal, pressurization, and centrifugation. This step likely aids in cell separation, concentration, or preparation for gene transfer.

Step 6: Sequestering Non-Target Cells—Position 2, shown in FIG. 55F. This step isolates and removes non-target cells, ensuring that only the desired cell population (e.g., T-cells) remains for subsequent processing.

Step 7: Adding Culture Media, Mixing, and Temperature Control—Position 1, shown in FIG. 55G. The valve introduces culture media to support cell growth, performs mixing, and regulates temperature (warming to 37° C. and cooling to 32° C.) to create favorable conditions for cell viability and activation.

Step 8: Adding Vector—Position 8, shown in FIG. 55H. A gene transfer vector, either viral or non-viral, is introduced to deliver the CAR gene into the T-cells, a critical step in the CAR T-cell therapy process, which enables the cells to target and eliminate cancer cells. The vector is introduced at the bottom of the cell suspension, allowing it to naturally rise through the small volume of T-cells. As both the vector and cells are in suspension, they begin to mix immediately upon contact, facilitating efficient gene transfer and uniform distribution throughout the cell population.

Step 9: Spinoculation, Washing, and Formulation—Position 3, shown in FIG. 55I. Spinoculation is used to enhance the gene transfer efficiency, followed by washing and formulating the cells for final harvest. Spinoculation helps bring the vector into close contact with cells, improving transduction rates.

Position 10: Harvest—Position 4, shown in FIG. 55J. The final step, where the CAR T-cells are collected and prepared for therapeutic use.

Key insights into the rotation system include controlled sequential processing, customizable angle and rotation control and closed-system integrity. In controlled sequential processing, the rotation positions enable a precise, sequential workflow thereby minimizing cross-contamination and optimizing the conditions for each process step. Further, in customizable angle and rotation control, the valve rotation is mostly at 45° increments, with one 135° rotation. This allows for both fine and coarse adjustments to position the valve precisely for each task. In Closed-System Integrity, the system design ensures sterility and containment. Also, this design is critical for aseptic cell processing, particularly when working with sensitive therapeutic cells.

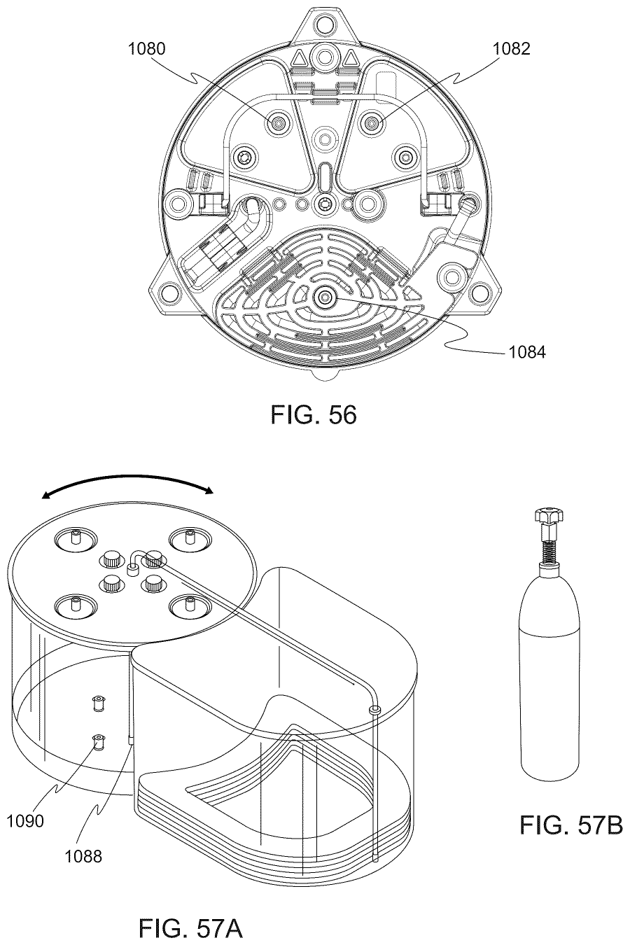

The ACP incorporates a rigorous pressure decay filter integrity testing (FIT) protocol to ensure the structural integrity and proper functionality of all filters within the CPC. As shown in FIGS. 56 and 57A-B, the FIT process is specifically used to validate aseptic transfer capabilities through filters, such as the 0.2 μm hydrophilic input filter located in the CPC lid. This testing is performed both before and after processing to confirm filter performance. Separately, waste removal and fresh media transfer are achieved via the PFC, which rotates, descends, and docks with the CPC lid. Fresh media enters the CPC through the input port, while waste exits through the waste outlet. The locations for FIT pressure decay tests are indicated as 1080, 1082, and 1084, while the pathways for waste removal and media input are shown as 1088 and 1090, respectively, in FIG. 57A. Additionally, FIG. 57B highlights a carbon dioxide-enriched container that facilitates the movement of fresh media into the CPC and waste media out at approximately 1.5 PSI. For filter integrity tests or degassing microbubbles, pressures of approximately 45.0 PSI are applied.

This process guarantees that fluid and gas exchanges with the CPC's interior occur exclusively through the filters, eliminating bypasses that could compromise sterility. The FIT procedure plays a pivotal role in maintaining an aseptic environment throughout the CAR T-cell manufacturing workflow, minimizing contamination risks and safeguarding the production of high-quality therapeutic products. Filter integrity is verified both prior to introducing patient cells and after completing the processing cycle, ensuring that no degradation or structural failures have compromised the sterility of the CPC

The automated pressure decay FIT process is independently conducted for hydrophobic and hydrophilic filters, which are critical components of the ACP's quality control system. Each filter is tested sequentially due to variations in surface area and decay rate thresholds, ensuring precise and individualized testing parameters. This protocol enhances sterility and reliability, ultimately improving the safety and efficacy of the CAR T-cell product.

Testing Process, Generally—The pressure decay test measures the pressure drop (AP) in an upstream volume connected to the filter over a predefined time interval. The process ensures confirms that the filter's flow rates are within permissible limits, ruling out obstruction or leakage. Hydrophobic and hydrophilic filters are tested separately. Due to differences in surface area, each filter requires unique pressure decay parameters and lookup values to determine acceptable decay times. The FIT protocol identifies potential issues, including low flow failures (indicating possible blockages or contamination in the filter or associated ports or passageways) and high flow failures (suggesting cracks, ruptures, seal failure, or leaks in the filter housing, CPC structure, or testing assembly). By testing each filter sequentially and employing precise decay thresholds tailored to the specific filter area, the ACP ensures comprehensive quality control. This robust testing protocol not only minimizes contamination risks but also reinforces the platform's ability to produce safe and effective CAR T-cell therapies.

Testing Process, Exemplary—(1) Pre-Process FIT: Before introducing patient cells, gas flows through sterile vents and the system is pressurized. (2) Decay Measurement: The pressure decay test calculates the drop in pressure over time using the formula: ΔP=DR×T×PaVupΔP=Vup DR×T×Pa, where: ΔP is the pressure drop, DR is the diffusion rate, T is the time, Pa is atmospheric pressure, and Vup is the upstream volume.

### Step-by-Step Testing Procedure

Close CPC Valve to prevent flow
Block Outlet Filter Port
Close Upstream Volume (UV) Valve
Close FIT Valve
Mate FIT Cassette to Hydrophobic Inlet Filter Port
Open UV Valve to Pressurize Upstream Volume to a starting pressure
Start Decay Timer & Open FIT Valve
Wait Decay Time and Record ΔP-decay Drop
Close PC FIT vent Valve
Close FIT Valve
PASS if ΔP-decay within min/max test limits or <Decay limit
Release all CPC pressure to complete test

Pass/Fail Determination: A Pass result is obtained if the pressure drops falls within an acceptable range. Failure results from either excessive or insufficient pressure decay, indicating potential filter issues.

Calculation for a representative test is shown below. For a starting upstream pressure of 45 psi, with these conditions, a 24.5 psi pressure drop would result in a final upstream pressure of 20.5 psi. A failing low flow filter would result in a higher final upstream pressure and a failing high flow filter would result in a lower final upstream pressure.

$$DR = 10 , 000 mL / minute ; Test Duration = 1 minute ; Vup = 100 mL$$

$$Δ P = ( 10 , 000 mL / minute * 1 minute * 14.7 psi ) / 100 mL = 24.5 psi$$

Post-Process FIT: After processing, a repeat FIT ensures that the filters remained intact throughout cell processing and did not suffer any performance degradation. The results of this post-process test are automatically logged in the batch record data, facilitating compliance with quality control and regulatory requirements.

In addition to running the filter integrity tests, the air pressure control system plays a critical role in managing and maintaining precise fluid flow rates within the system during key operations. It ensures controlled fluid dispensing into the CPC, removal of waste fluids, transfer of formulated gene-modified target cells, and specific manipulations during the selection and activation process, such as microbubble implosion. By regulating air pressure across multiple operations, the system ensures accurate and efficient fluid transfers and removals. The system operates by selecting air pressure in at least three distinct modes to optimize fluid handling and system integrity.

A first fixed pressure may be used for microbubble implosion and filter integrity Testing. A fixed air pressure is applied to collapse the microbubbles attached to target cells during the selection and activation stage. This removes the buoyancy imparted to the target cells, allowing them to settle efficiently for subsequent processing. The same fixed pressure (or a different pressure) may be used to test the structural integrity of both hydrophobic and hydrophilic filters on the CPC lid. This ensures the structural integrity of the filters that they are leak-free and able to maintain sterility throughout the process.

A second fixed pressure is associated with waste fluid removal. Positive air pressure is applied to propel waste fluids from the CPC through the central fluid waste tube and into an expandable, sealed waste container. This controlled pressure ensures efficient fluid removal without disturbing sequestered target cells at the bottom of the sedimentation chamber.

A third fixed pressure setting is utilized for fluid transfer from the PFC to the CPC. When the PFC is docked with the CPC, air pressure is applied to the fluid compartments within the PFC to control the transfer of fluids. This pressure ensures a regulated flow rate through the hydrophilic filter located on the CPC lid, enabling accurate delivery of fluids into the CPC for processes such as washing, reagent addition, or volume adjustments. It is further understood that these distinct fixed pressures could alternatively be supplied by a single electrically controlled regulator, which programmatically adjusts the pressure to meet the specific requirements of each process.

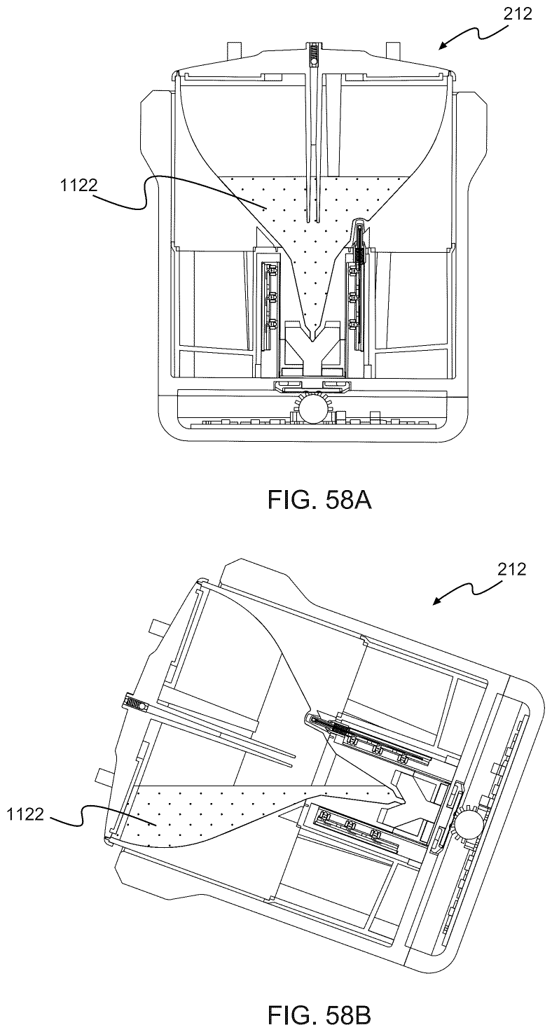

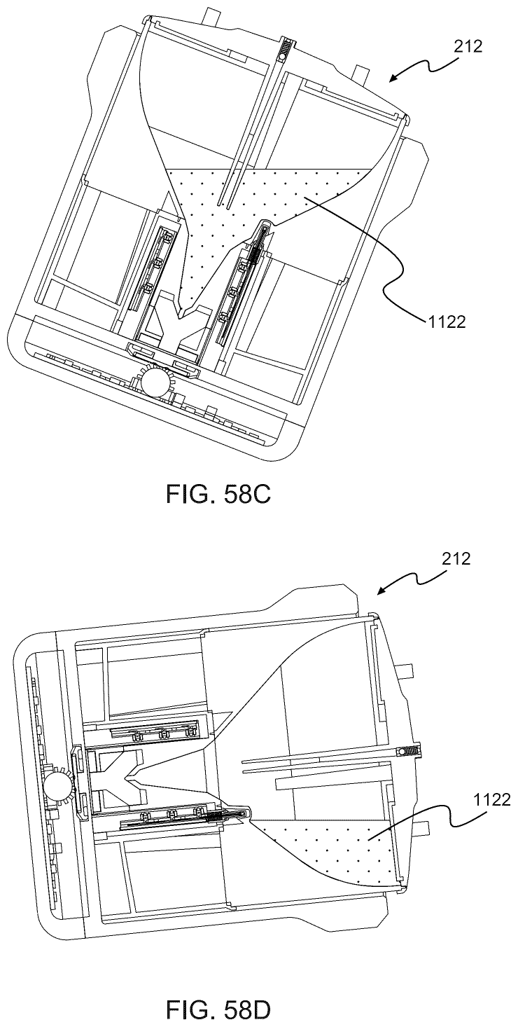

FIGS. 58A-58D show the cell suspension 1122 when the CPC 212 is being tilted. The As described above, the CPC may be tilted to facilitate mixing. The CPC is preferably reciprocally oscillated between 1° and 360°, and in some instances may be rotated an unlimited number of times. In some embodiments where ports or filters are found along the top lid of the CPC, the degree of tilt in either direction is inversely correlated to the level of fluid in the CPC. For instance, a 900 of tilt in either direction is typical for some a certain fluid level, but with reduced fluid, tilting beyond to for instance 135° may be possible, while in this embodiment a greater amount of fluid may only allow for some amount of tilting less than 90° in either direction.

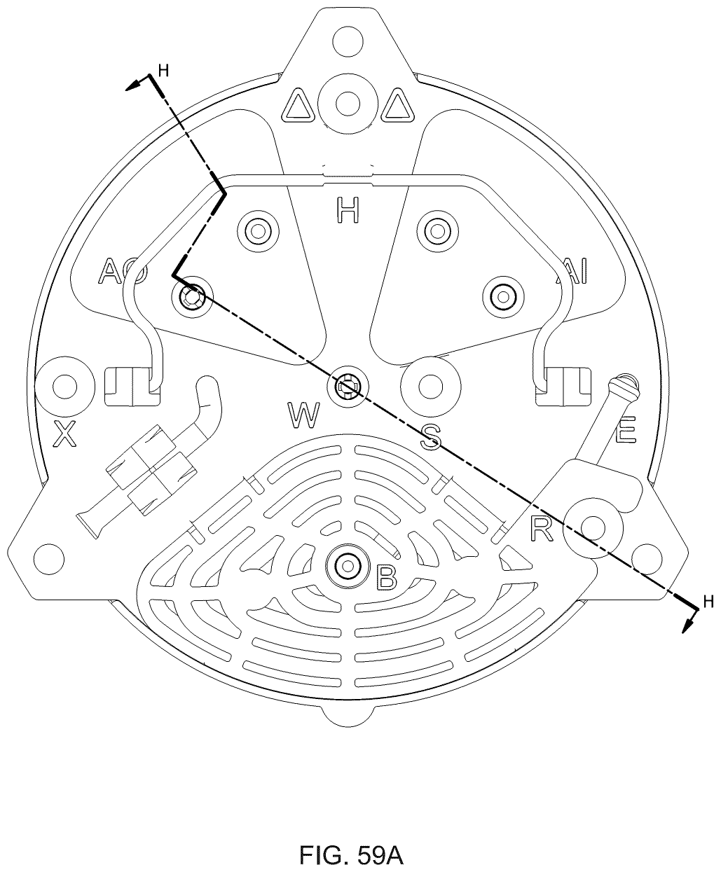

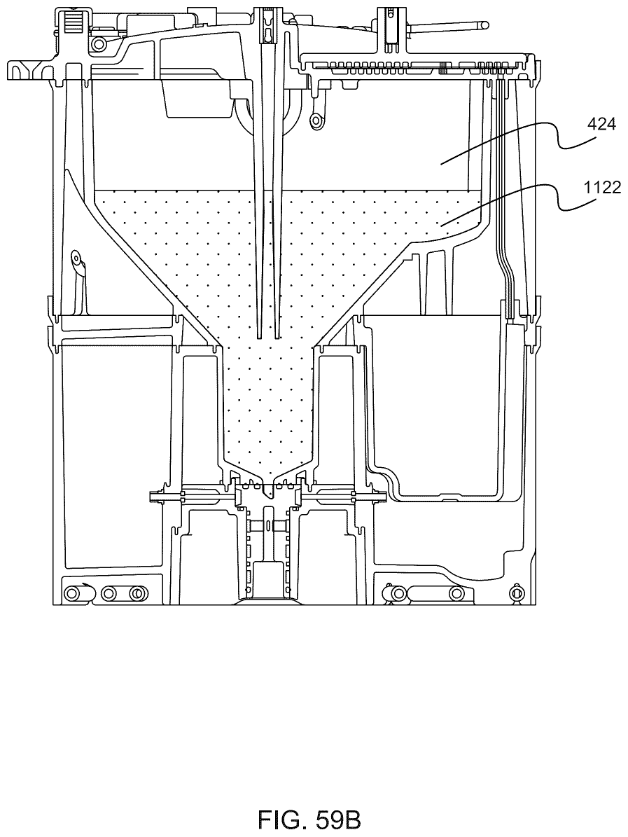

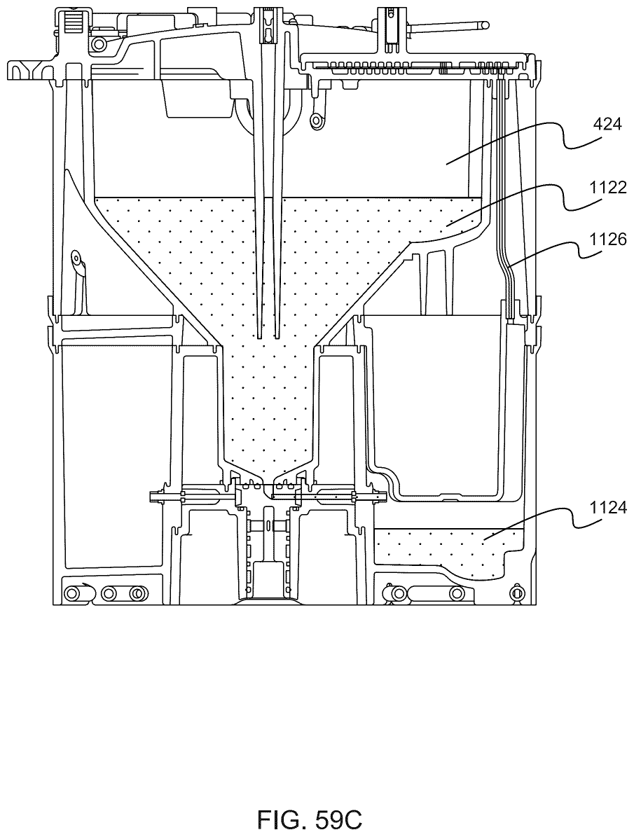

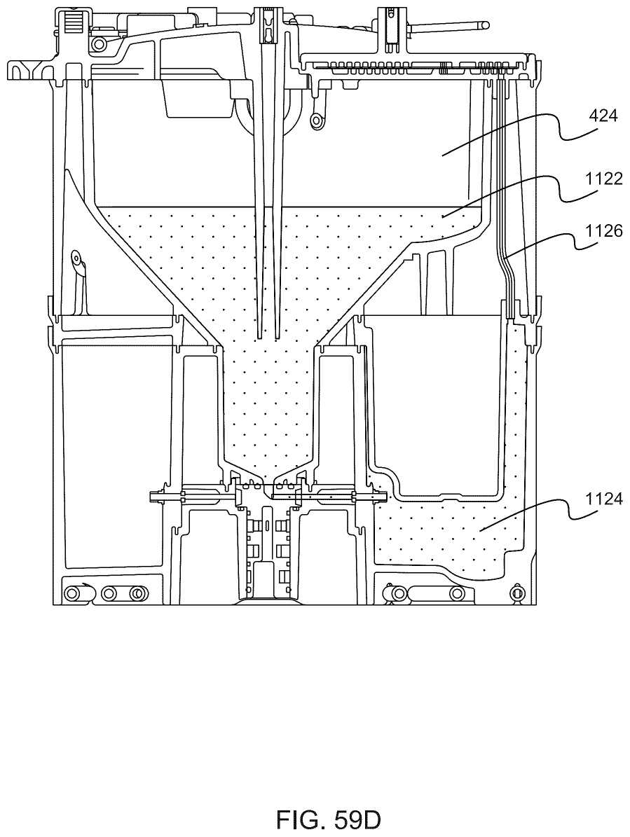

FIG. 59A a shows cutline H-H through the CPC 212 that is used for the cross-section images shown in FIGS. 59B-59D. In FIG. 59B, the cross section along H-H shows a cell suspension fluid 1122 in the main chamber 424. FIG. 59C shows some of the fluid 1122 moving into the 30 mL ancillary chamber 1124, extending up from which is a small standpipe 1126. In this embodiment the standpipe is 0.2 mL in volume. As the downward pressure from the weight of the fluid in the main chamber 424 fills the ancillary chamber 1124 under centrifugation, the pressure urges the fluid in the other chamber up the standpipe 1126. Thus, the small-bore standpipe design ensures precise fluid volume control under centrifugation, addressing challenges related to fluid displacement, sterility, and measurement accuracy.

In more detail, the second fraction compartment, capped with a lid, holds exactly 30 mL of fluid when full. During centrifugation, as fluid enters the compartment, the displaced air must exit. This is achieved through a thin bore tube that extends up the CPC's interior and connects to a port below a hydrophobic filter. The tubing's internal volume is less than 0.2 mL, ensuring precise air displacement while maintaining sterility by preventing communication with external air. When the CPC valve rotates from its park position to open the port leading to the 30 mL compartment, centrifugal force propels the cell suspension into the compartment. As fluid enters, displaced air travels up the thin bore tubing and is transferred into the main chamber 424. Any pressure differential that may be caused by this fluid movement can exit through the hydrophobic filter. The fluid rises within the thin bore tubing only to the same distance from the axis of rotation as the descending fluid level in the main conical or tapered compartment, ensuring equilibrium of centrifugal forces. Regardless of the centrifugation duration, this dynamic prevents fluid from overfilling or underfilling the compartment.

Key contributions of the standpipe are enhanced volume precision, measurement accuracy, and functional integration. The standpipe limits variability in fluid measurements. Any change in fluid height within the main chamber is mirrored exactly in the standpipe, ensuring precise volume control. The standpipe's small-bore volume (0.2 mL) ensures high measurement accuracy, with variability limited to ±0.1 mL. This precise design enables reliable determination of fluid levels without significantly affecting the overall capacity of the system. The design provides a simple, reliable, and highly accurate mechanism for determining the fluid volume in the secondary chamber during centrifugation. The standpipe system operates automatically, requiring no control mechanisms and is therefore cost-effective, minimally intrusive, and does not interfere with the overall functionality or capacity of the CPC.

This standpipe system is particularly beneficial for processes requiring high precision, such as cell separation and reagent handling, where minor variations in volume can significantly impact outcomes. Its integration within the CPC reinforces the closed-system design, minimizing fluid mismanagement and contamination risks. During operation, since radar or laser systems cannot monitor fluid levels while spinning, the process relies on empirically determined timing. By maintaining 50G centrifugal force and leaving the valve open to the compartment input for a standardized duration (e.g., 10+ seconds beyond the minimum), precise fluid transfer of the 30 mL volume is consistently achieved.

FIG. 59D shows the fluid from the main chamber 424 that continues to urge the fluid upwards in the standpipe 1126 until it equalizes with the fluid in the main chamber 424. The fact that this standpipe 1126 is so small (0.2 mL) means very little uncertainty as to the 30 mL volume of the other chamber, since such a small amount will need to move up the standpoint in order to equalize with the liquid level of the main chamber 424, regardless of the level.

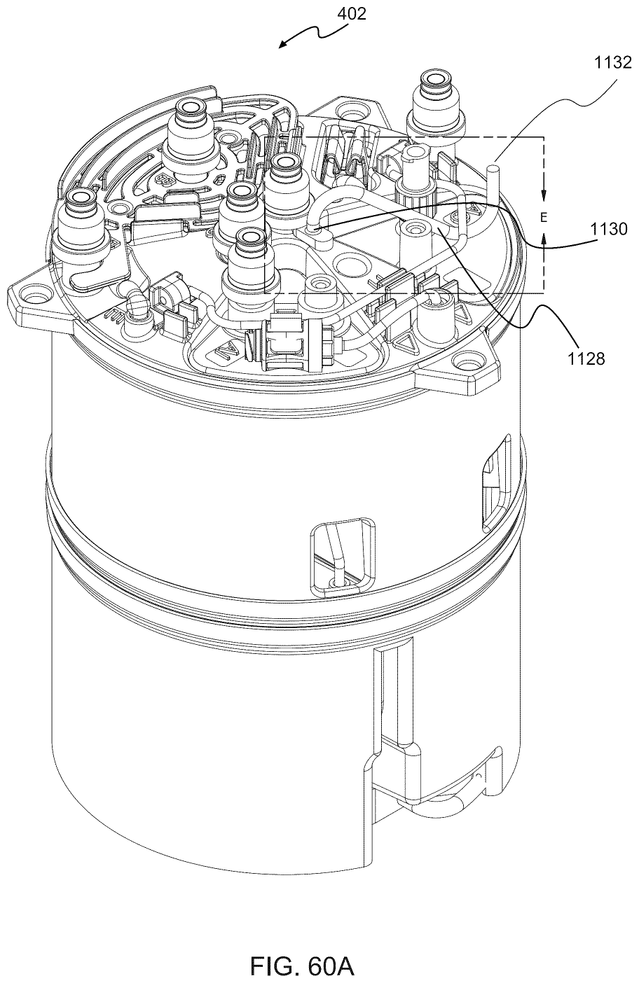

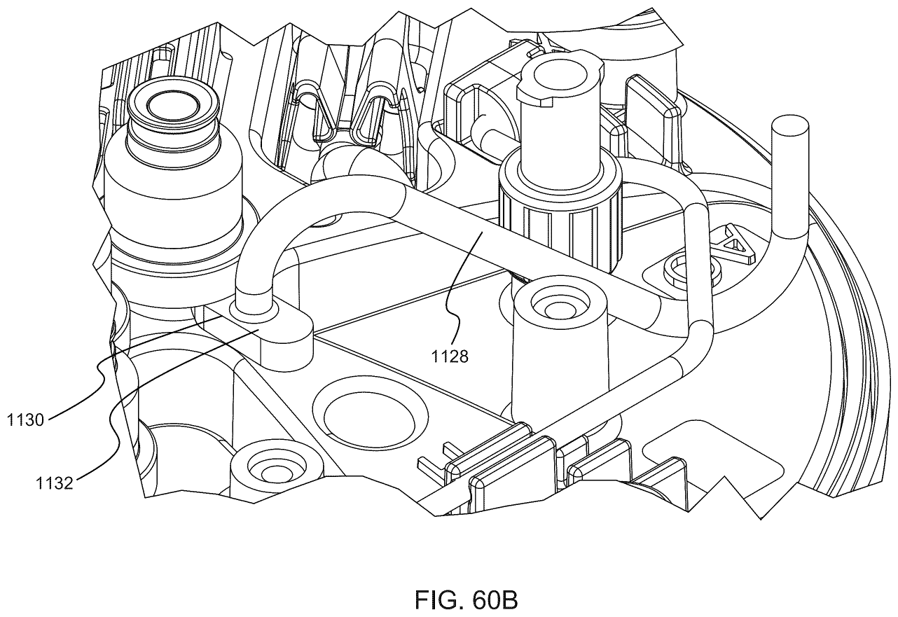

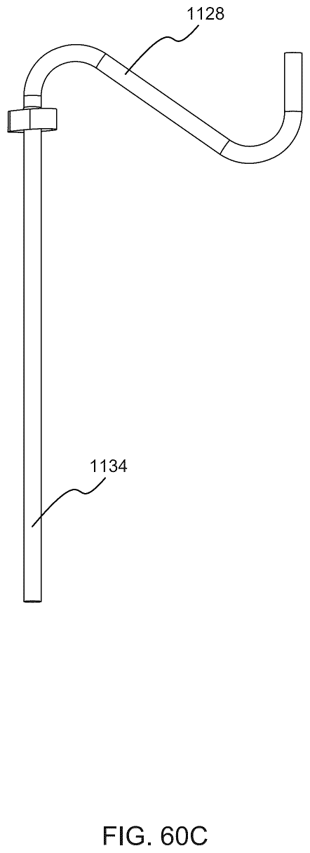

The dynamic nature of the CPC's weight, caused by fluid entering or exiting during operation, requires precise monitoring to ensure stability and accuracy within the centrifuge bucket. This is achieved through integrated weight sensors or fluid level detection systems, such as radar, laser, weight, or time of flight monitoring systems. In a preferred embodiment, radar technology is used to measure fluid levels with high accuracy. Here, a radar system is positioned above the CPC lid, where it emits radar waves through a guide rod. The radar waves reflect off the fluid surface at the precise point where the liquid contacts the rod. The radar system eliminates reflections from plastic walls, focusing only on the fluid surface for accurate readings. FIGS. 60A and 60B show this radar system 1130 connected to the cover 402. An S shaped channel 1128 directs radar 1130 from a radar emitter 1132, not shown.

The radar emitter/receiver 1130 (not shown) are configured to detect reflections from the fluid surface within a sensing range of 15 to 85 mm, wherein the detected fluid height is converted to a weight estimate for balancing purposes. The system software calculates the fluid level by subtracting reflection data between the emitter/sensor/rod interface and the fluid contact point along the straight portion of the rod that extends vertically through the CPC lid.

In one exemplary case, this utilizes a 61 GHz radar transceiver (RFbeam V-LD1) coupled with a polycarbonate rod that transmits radar waves. The radar measures fluid levels with exceptional accuracy, achieving better than 1 mm resolution across a range of 0 mm to 63.47 mm, corresponding to fluid volumes from approximately 15.4 mL to 350 mL. This embodiment ensures a resolution finer than 1 mm, with calibration required for low liquid levels to maintain this precision. Nonlinearities observed in the 0-3 mm measurement range due to end-of-rod reflections are corrected via software filtering algorithms. The impact of epoxy glue used to secure the rod to the lid is visible in the radar signal but is effectively filtered out through advanced signal processing. The radar's signal dispersion is controlled to ±10°, ensuring that only reflections from the fluid surface are captured, excluding interference from the containment walls. The rod's positioning is critical, avoiding contact with the containment except at designated fixation points to maintain measurement fidelity.

The radar system incorporated into the platform achieves a 99% energy reflection accuracy at the fluid contact point, ensuring precise fluid level detection. Real-time adjustments are facilitated during critical processing cycles, including fluid input, centrifugation, and extraction, as the radar detects changes in fluid height within key regions, such as the neck and funnel areas. This data allows the system to calculate fluid volumes with high precision, estimate weights accurately, and adjust operational parameters like rotational speeds and Relative Centrifugal Force (RCF) for optimal balance and performance. Measurement accuracy improves as fluid volume decreases, with a precision of ±4.5 grams at the maximum fluid level of 350 mL, improving to less than ±1 gram at 100 mL. Even at the highest fluid level, the measurement precision is well within the centrifuge's operational tolerance, which accommodates up to 50 grams of imbalance without compromising safety or performance.

FIG. 60C shows a rod 1134 for use with the radar 1130—the radar emitter/sensor (not shown) it as top right end of the rod 1128. The distance in the curved section up until the straight portion of the rod can be electronically removed. The straight length of the rod 1134 comes in through the cover 402 and just occupies a vertical distance. The reflection brings back 99% of the energy. The rod 1134 extends down into the liquid in main chamber 424, liquid does not move up the rod, however, the radar 1130 can detect the liquid level. This is a means for detecting the liquid level. Laser, ultrasonic detector, strain gauge are other alternatives.

In addition to load cells and/or radar/laser systems, a time-of-flight (ToF) monitoring system may be utilized to detect fluid levels within the CPC. A ToF system uses light pulses (e.g., infrared or laser) or ultrasonic waves to measure the time it takes for the signal to travel to the fluid surface and back to the detector. Based on this time, the system calculates the distance to the fluid level. This enables non-contact, high-precision detection of real-time fluid levels, ensuring accurate volume measurement and stable centrifuge performance.

In a preferred embodiment, the radar or other detection system engages only when fluid levels are changing or about to change. Alternatively, in some embodiments, the radar system activates only when the CPC is in its home position—vertically upright and motionless—ensuring consistent and interference-free measurements.

In use, the ACP automates the entire CAR-T cell manufacturing process, starting with leukapheresis and progressing through cell selection, activation, transduction, and final product purification. Key steps include washing, centrifugation, mixing, gene transduction, and multiple wash cycles to ensure a pure, viable, and potent cell product. Although there may be additional or fewer steps, the following is one examples of the platform process.

1. Apheresis Wash—Here, the initial leukapheresis sample is prepared by washing away unwanted materials while maximizing white blood cell purity and viability. Input is a leukapheresis sample containing red blood cells, white blood cells, platelets, and any other particulate matter. The leukapheresis sample is mixed with a buffer solution. The system removes unwanted components, such as excess antibodies, viral particles, and non-target debris, directing them to the waste container. Flow rates, temperature, and wash solutions are tightly controlled to preserve white blood cell viability and ensure a high-purity sample.

2. Selection and Activation—The objective in this step is to isolate target cells (e.g., hematopoietic stem cells, T-cells, NK cells) using biotinylated aptamer or antibody-based linkers and prepare them for activation and genetic modification. The system may use specific markers, such as CD3+, CD4, CD8, and CD28, to identify and select target cells. Alternative embodiments may use CD4 and CD8 markers alone, avoiding over-reliance on CD3. Biotinylated and non-biotinylated molecules play distinct roles in the cell processing machine's selection and activation system. Biotinylation is commonly used to functionalize molecules, such as aptamers or antibodies, with biotin groups that can bind strongly to streptavidin-coated surfaces or to streptavidin-linked microbubbles in the machine. This approach enables precise attachment of biotinylated molecules to microbubbles, enhancing target cell selection and activation by allowing these functionalized microbubbles to bind to specific cell surface antigens via biotin-streptavidin interactions. Conversely, non-biotinylated molecules are used when reversible binding is needed or when the application requires aptamers and antibodies that do not form permanent bonds with their targets. This combination allows the machine to balance stable binding during selection and activation steps with the flexibility to release or further process cells when needed, optimizing both specificity and operational flexibility in cell processing.

3. Linking Mechanism: Biotinylated linkers are introduced to bind to the target cell markers. Streptavidin-coated microbubbles are then added and attach to the linkers, forming a cell-microbubble complex that decreases the density of target cells, causing them to become buoyant in the fluid of the cell suspension.

4. Centrifugation: During centrifugation, the buoyant target cell-microbubble complexes migrate away from the axis of rotation (upward in the CPC), while non-target cells, being denser than the fluid of the cell suspension, sediment toward the bottom of the CPC's tapered compartment. The tapered or conical shape of the central chamber of the CPC increases in diameter as it is closer to the axis of rotation and ensures that the larger, buoyant target cell complexes avoid contact with descending non-target cells. Additional microbubbles may be added if necessary to improve cell buoyancy and separation efficiency. Target cells with attached microbubbles are concentrated at the top of the CPC, while non-target cells are sequestered in a sealable compartment at the bottom

5. Spinoculation and Transduction—In this embodiment, a CAR gene is introduced to target cells using viral vectors assisted by controlled centrifugation to optimize gene transfer efficiency. In detail, viral vectors are introduced to the CPC, targeting the selected and activated cells. The system employs spinoculation, where controlled centrifugation brings viral particles into close proximity with the target cells, enhancing transduction efficiency. The process may involve multiple steps, such as (1) centrifuging the target cells to improve vector penetration to the nucleus; (2) reciprocally pivot or rock the CPC back and forth to gently mix and redistribute cells and vectors, ensuring thorough interaction; and (3) re-centrifuge and repeat as necessary to improve gene transfer. The process is carefully timed and monitored to optimize transduction while minimizing cell stress. The outcome is gene-modified CAR-T cells generated with high transduction efficiency.

6. Wash and Harvest—The objective with this step is to purify and formulate a final cell suspension volume for the gene-modified CAR-T cells to meet therapeutic standards for purity, viability, and potency. The system performs multiple washing cycles to remove excess viral vectors, reagents, and particulate matter. Each wash significantly reduces contaminants. For example, introducing 150 mL of wash fluid, mixing thoroughly, and applying positive pressure transfers the contaminants to waste. In this case each wash achieves a 90% reduction in contaminants. Repeating the process three times achieves a 1000-fold reduction. After washing, the cells are concentrated to a convenient volume (e.g., 15 mL) and then adjusted with buffer up to the required formulation volume for harvest. Final quality control assessments confirm the purity, viability, and readiness of the CAR-T cell product. The outcome is a purified, concentrated, and ready-to-use CAR-T cell product suitable for therapeutic use.

The MBCSA (microbubble Cell Selection and Activation) technology enables sequential cell selection, addressing a critical limitation in current CAR-T production. Traditional methods, such as magnetic bead-based selection, are limited because once T cells are selected with magnetic beads, they cannot undergo further magnetic-based separations. In contrast, the MBCSA allows for de-gassing of the initial selection reagent, making it possible to sequentially select different cell sub-populations using secondary reagents. This capability facilitates a more precise and customizable approach to cell composition, allowing for enhanced control over T cell subsets in the final therapeutic product.

MBCSA Using Controlled Pressure—The ACP employs an innovative MBCSA technology that uses microbubbles to selectively bind and isolate target cells, such as T cells or stem cells, based on their surface markers. The microbubbles are in this instance small gas-filled bubbles with a lipid, polymer, or protein shell, often functionalized with specific molecules like aptamers or antibodies to bind to target cells. This process is facilitated by surface-modified, phospholipid-shell microbubbles that are stable, lyophilized, and easily reconstituted for use. By leveraging pressure to selectively collapse the microbubbles, the ACP enhances both the efficiency and effectiveness of cell selection, providing a high-purity, high-viability cell product that meets the rigorous standards required for therapeutic applications.

In the context of this application, lyophilized microbubbles refer to microbubbles that have been freeze-dried to preserve their structure and functionality for long-term storage and later use. Lyophilized microbubbles are more convenient to store, transport, and integrate into automated systems compared to their hydrated counterparts, which may require specific storage conditions. In their lyophilized form, the microbubbles are dehydrated under low temperature and vacuum conditions, removing moisture while retaining their structural integrity and biofunctional properties. Lyophilization stabilizes microbubbles, making them suitable for extended storage without loss of efficacy. Before use, the lyophilized microbubbles can be rehydrated (e.g., with a saline solution) to restore their functional form for binding to target cells within the cell processing platform. The functional groups on the microbubbles, such as biotinylated aptamers or antibodies, are preserved during the lyophilization process, ensuring specificity and binding efficiency. They then bind selectively and predictably to cell surface markers and facilitate separation, selection, or modification processes like spinoculation or controlled cell processing cycles.

Preparation and Targeting of Microbubbles—The microbubbles are coated with streptavidin, allowing them to link to biotinylated aptamers or antibodies that specifically bind to desired cell surface markers. This binding process causes the target cells to become buoyant, enabling easy separation based on their relative buoyancy within the CPC 212. As they rise, they encounter a widening cross-section of the funnel, which gives them more space to disperse. The balance of forces—compression of cells against the funnel walls and buoyant separation as cells rise—allows the cells to be tightly packed and then gently separated, optimizing selection and distribution across the funnel. This design leverages both mechanical and physical properties to enhance precision in cell processing and isolation This separation step reduces the density of cell clusters and allows for a more even distribution of cells within the fluid. To prepare the MBCSA reagent, lyophilized microbubbles are reconstituted with 6 cc of sterile saline and shaken for approximately 8 seconds to achieve a uniform suspension. Once reconstituted, the microbubbles retain stability within the fluid environment of the CPC 212, enabling efficient cell targeting and isolation.

Pressure-Induced Microbubble Collapse—A unique feature of the MBCSA process is the ability to disrupt the buoyancy of the microbubbles, releasing the bound cells by applying controlled air pressure. After the target cells have been isolated and bound to the microbubbles, the CPC's internal pressure is raised to an appropriate pressure, in one example approximately 2.5 atmospheres. This increase in pressure collapses the phospholipid shells of the microbubbles, effectively “popping” them and releasing the bound cells without causing cellular damage. The rapid collapse of microbubbles enables swift transition between cell selection and subsequent processing steps, streamlining the overall cell processing workflow The lipid shell of the microbubble is washed out later. The controlled pressure collapse is a critical aspect of the MBCSA technology, as it provides a gentle, non-destructive means of releasing the cells, allowing them to proceed to subsequent processing stages, such as washing, activation, or formulation of cells which remain viable and functional for therapeutic applications

### Aptamers

This system further integrates aptamers as high-affinity binding agents within the MBCSA system, targeting antigens such as CD3, CD8, CD28, and CD34. Compared to traditional antibody-based methods, aptamers offer cost-effective and rapidly customizable alternatives while demonstrating superior stability and reduced degradation under optimized conditions. For instance, aptamers can be selectively removed by introducing DNase to degrade the DNA and release the attached microbubbles, a flexibility not possible with antibodies. Additionally, antibodies can cause unintended physiological effects on cells due to internalization, a concern that does not arise with aptamer-based approaches. From an intellectual property perspective, aptamers offer a significant advantage as they can be synthesized in-house, avoiding reliance on commercially available antibodies.

Key findings highlight the system's effectiveness, with aptamer-linked microbubbles achieving higher purity and recovery rates than competing technologies. For example, the platform consistently produces a positive fraction of CD8+ cells with 95.1% purity and 89.1% recovery, outperforming traditional antibody and bead-based methods that typically yield approximately 50% recovery.

The ACP incorporates aptamers-short, single-stranded oligonucleotides (DNA or RNA)—that fold into specific three-dimensional shapes, allowing them to selectively bind to target molecules with high affinity. Compared to antibodies, aptamers are more versatile and cost-efficient to produce and modify, making them suitable for a wide range of targets, including proteins, cells, and small molecules. Their customizable nature supports tailored applications in biotechnology, diagnostics, and therapeutic processes.

The platform ensures aptamer functionality by employing precise folding protocols and optimized buffer compositions to maximize binding efficiency while minimizing degradation. This design achieves outcomes comparable to, or even surpassing, traditional antibody-based methods, providing a robust, scalable, and predictable solution for cell therapy manufacturing. The integration of aptamers guarantees high-quality cell products with minimal contamination, ensuring safe and effective therapeutic applications.

Aptamers are utilized alongside a microbubble system comprising lipid-shell, gas-core microbubbles, which confer buoyancy to target cells such as hematopoietic stem cells (HSCs), T-cells, and NK cells. During low-speed centrifugation, target cells bound to microbubbles float, effectively separating from non-target cells. This process is highly specific, with aptamers binding to unique surface antigens on target cells. The use of aptamer-linked microbubbles enhances precision in cell selection, enabling automated separation of target and non-target cells during centrifugation. By binding to specific cell surface markers, aptamers facilitate the isolation and concentration of desired cell populations, such as T-cells, within the CPC. Additionally, aptamers are engineered to work synergistically with microbubbles and other separation aids, further improving the efficiency and specificity of cell selection processes.

The aptamers integrated into the platform are structurally optimized to mimic the binding efficacy of antibodies, utilizing modifications such as dimerization and spacer enhancements. These adjustments maintain flexibility while ensuring high specificity for various cell markers. Moreover, aptamers exhibit reversible binding properties through tailored linkers, allowing the release of bound cells post-selection. This reversibility enables further purification steps or reuse of processed cells, particularly advantageous for T-cell-based therapies where sequential selection and activation of multiple subpopulations are required.

Integrating aptamers into the system delivers numerous benefits, including compatibility with automated, high throughput closed systems that ensure sterility, precision, and reproducibility. Aptamers' low immunogenicity compared to monoclonal antibodies (mAbs) enhances the platform's safety profile. In conjunction with microbubbles, aptamers enable the CPC to isolate and process cells efficiently while remaining adaptable for a wide range of therapeutic applications. This approach ensures the production of high-quality, safe cell therapy products tailored for clinical use.

The system contemplates use with the following advantages for aptamer designs:

Dual-stranded aptamers—The development of reversible aptamer linkers, such as dual-stranded aptamers, presents a promising avenue for enhanced flexibility in cell processing. These aptamers can be designed to detach by introducing a complementary strand with a higher binding affinity to the aptamer. This reversible mechanism would allow for (1) reuse of selected cells for additional processes or treatments, (2) Further purification steps to achieve higher specificity and purity in target cell populations, and (3) Expanded utility of aptamer-based cell processing across diverse applications, including research, manufacturing, and therapeutic contexts.

Thermally pre-treated aptamers—Thermally pre-treated aptamers undergo a controlled heating process to optimize their structural conformation, significantly enhancing their binding efficiency, stability, and application potential in cell processing workflows. By heating aptamers to a specific temperature (typically 70-95° C.) and rapidly cooling them, they fold into energetically stable and functional conformations. This prevents misfolding and aggregation, resulting in a homogenous population of active aptamers. Thermally treated aptamers exhibit stronger and more specific binding to target antigens, ensuring high-purity cell selection and reducing nonspecific interactions. Incorporating thermally pre-treated aptamers into the ACP's workflow would significantly enhance the precision and reliability of cell selection processes. Their adaptability and enhanced performance make them an ideal solution for applications demanding high specificity and efficiency.

Reversible Aptamer Design—Aptamers offer the potential for reversibility, further expanding their versatility. For example, a dual-stranded aptamer hybrid could act as a reversible linker. As another example, a single-stranded aptamer with an additional “tail” for attachment to a complementary strand could be used to facilitate detachment. By introducing a disrupting strand with higher affinity for the target antigen, the aptamer and attached microbubble could be effectively removed from the cell surface.

SELEX and Aptamer Development: The SELEX (Systematic Evolution of Ligands by Exponential Enrichment) process offers access to a vast library of aptamer candidates. Once funding is secured, a SELEX procedure will be conducted to identify aptamers specific to the desired antigens. This process will generate multiple aptamer candidates, enabling the selection of the best-performing sequences for use in cell processing. Thermally pre-treated aptamers can also be selected and optimized during the SELEX process to ensure they remain stable and functional under varying physiological and processing conditions.

The optimal range of conditions is as follows:

| Parameter | Optimal Conditions |
|---|---|
| Aptamer Design | Addition of Spacer-18 at 5′ end between biotin and bases |
| | to act as a hinge and enable easier access for MB |
| Aptamer Folding Buffer | 40 mM HEPES, 100 mM NaCl, 5 mM MgCl₂. |
| Aptamer Folding Temp. Profile | 10 min. 95° C., 15 min ice, 30 min 37° C., 45 min RT |
| Aptamer Folding Conc. | 10 nM |
| Aptamer Working Conc. | 0.5 nM |
| Aptamer:Cell Ratio | 0.075 nmol per 10 × 10⁶ T cells |
| Experimental Buffer | DPBS, 1% HSA. (in short, DPBS and protein) |
| | Addition of MgCl₂ to experimental buffer was |
| | tested to maximize aptamer stability but did not improve |
| | performance |
| | This is the main buffer used during the process |
| Aptamer Incubation Duration | 30 minutes |
| Aptamer Incubation Temp. | RT. 4° C. was tested to try and improve purity but had |
| | lower purity and recovery than RT |
| Aptamer Mixing Profile | Mix manually every 5 minutes |
| Microbubble:Cell Ratio | 450 μL per 10 × 10⁶ T cells |
| Microbubble Incubation Duration | 1 minute |
| Microbubble Mixing Profile | Manually Mix after addition and again at 30 seconds |
| Centrifugation Profile | 250 × g for 5 minutes |
| Positive Fraction Wash | Improves purity but reduction of recovery. Has not been |
| | used in recent studies since purity is now acceptable |
| | without it |

### Secondary Selection:

In one embodiment, an initial cell selection process is conducted to isolate T cells, followed by a subsequent selection step to refine a subpopulation, such as CD8+ or CD4+ T cells or memory T cells. This sequential selection can be performed using aptamers, where the first aptamer is digested and replaced with a second aptamer specific to the desired subpopulation. Alternatively, a primary selection reagent, such as microbubbles functionalized with specific antibodies or aptamers targeting T cells, is used to isolate the broader target population. A secondary selection reagent, comprising microbubbles coated with antibodies or aptamers for selective binding to CD8+ or CD4+ T cell subpopulations, is then employed to further refine the selection. Between selection steps, a degassing solution is utilized to remove the buoyancy of the microbubbles, ensuring efficient removal and preventing contamination of the primary cell population. This method facilitates precise, sequential isolation of target cell subsets for downstream applications.

### Use for Leukapheresis

This system's functionally closed design and advanced capabilities make it ideal for direct use in leukapheresis procedures, particularly by dramatically reducing target cell losses during manufacturing. Its precision enables efficient gene-modified CAR-T cell production, even with smaller white cell counts typically found in whole blood samples rather than leukapheresis collections. Leukapheresis involves the selective separation and collection of white blood cells granulocytes, lymphocytes, monocytes, and stem cells—from whole blood. The system's combination of closed-system sterility, controlled centrifugation, selective cell targeting, and automated quality control makes it highly suitable for leukapheresis applications. Its adaptability to handle smaller white blood cell counts and its ability to minimize target cell losses ensure efficient, high-yield recovery of leukocytes, supporting downstream processes such as CAR-T cell manufacturing and immunotherapy development, as detailed below:

Controlled Centrifugation and Sedimentation: The system optimizes the degree of target cell packing during spinoculation by dynamically adjusting centrifugation speeds. Cell detection sensors located in the sedimentation column monitor target cells as they sediment, ensuring precise control for improved packing and vector delivery efficiency.

Aseptic Transfer and Contamination Prevention: The closed-system design eliminates contamination risks by providing sterile fluid transfer pathways. Pressure decay testing ensures the integrity of both hydrophobic and hydrophilic filters on the CPC lid before cells are inserted. These features maintain cell viability and meet strict sterility standards required for leukapheresis and clinical applications.

Selective Cell Isolation and Enrichment: The system's aptamer and microbubble technology enable selective targeting and isolation of leukocytes. Aptamers or antibodies designed to recognize specific white blood cell markers (e.g., CD45 or CD34) can be applied to enrich leukocyte subpopulations. This targeted enrichment is particularly valuable in immunotherapy applications.

Cell Sedimentation Monitoring: The optical detection system, utilizing three optical sensors, provides real-time monitoring of target cell sedimentation. This ensures: Optimal centrifugal concentration of target cells, accurate packing of cells for enhanced vector exposure during gene modification processes, improved overall transduction efficiency.

Use with Senescent Cells

The described device can be adapted to target, isolate, and clear senescent cells, which accumulate with age and contribute to the progression of various age-related diseases. By specifically targeting these dysfunctional cells, the platform offers a novel approach to mitigating the adverse effects of cellular senescence on aging tissues.

The device may leverage aptamers specifically designed to bind to markers uniquely expressed by senescent cells. These markers include overexpressed surface proteins such as p16, p21, or SA-β-gal, which are characteristic of senescent cells. Using SELEX (Systematic Evolution of Ligands by Exponential Enrichment), custom aptamers can be tailored to selectively recognize these markers, ensuring precise targeting within a heterogeneous cell population.

Aptamers conjugated to buoyant microbubbles enable the selective binding and isolation of senescent cells. During low-speed centrifugation, microbubbles carrying bound senescent cells rise to the top of the funnel chamber. This buoyancy-based separation effectively isolates senescent cells from healthy, non-senescent cells in the sample, allowing for efficient enrichment and collection.

For enhanced specificity and purity, reversible aptamer designs may be used to allow multiple rounds of selection. After initial isolation, aptamers can be disrupted using complementary strands or higher-affinity reagents, releasing the bound senescent cells. This prepares the system for subsequent rounds of processing, refining the isolation of specific senescent cell subtypes for optimal purity and recovery.

The device facilitates the concentration of senescent cells within the funnel chamber, creating a high-density collection at the bottom. Once isolated, these senescent cells can be extracted or subjected to depletion strategies. Their selective removal has the potential to rejuvenate surrounding tissues by alleviating the inflammatory and detrimental effects associated with senescence.

The isolated senescent cells may serve as valuable resources for therapeutic research, enabling the development of cellular rejuvenation strategies. Alternatively, after clearing senescent cells from a patient's sample, the system could reintroduce purified healthy cells into the patient. This approach would provide a tailored cellular composition that promotes tissue repair and extends the patient's healthy lifespan, offering a transformative solution for combating age-related disease.

The ACP incorporates a comprehensive pre-operation diagnostic protocol to ensure the integrity and functionality of the CPC and associated components before any biological materials are introduced. When the clinician inserts the cartridge into the machine, the system automatically conducts a series of integrity and performance tests. If any failure is detected in the cartridge or its components, the machine prevents cell fluids from being introduced, prompting the clinician to remove and discard the defective cartridge. This ensures that only fully functional cartridges are used in the cell processing workflow. The initial testing sequence, which occurs before cells are loaded into the cassette, includes but is not limited to:

Filter Integrity Testing: Pressure decay tests are performed to confirm the structural integrity and functionality of all filters in the CPC.

UV Module Verification: The UV disinfection module is tested to ensure it radiates at the correct intensity for effective disinfection.

Centrifuge Diagnostics: The centrifuge system is assessed for operational readiness, including balance and motion parameters.

Mixing Actuator Testing: The rocking or mixing mechanism is tested for functionality and precise motion control.

Heater Calibration: The heating system is verified to ensure it can maintain the specified temperature profile during cell processing.

Upon successful completion of the diagnostic tests, a green light signals the operator that the system is ready for use. At this point, the operator may lift the lid, introduce the cells into the cartridge, secure the tubing, and snap the stub into place for processing.

Following the processing run, the machine conducts a secondary verification of critical sterility-related components, specifically re-testing the filters and the UV module. This post-operation testing ensures that the integrity of the CPC and the sterility of the process were maintained throughout the cell processing workflow. Other subsystems, such as the centrifuge, heater, and mixing mechanisms, are not re-tested post-run.

### Recovery and Purity

The disclosed system achieves exceptional recovery and purity rates in target cell selection, with a particular focus on CD8+ T-cell enrichment and isolation. Efficiency is calculated as the fraction of viable gene-modified cells retained from the initial cell population. For example, conventional manufacturing often begins with 2 billion T-cells, with up to 92.1% lost during processing over days or weeks. This leaves around 150 million activated T-cells, which are then expanded to 1 billion over 8 days. However, an additional 50% of these cells may be lost during reinfusion into the patient. The ACP minimizes these losses, compressing timelines and optimizing cell recovery for superior treatment outcomes

The platform disclosed herein demonstrates near 90% recovery and 95% purity for CD8+ T-cell selection, enabled by optimized aptamer functionality. This optimization involves controlled folding and binding conditions, including the use of buffer compositions such as 40 mM HEPES and 100 mM NaCl to effectively eliminate impurities and stabilize aptamer structures. Temperature protocols are also fine-tuned, incorporating a denaturation step at 95° C. followed by structured cooling to facilitate aptamer refolding into ideal conformations. Importantly, non-functional or misfolded aptamers are rendered inactive and do not disrupt the process.

The platform also excels in enrichment and depletion performance across various cell types. Mononuclear cell (MNC) recovery during enrichment achieves a rate of 95%, while CD34+ hematopoietic stem cells (HSCs) are recovered at an impressive 99%. Non-target cells, including red blood cells (RBCs), platelets (PLTs), and neutrophils (NEUs), are effectively depleted to levels of 99%, 84%, and 67%, respectively, ensuring a highly purified final cell population.

For T-cell isolation, the system produces CD3+ T-cell populations with a purity of at least 95% and recovery rates exceeding 95%. The resulting cell product contains 90-95% activated T cells to ensure immediate therapeutic efficacy and includes a substantial subset of memory T cells (20-30%) to enhance long-term in vivo durability. When HSCs are included in the workflow, they are enriched to similarly high purity and recovery levels, supporting their use in applications such as stem cell transplantation.

The present platform outperformed conventional platforms in both T-cell purity (99%) and recovery rates (90-95%), whereas alternative systems typically achieve only 50% recovery with approximately 90% purity. The emphasis on achieving exceptional purity is crucial, as contamination with non-T-cells or cancer cells in patient samples can compromise treatment efficacy. Notably, the entire process is completed within a single device at a single location, minimizing loss and contamination. The platform ensures that less than 1% of target cells are lost during processing, with further thresholds of less than 2% or less than 5% depending on operational parameters, demonstrating unparalleled precision and efficiency in cell therapy manufacturing.

### Comparative Efficiency Across Key Process Stages

Leukapheresis and Washing: The ACP achieves a 97.2% efficiency in leukapheresis and 95.3% in the washing stage, compared to industry standards of 50% and 49.9%, respectively. This significant improvement results from the platform's closed-loop automation and optimized fluid handling, which minimize cell loss and contamination risk.

Cell Selection and Activation: The Microbubble Cell Selection and Activation (MBCSA) process enables a 74.1% efficiency in cell selection and activation, compared to a standard efficiency of 39.4% in traditional magnetic bead-based or flow cytometry systems. The sequential selection capability of MBCSA ensures higher cell purity and viability, contributing to a refined therapeutic product.

Transduction: During the transduction stage, the ACP achieves a 94.7% efficiency, greatly surpassing the industry standard of 80%. The controlled mixing and even cell distribution provided by the CPC's geometry enable efficient vector exposure to cells, improving transduction rates while preserving cell viability. The mixing is to distribute the cells evenly, so the cells are better washed, avoiding sloshing, frothing and bubbles.

Harvest, Wash, and Formulation: The final stage of the process, including harvest, washing, and formulation, demonstrates a substantial efficiency improvement with the ACP, achieving a 65.0% efficiency compared to the industry standard of 7.9%. This improvement is due to precise fluid handling, reduced cell loss, and minimized handling steps, which collectively enhance the integrity of the final cell product.

Cumulative Process Efficiency: The cumulative process efficiency of the ACP across all stages of cell processing is 65.0%, compared to the current industry cumulative standard of 7.9%. This 823% increase in efficiency removes the need for extended ex vivo cell expansion, a costly and time-consuming step traditionally required to reach clinical doses in CAR-T cell production. By maintaining high viability and functionality of cells throughout processing, the ACP supports streamlined manufacturing with reduced cost and time requirements.

The ACP described herein provides several key functional advantages that enhance its utility in automated cell processing applications. These include:

Targeted Cell Selection: The use of aptamers enables selective binding and enrichment of target cells, significantly reducing contamination from non-target cells and improving purity for downstream applications. This precision ensures high-quality therapeutic outputs.

Microbubble-Assisted Separation: When combined with microbubbles, aptamers provide a buoyancy-based cell separation mechanism. This method isolates target cells via differential flotation during centrifugation, effectively segregating them from non-target cells with minimal loss.

Reversible Binding and Versatile Control: Aptamers can be engineered with reversible linkers or hybrid structures, allowing for the controlled release of bound cells following separation. This reversibility supports additional purification steps or reuse of cells, adding flexibility to the manufacturing process.

Compatibility with Automated Processing: The stability and customizable nature of aptamers make them highly compatible with automated, high-throughput cell processing platforms. This compatibility is essential for closed-system devices where sterility, precision, and repeatability are critical.

### Lot Release/Documentation

The platform may produce detailed lot release records for regulatory or other purposes. This capability supports personalized treatments, providing extensive data on each batch, similar to the extensive documentation requirements in large pharmaceutical manufacturing. The ACP continuously tracks and records a variety of critical parameters throughout the cell processing cycle, ensuring that each step adheres to the defined protocol and maintains optimal cell viability and product quality.

The tracked QC data may include, but is not limited to:

Identities: Protocol, patient, and cell source identifiers ensure each batch corresponds to the correct therapeutic requirements. Instrument and CPC identifiers verify the equipment and consumables used for each production cycle. Operator details recorded to maintain a full accountability record, allowing traceability and assessment of human involvement where necessary.

Cell Culture Parameters: Temperature, fluid composition such as dissolved oxygen ([O₂]) concentration, culture volume, and cell density are continuously monitored to maintain ideal cell culture conditions. Medium change times and volumes are recorded to verify compliance with specified growth conditions, ensuring cell viability and consistency across batches.

Physical Process Parameters: Centrifugation time-courses and applied forces are tracked to confirm that cells are adequately separated or concentrated according to protocol specifications. Mixing speeds, angles, frequencies, and durations are recorded to ensure uniform reagent distribution, efficient cell washing, and optimal cell suspension. Aspiration times and volumes are monitored to maintain sterile conditions and ensure precise fluid handling. QA sampling volumes and times for consistency, and for supporting reliable analytical testing across the manufacturing process. Pre- and post-processing filter integrity tests are conducted to verify that all filters maintain aseptic conditions, preventing contamination and ensuring product safety.

Reagent Information: Reagent identities, receipt and expiration dates, addition times, and volumes are documented to confirm the use of correct materials within their stability windows. Storage temperatures for all reagents are tracked to prevent degradation, ensuring that only viable materials are used in cell processing.

Automated Error Reporting and Release by Exception: The ACP incorporates an automated error reporting system that continuously monitors the data for any deviations from preset limits. If any parameter deviates from the established threshold, an error condition is immediately flagged, and the system records the details of the deviation. This automated detection allows for real-time error identification and corrective actions if necessary.

Release by Exception: The “release by exception” model implemented within the ACP is a transformative approach to batch release in cell therapy manufacturing. Traditionally, all batch records must be exhaustively reviewed before product release, a process that is highly labor-intensive and prone to human error. In contrast, the release by exception model only requires QC review for batches where deviations or anomalies are detected, allowing batches without errors to be automatically approved for release based on the system's verified compliance with all critical parameters.

The fully automated nature of the ACP reduces manual intervention, significantly lowering contamination risks and operational variability. Compared to traditional methods, the ACP accelerates the manufacturing process, enabling the production of a clinical dose of CAR T-cells in just 2.5 days without the need for time-intensive ex-vivo expansion. This streamlined approach ensures precise control over every stage of the process, optimizing flow rates, centrifugation forces, and timing to enhance product reliability, purity, and cell viability. Multiple washing cycles integrated into the workflow further reduce contaminants, ensuring a high-quality therapeutic product.

The compact and automated design of the ACP enhances accessibility, enabling deployment directly within FDA-licensed transplant centers. This co-location capability supports both research and clinical applications, allowing scientists to concentrate on gene construct development rather than labor-intensive manufacturing processes. Additionally, the ACP's robust quality control and aseptic processing steps ensure consistent and reproducible manufacturing outcomes. By simplifying and accelerating cell therapy production, the ACP has the potential to make life-saving treatments more accessible and affordable to a broader patient population.

The invention provides a multi-functional centrifuge bucket designed to hold a cell processing cassette (CPC) within an automated cell processing platform. The centrifuge bucket features a secure mounting interface that receives, aligns, and immobilizes the CPC during centrifugation and mixing, ensuring stable operation and proper alignment with fluid and gas input/output ports. Integrated into the bucket is a temperature control system capable of maintaining a controlled environment within the CPC, which includes heating elements and thermal insulation for consistent temperature regulation.

The bucket also incorporates a pivoting mechanism, allowing it to tilt or rotate along a defined axis. This motion supports efficient mixing and sedimentation of cell solutions within the CPC, minimizing shear forces on cells through smooth angular transitions and dynamic adjustments of angular velocity based on the properties of the cell suspension. Optical sensors within the bucket monitor sedimentation and cell distribution in real time, collecting data to enable automated adjustments and prevent unintended cell aggregation.

A data communication system is integrated into the centrifuge bucket, facilitating real-time communication with the CPC and associated sensors. This system supports the monitoring and control of centrifugation, mixing, and temperature parameters, ensuring consistent and precise cell processing conditions. The secure mounting interface further includes a quick-release locking mechanism to simplify the insertion and removal of the CPC. The centrifuge bucket's advanced features make it a versatile tool for cell processing, particularly in applications requiring high precision and reproducibility.

1. A multi-functional centrifuge bucket for holding a cell processing cassette (CPC) in an automated cell processing platform, comprising:
a. a secure mounting interface configured to receive, locate and immobilize the CPC within the centrifuge bucket, preventing displacement during centrifugation and mixing and ensuring alignment with fluid and gas input/output ports;
b. a temperature control system integrated into the bucket configured to maintain a controlled temperature within the CPC;
c. a pivoting mechanism connected to the centrifuge bucket, enabling the bucket to tilt or rotate along a defined axis;
d. optical sensors positioned within the bucket, configured to track suspended material sedimentation and monitor suspended material distribution in a fluid in real time, providing data for automated adjustments to control rates of suspended material sedimentation and prevent unintended suspended material aggregation; and
e. a data communication system integrated with the centrifuge bucket, enabling communication with the CPC and associated sensors, supporting real-time monitoring and adjustments to centrifugation, mixing and temperature parameters.

2. The centrifuge bucket of claim 1, wherein the secure mounting interface further comprises a quick-release locking mechanism to facilitate rapid insertion and removal of the CPC.

3. The centrifuge bucket of claim 1, wherein the temperature control system further comprises a heating element.

4. The centrifuge bucket of claim 1, wherein:
a. the suspended material are cells and the solution is a cell solution;
b. the pivoting mechanism incorporates a motion-inducing mechanism configured to impart a reciprocal motion to the container along a predetermined axis, enabling smooth and precise angular transitions that minimize shear forces on the cells in suspension during mixing and sedimentation; and
c. the motion-inducing mechanism adjusts angular velocity based on cell suspension properties and fluid dynamics, minimizing shear forces across varying cell types and densities.

5. The centrifuge bucket of claim 1, wherein the suspended material are cells and the solution is a cell solution, and wherein the optical sensors are equipped with multi-spectral imaging capabilities, configured to detect cell characteristics and to provide feedback to the automated control system for real-time adjustments to mixing, sedimentation, and temperature parameters based on detected cell characteristics.

6. The centrifuge bucket of claim 1, wherein the communication system is a CAN-bus cabling system that also delivers power.

7. A heating mechanism for a centrifuge bucket configured to hold a CPC in a cell processing platform, comprising:
a. a CPC containing a cell solution;
b. a temperature control module to modify or maintain a CPC temperature when the CPC is inserted into a centrifuge bucket;
c. an insulative containment layer surrounding the CPC, comprising:
i. a low thermal conductivity layer to inhibit heat dispersion to the exterior of centrifuge bucket; and
ii. an air gap positioned between the heating element and the outer structure of the centrifuge bucket, forming an additional thermal layer that retains heat within the CPC, while reducing thermal transfer to an external surface of the centrifuge bucket;
d. at least one thermal control sensor integrated within the CPC, positioned to be in close proximity to the liquid to monitor temperature and provide real-time data to a central control unit, wherein the control unit adjusts the heating element based on sensor feedback to maintain a temperature within a ±0.5° C. tolerance in the range of 20° C. to 37° C., ensuring stability under high-speed centrifugation conditions, while preventing overheating of a non-target area; and
e. wherein the heating mechanism and CPC construction transfers heat to the cell solution.

8. The heating mechanism for a centrifuge bucket according to claim 7 wherein the nontarget area is the exterior of the CPC.

9. The heating mechanism of claim 7, wherein the heating element is a black anodized aluminum further comprising embedded thermal insulation grooves designed to minimize thermal conductivity to the centrifuge bucket exterior while maximizing heat retention within the CPC.

10. The heating mechanism of claim 7, further comprising a fail-safe temperature regulation module integrated into the central control unit, configured to deactivate the heating element automatically if temperatures exceed a predefined threshold.

11. The heating mechanism of claim 7 wherein the low thermal conductivity layer is heat stabilized cast nylon.

12. A method of using a centrifuge bucket to process cells within a CPC in an automated cell processing platform, comprising the steps of:
a. securing the CPC within the centrifuge bucket using a mounting interface, ensuring the CPC is aligned and stabilized for precise fluid and gas exchanges during processing, wherein the CPC contains a cell suspension comprising cells, linkers, and microbubbles, and wherein a portion of said cells are target cells and wherein said cells have a distribution;
b. maintaining a controlled temperature within the CPC by activating a temperature control system integrated into the centrifuge bucket;
c. tilting and rotating the centrifuge bucket along a defined pivot axis to induce mixing of said cell solution within the CPC, evening said distribution of cells and attachment of said linkers or said microbubbles to said target cells;
d. applying centrifugal force to the CPC by initiating rotation of the centrifuge bucket promoting cell sedimentation and concentration of target cells at the base of the CPC; and
e. performing spinoculation at a specified rate and angle, thereby relocating target cells and vectors relative to each other and enhancing the uptake of genetic material into target cells through controlled vector exposure.

13. The method according to claim 12, further comprising monitoring said cell distribution within a sedimentation column of the CPC using optical sensors integrated within the centrifuge bucket, wherein the sensors track cell sedimentation rates and mixing uniformity and providing real-time data for process adjustments.

14. The method according to claim 12, further comprising communicating process data and adjustments to the automated cell processing platform via a communications link, enabling responsive adjustments to centrifugation parameters, temperature, and rotation angles to ensure consistent cell processing conditions.

15. The method according to claim 12, further comprising harvesting processed cells from the CPC upon completion of centrifugation and mixing steps, wherein the processed cells are collected through an output port on the CPC while the centrifuge bucket is stationary and vertically upright to facilitate post-harvest removal of the CPC.

16. The method of claim 12, wherein the tilting and rotating step includes moving the container along a reciprocating path and varying the speed at predetermined intervals to maximize homogenous distribution of said cell suspension and ensuring uniform exposure of cells to linkers or microbubbles.

17. The method of claim 12, further comprising the steps of:
a. applying pneumatic pressure within the CPC sufficient to implode the microbubbles and remove a buoyancy of the target cells;
b. sedimenting said target cells via centrifugation; and
c. sequestering said cells in the bottom of the sedimentation column to allow the removal of unbound linkers or microbubble fragments during the liquid waste removal step and preparing the target cells for subsequent processing.

18. The method of claim 12, wherein the rotation speed during centrifugation is adjusted in response to cell density measurements obtained from sensors integrated into the centrifuge bucket, preventing overpacking or aggregation of cells at the CPC base.

19. The method of claim 12, further comprising bringing the CPC back to the vertical and stationary position to allow the introduction of further reagents or buffers into the CPC through fluid transfer ports positioned in a lid of the CPC.

20. The method of claim 12, wherein the defined pivot axis for mixing cell suspensions includes a preset range of motion between 1 degrees and 180 degrees in either direction, with the range inversely proportional to a fluid level of cell solution within the CPC.
